(19) United States
(12) Patent Application Publication
(10) Pub. No.: US 2026/0234124 A1
(43) Pub. Date: Aug. 13, 2026

(54) Substituted Pyrazolyl-Pyridinyl Compounds as Ligand Directed Degraders of IRAK3

(71) Applicant: Celgene Corporation, Princeton, NJ (US)

(72) Inventors: Farid van der Mei, Cambridge, MA (US); Guobin Miao, Lexington, MA (US); Rulin Ma, Winchester, MA (US); Laura Akullian D'Agostino, Sudbury, MA (US); Kurt Armbrust, Cambridge, MA (US)

(73) Assignee: Celgene Corporation, Princeton, NJ (US)

(21) Appl. No.: 18/997,414

(22) PCT Filed: Jul. 24, 2023
(86) PCT No.: PCT/US2023/070825

§ 371 Date: Jan. 21, 2025

Related U.S. Application Data
(60) Provisional application No. 63/391,975, filed on Jul. 25, 2022.
(60) Provisional application No. 63/467,137, filed on May 17, 2023.

(51) Int. Cl. C07D 401/14; A61K 31/4545; A61K 39/39; A61K 47/54; A61K 47/55; C07D 417/14

(52) U.S. Cl. CPC C07D 401/14; A61K 31/4545; A61K 39/39; A61K 47/545; A61K 47/55; C07D 417/14

(57) ABSTRACT

Provided herein are compounds and compositions thereof for modulating IRAK3. In some embodiments, the compounds and compositions are provided for treatment of cancer.

### CROSS-REFERENCE TO RELATED APPLICATIONS

This application claims priority to U.S. Provisional Application No. 63/391,975, filed on Jul. 25, 2022, and U.S. Provisional Application No. 63/467,137, filed on May 17, 2023, the disclosure of each of which is incorporated herein by reference in its entirety for any purpose.

### FIELD

The present disclosure relates generally to compounds, compositions, and methods for their preparation and use of the compounds and compositions for treating cancer.

### BACKGROUND

The recruitment of immune cells to sites of injury involves the concerted interactions of a large number of soluble mediators. Several cytokines appear to play key roles in these processes, including interleukin-1 (IL-1). IL-1 produces proinflammatory responses and contributes to the tissue degeneration observed in chronic inflammatory conditions. IL-1 has also been implicated in the process of bone resorption and adipose tissue regulation. Thus, IL-1 plays a key role in a large number of pathological conditions including rheumatoid arthritis, inflammatory bowel disease, multiple sclerosis, diabetes, obesity, cancer, and sepsis.

IL-1 treatment of cells induces the formation of a complex consisting of the two IL-1 receptor chains, IL-1R1 and IL-1RAcP, and the resulting heterodimer recruits an adaptor molecule designated as MyD88, which binds to IL-1 receptor associated kinase (IRAK) (Wesche et al., J. Biol. Chem. 1999, 274, 19403-19410; O'Neill et al., J. Leukoc. Biol. 1998, 63, 650-657; Auron, Cytokine Growth Factor Rev. 1998, 9:221-237; and O'Neill, Biochem. Soc. Trans. 2000, 28, 557-563). Four members of the IRAK family have been identified: IRAK1, IRAK2, IRAK3, and IRAK4. These proteins are characterized by a typical N-terminal death domain that mediates interaction with MyD88-family adaptor proteins and a centrally located kinase domain. Of the four members in the mammalian IRAK family, IRAK2 and IRAK3 are thought to be catalytically inactive pseudokinases (Wesche et al., J. Biol. Chem. 1999, 274, 19403-19410), but the detailed roles of the two kinases are still largely unknown (Lagne et al., Structure 2021, 29, 238-251). Nonetheless, reports indicate the association of IRAK3 with negative regulation of TLR (toll-like receptor) signaling which is involved in detecting microorganisms and protecting multicellular organisms from infection (Kobayashi et al., Cell 2002, 110, 191-202). More recent studies have revealed the linkage between mutation or high expression levels of IRAK3 and various diseases such as asthma and cancer (Balaci et al., Am. J. Hum. Genet. 2007, 80 (6), 1103-1114; Kesselring, R. Cancer Cell 2016, 29 (5), 685-696), which suggests the potential of IRAK3 as a drug target and the need for IRAK3 binding small molecules.

Protein degradation is a highly regulated and essential process that maintains cellular homeostasis. Selective identification and removal of damaged, misfolded, or excess proteins is achieved through the ubiquitin-proteasome pathway (UPP). The UPP is central to the regulation of almost all cellular processes. Ubiquitination of the protein is accomplished by an E3 ubiquitin ligase that binds to a protein and adds ubiquitin molecules to the protein, thus marking the protein for proteasome degradation.

Harnessing the UPP for therapeutic use has received significant interest (Zhou et al., Mol. Cell 2000, 6, 751-756). One promising therapy uses proteolysis targeting chimeras, commonly referred to as PROTACs, to effect removal of unwanted proteins by protein degradation (Scheepstra et al., Comp. Struct. Biotech. J. 2019, 17, 160-176). PROTACS are ligand directed degraders that bring together an E3 ligase and a target protein that is to be degraded. These bivalent molecules usually consist of an E3 ligase ligand connected through a linker moiety to small molecule that binds to the target protein. A PROTAC positions the E3 ligase at the appropriate distance and orientation to the target protein, allowing the latter to be ubiquitinated. The ubiquitinated target protein is subsequently recognized by the proteasome, where it is degraded.

Accordingly, in one aspect, provided herein are compounds that target IRAK3 for degradation.

### SUMMARY

Descried herein, in certain embodiments, are compounds and compositions thereof for degrading IRAK3. In various embodiments, the compounds and compositions thereof may be used for treatment of cancer.

The present embodiments can be understood more fully by reference to the detailed description and examples, which are intended to exemplify non-limiting embodiments.

Embodiment A1. A compound of Formula (IA):

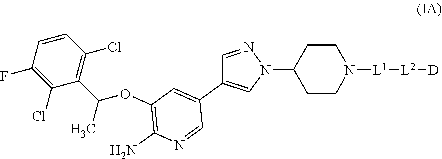

or a pharmaceutically acceptable salt thereof, wherein:
L¹ is —(CH₂CH₂O)ₙ— or a bond;
n is 1-10;
L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O;
D is

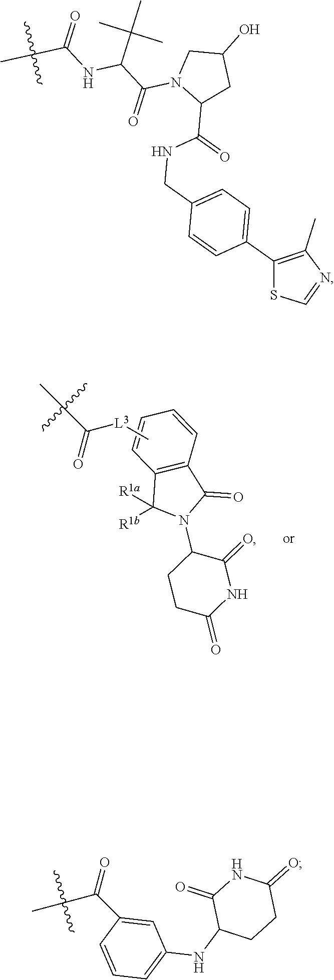

R^{1a} and R^{1b} are each H or are taken together to form an oxo; and
L³ is —CH₂O— or a bond.

Embodiment A2. The compound of embodiment A1, or a pharmaceutically acceptable salt thereof, wherein the compound is of Formula (I):

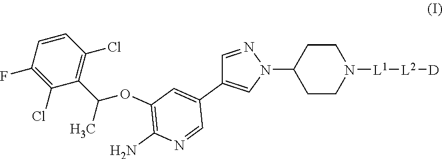

wherein:
L¹ is —(CH₂CH₂O)ₙ— or a bond;
n is 1-10;
L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O;
D is

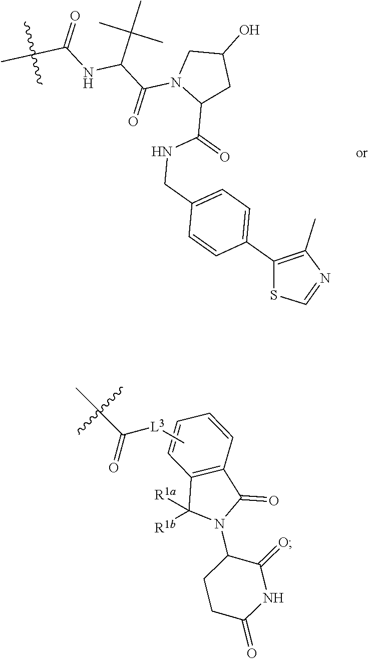

R^{1a} and R^{1b} are each H or are taken together to form an oxo; and
L³ is —CH₂O— or a bond.

Embodiment A3. The compound of embodiment A1 or A2, or a pharmaceutically acceptable salt thereof, wherein:
L¹ is —(CH₂CH₂O)ₙ—; and
n is 1-7.

Embodiment A4. The compound of embodiment A1 or A2, or a pharmaceutically acceptable salt thereof, wherein:
L¹ is a bond.

Embodiment A5. The compound of any one of embodiments A1-A4, or a pharmaceutically acceptable salt thereof, wherein:
L² is C₁-C₃ alkylene, —(C₁-C₃ alkylene)N(H)—, 10- to 12-membered spiro heterocyclylene, or a bond, wherein the heterocyclylene contains 1-2 nitrogen atoms.

Embodiment A6. The compound of any one of embodiments A1-A5, or a pharmaceutically acceptable salt thereof, wherein:
-L¹-L²- is

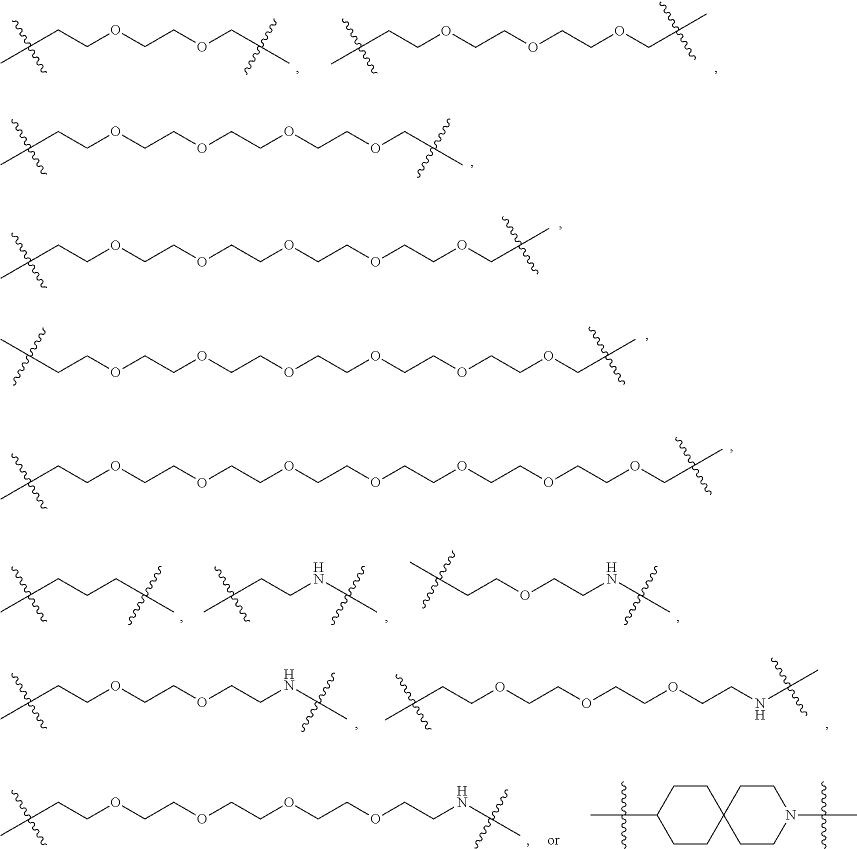

Embodiment A7. The compound of any one of embodiments A1-A6, or a pharmaceutically acceptable salt thereof, wherein:
D is

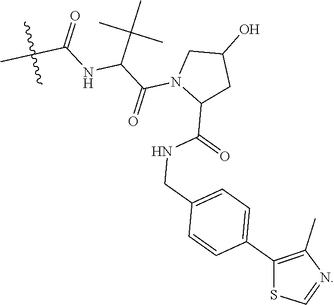

Embodiment A8. The compound of any one of embodiments A1-A6, or a pharmaceutically acceptable salt thereof, wherein:
D is

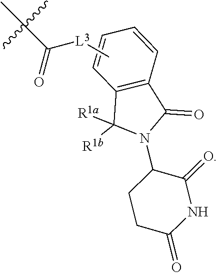

Embodiment A9. The compound of embodiment A8, or a pharmaceutically acceptable salt thereof, wherein:
R^{1a} and R^{1b} are each H.

Embodiment A10. The compound of embodiment A8, or a pharmaceutically acceptable salt thereof, wherein:
R^{1a} and R^{1b} are taken together to form an oxo.

Embodiment A11. The compound of any one of embodiments A8-A10, or a pharmaceutically acceptable salt thereof, wherein:
L³ is —CH₂O—.

Embodiment A12. The compound of any one of embodiments A8-A10, or a pharmaceutically acceptable salt thereof, wherein:
L³ is a bond.

Embodiment A13. The compound of any one of embodiments A8-A12, or a pharmaceutically acceptable salt thereof, wherein:

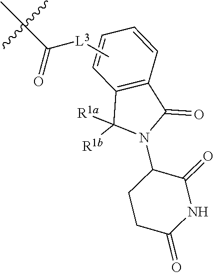

is

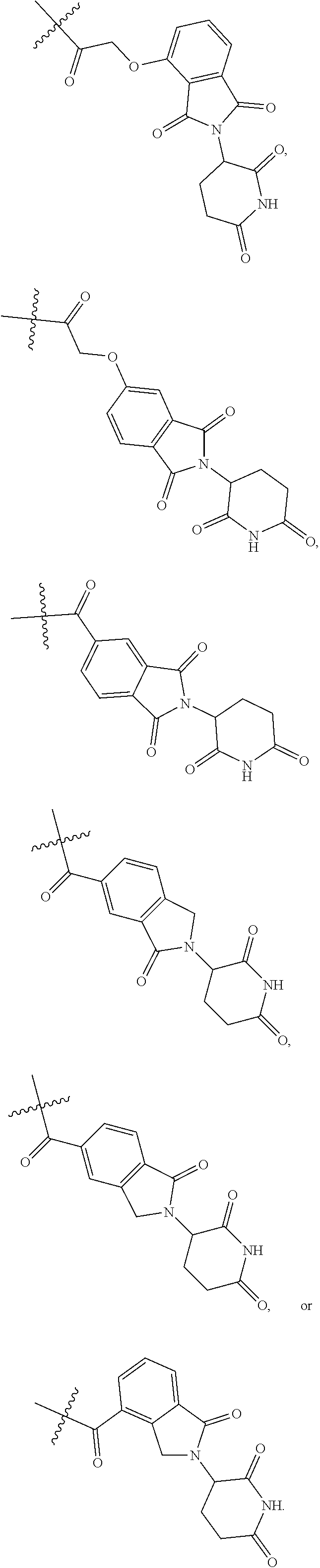

Embodiment A14. The compound of any one of embodiments A1-A6, or a pharmaceutically acceptable salt thereof, wherein:
D is

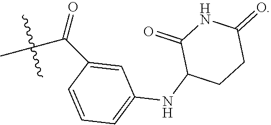

Embodiment A15. The compound of any one of embodiments A1-A14, or a pharmaceutically acceptable salt thereof, wherein the compound is of Formula (II), (III), or (VII):

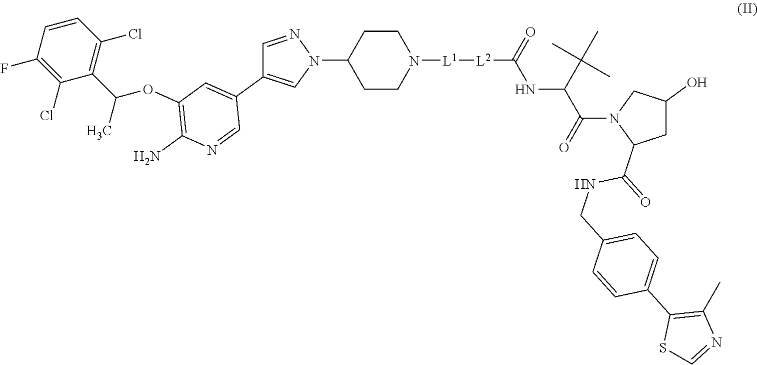

wherein:
L¹ is —(CH₂CH₂O)ₙ— or a bond;
n is 1-10; and
L² is C₁-C₆ alkylene;

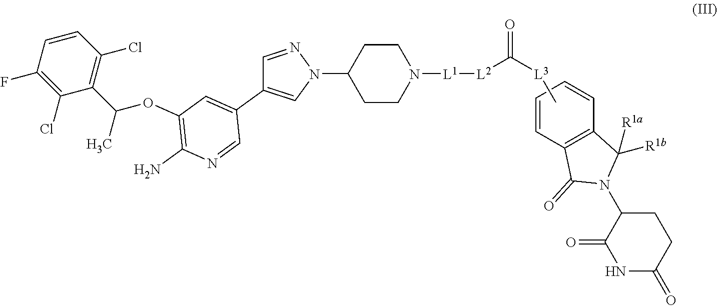

wherein:
L¹ —(CH₂CH₂O)ₙ— or a bond;
n is 1-10; and
L² is —(C₁-C₆ alkylene)-N(H)—, 6- to 12-membered Spiro heterocyclylene, or a bond, wherein the heterocyclylene contains 1-3 heteroatoms selected from N and O;

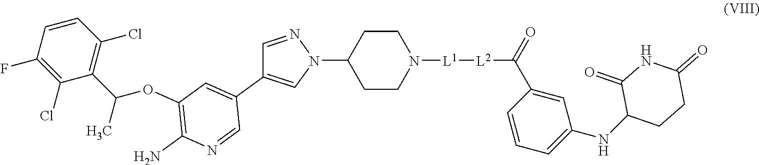

wherein:
L¹ is a bond; and
L² is 6- to 12-membered spiro heterocyclylene containing 1-3 heteroatoms selected from N and O.

Embodiment A16. A compound selected from the compounds of Table 1 and pharmaceutically acceptable salts thereof.

Embodiment A17. A pharmaceutical composition comprising the compound of any one of embodiments A1-A16, or a pharmaceutically acceptable salt thereof, and a pharmaceutically acceptable excipient.

Embodiment A18. A method of modulating Interleukin-1 Receptor-Associated Kinase 3 (IRAK3) comprising contacting IRAK3 with an effective amount of the compound of any one of embodiments A1-A16, or a pharmaceutically acceptable salt thereof, or the pharmaceutical composition of embodiment A17.

Embodiment A19. A method of treating cancer in a subject in need thereof, comprising administering to the subject an effective amount of the compound of any one of embodiments A1-A16, or a pharmaceutically acceptable salt thereof, or the pharmaceutical composition of embodiment A17, optionally wherein the cancer is selected from bladder cancer, breast cancer, esophageal cancer, colon cancer, head and neck cancer, kidney cancer, lung cancer, pancreatic cancer, prostate cancer, melanoma, and gastric cancer.

Embodiment A20. A method of enhancing immunity in a subject receiving a vaccine, comprising administering to the subject an effective amount of the compound of any one of embodiments AT-A16, or a pharmaceutically acceptable salt thereof, or the pharmaceutical composition of embodiment A17.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean ±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkylene” group refers to the same residues as alkyl, but having bivalency. Particular alkylene groups are those having from 1 to 10 carbon atoms (C₁-C₁₀ alkylene), typically from 1 to 8 carbons (C₁-C₈ alkylene) or, in some embodiments, from 1 to 6 (C₁-C₆ alkylene) or 1 to 3 (C₁-C₃ alkylene) carbon atoms. Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heterocyclylene” group refers to a divalent “heterocyclyl” group.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

An “alkoxy” group is —O-(alkyl), wherein alkyl is defined above.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (IA) or Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of Formula (IA) or Formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

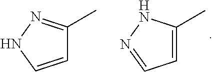

As readily understood by one skilled in the art, a wide variety of functional groups and other structures may exhibit tautomerism and all tautomers of compounds of Formula (IA) or Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an IRAK3 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein are compounds of Formula (IA):

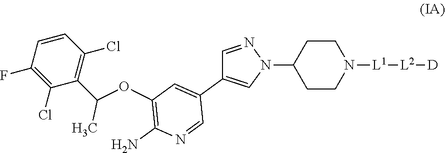

or a pharmaceutically acceptable salt thereof, wherein:
L¹ is —(CH₂CH₂O)ₙ— or a bond;
n is 1-10;
L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O;
D is

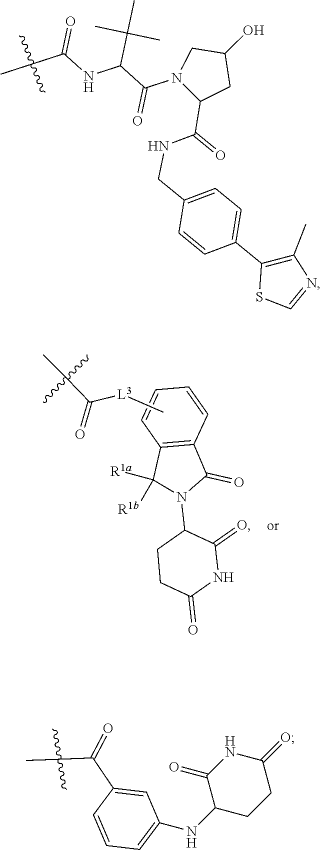

R^{1a} and R^{1b} are each H or are taken together to form an oxo; and
L³ is —CH₂O— or a bond.

In a further aspect, provided herein is a compound of Formula (I):

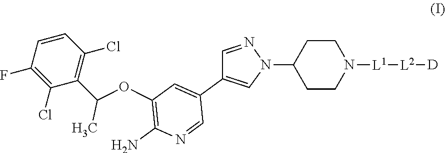

or a pharmaceutically acceptable salt thereof, wherein:
L¹ is —(CH₂CH₂O)ₙ— or a bond;
n is 1-10;
L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O;
D is

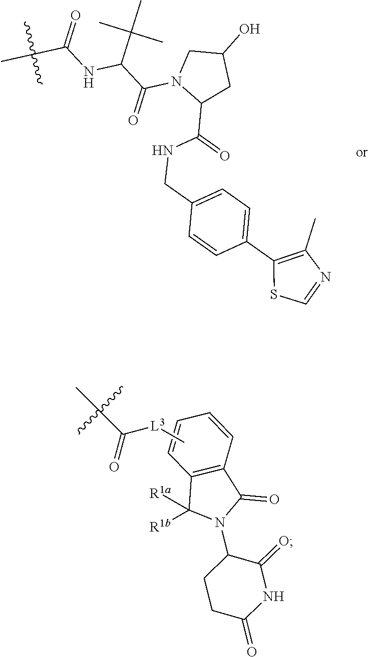

R^{1a} and R^{1b} are each H or are taken together to form an oxo; and
L³ is —CH₂O— or a bond.

In some embodiments, L¹ is —(CH₂CH₂O)ₙ— or a bond. In some embodiments, L¹ is —(CH₂CH₂O)ₙ—. In some embodiments, L¹ is a bond.

In some embodiments, n is 1-10. In some embodiments, n is 1-9. In some embodiments, n is 1-8. In some embodiments, n is 1-7. In some embodiments, n is 1-6. In some embodiments, n is 1-5. In some embodiments, n is 1-4. In some embodiments, n is 1-3. In some embodiments, n is 1-2. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O. In some embodiments, L² is C₁-C₃ alkylene, —(C₁-C₃ alkylene)N(H)—, 10- to 12-membered spiro heterocyclylene, or a bond, wherein the heterocyclylene contains 1-2 nitrogen atoms.

In some embodiments, L² is C₁-C₆ alkylene. In some embodiments, L² is C₁-C₃ alkylene. In some embodiments, L² is —CH₂—, —CH₂CH₂—, or —CH₂CH₂CH₂—. In some embodiments, L² is —CH₂—. In some embodiments, L² is —CH₂CH₂—. In some embodiments, L² is —CH₂CH₂CH₂—.

In some embodiments, L² is —(C₁-C₆ alkylene)N(H)—. In some embodiments, L² is —(C₁-C₃ alkylene)N(H)—. In some embodiments, L² is —CH₂N(H)—, —CH₂CH₂N(H), or —CH₂CH₂CH₂N(H)—. In some embodiments, L² is —CH₂N(H)—. In some embodiments, L² is —CH₂CH₂N(H)—. In some embodiments, L² is —CH₂CH₂CH₂N(H)—.

In some embodiments, L² is 6- to 12-membered spiro heterocyclylene, wherein the heterocyclylene contains 1-3 heteroatoms selected from N and O. In some embodiments, L² is 6- to 12-membered spiro heterocyclylene, wherein the heterocyclylene contains 1-2 heteroatoms selected from N and O. In some embodiments, L² is 10- to 12-membered spiro heterocyclylene, wherein the heterocyclylene contains 1-2 nitrogen atoms. In some embodiments, L² is 10- to 12-membered spiro heterocyclylene, wherein the heterocyclylene contains one nitrogen atom. In some embodiments, L² is 10- to 12-membered spiro heterocyclylene, wherein the heterocyclylene contains two nitrogen atoms. In some embodiments, L² is 10-membered spiro heterocyclylene, wherein the heterocyclylene contains one nitrogen atom. In some embodiments, L² is 11-membered spiro heterocyclylene, wherein the heterocyclylene contains one nitrogen atom.

In some embodiments, L² is a bond.

In some embodiments, L² is L² is —CH₂—, —CH₂CH₂CH₂—, —CH₂CH₂N(H)—,

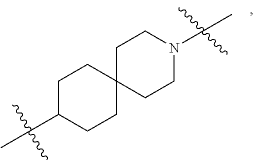 [SMILES: *C1CCC2(CC1)CCN(*)CC2]

or a bond.

In some embodiments, -L¹-L²- is:

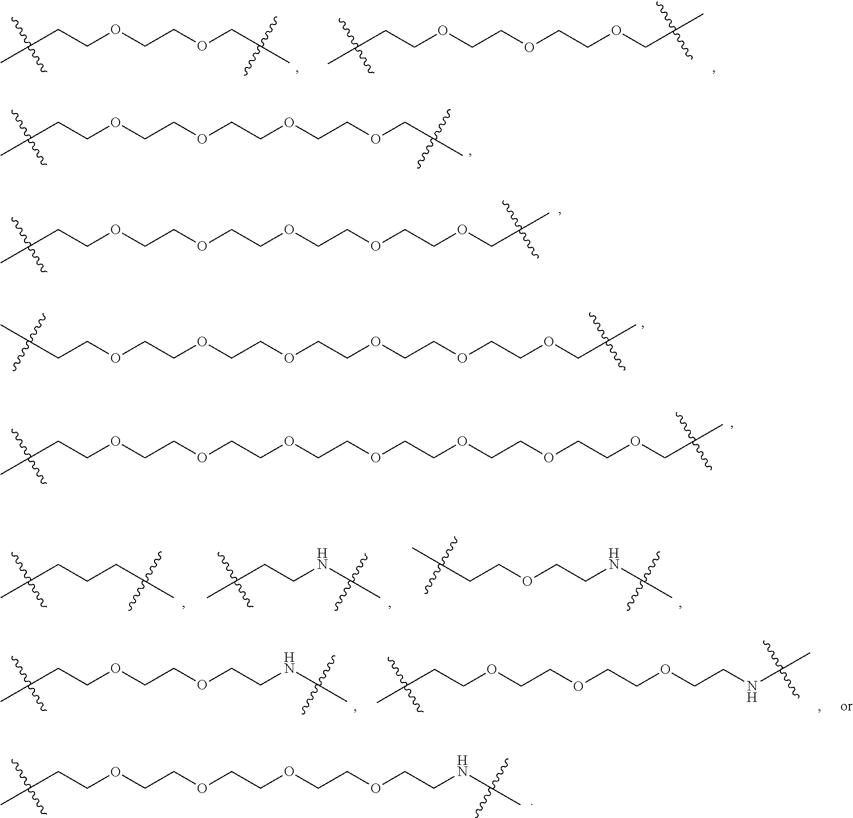

In some embodiments, -L¹-L²- is

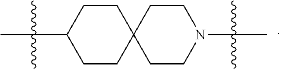 [SMILES: *C1CCC2(CC1)CCN(*)CC2]

In some embodiments, D is

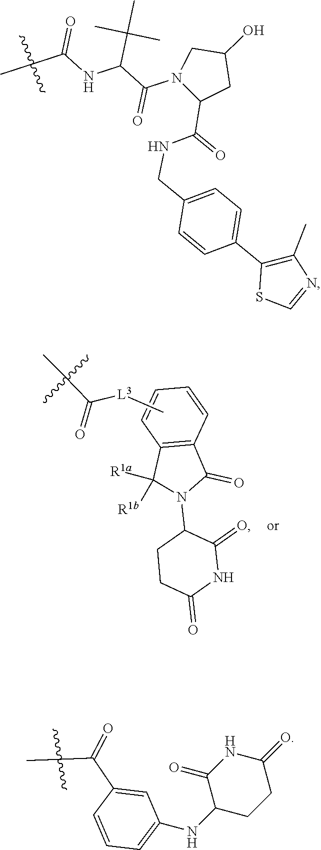

In some embodiments, D is

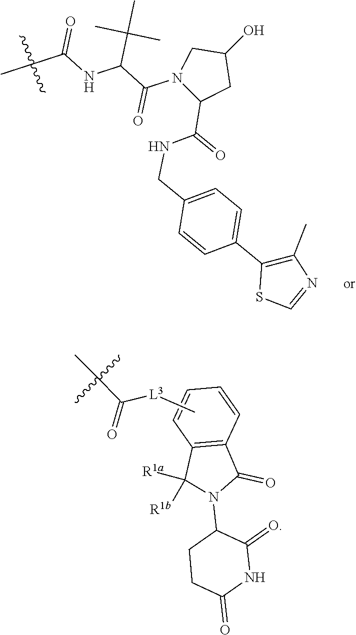

In some embodiments, D is

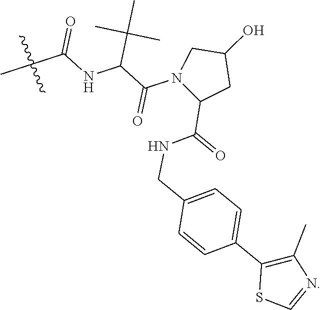 [SMILES: *C(=O)NC(C(=O)N1CC(O)CC1C(=O)NCc1ccc(-c2scnc2C)cc1)C(C)(C)C]

In some embodiments, D is

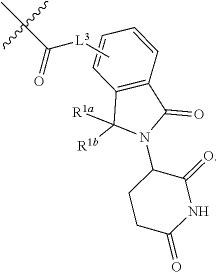

In some embodiments, D is

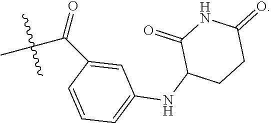 [SMILES: *C(=O)c1cccc(NC2CCC(=O)NC2=O)c1]

In some embodiments, R^{1a} and R^{1b} are each H or are taken together to form an oxo. In some embodiments, R^{1a} and R^{1b} are each H. In some embodiments, R^{1a} and R^{1b} are taken together to form an oxo.

In some embodiments, D is

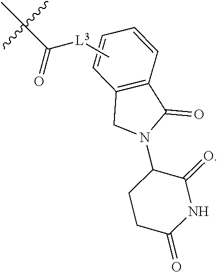

In some embodiments, D is

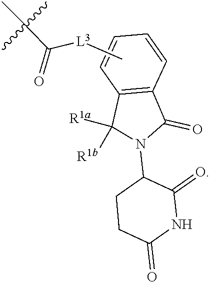

In some embodiments, D is:

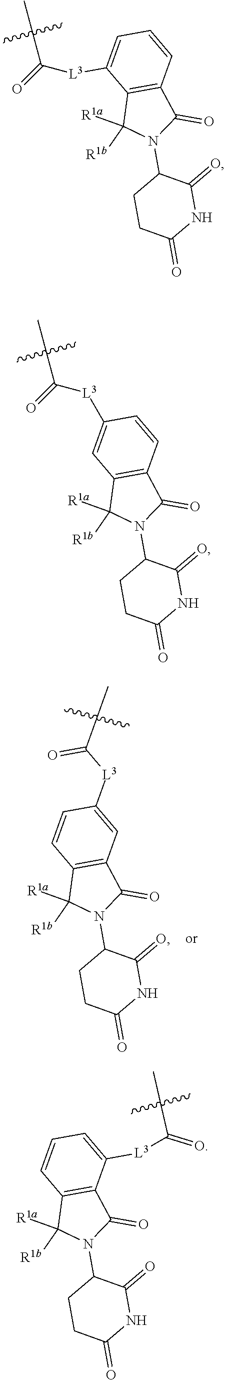

In some embodiments, L³ is —CH₂O— or a bond. In some embodiments, L³ is —CH₂O—. In some embodiments, L³ is a bond.

In some embodiments,

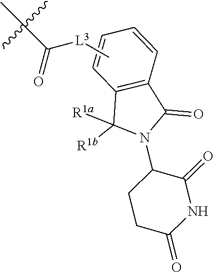

is:

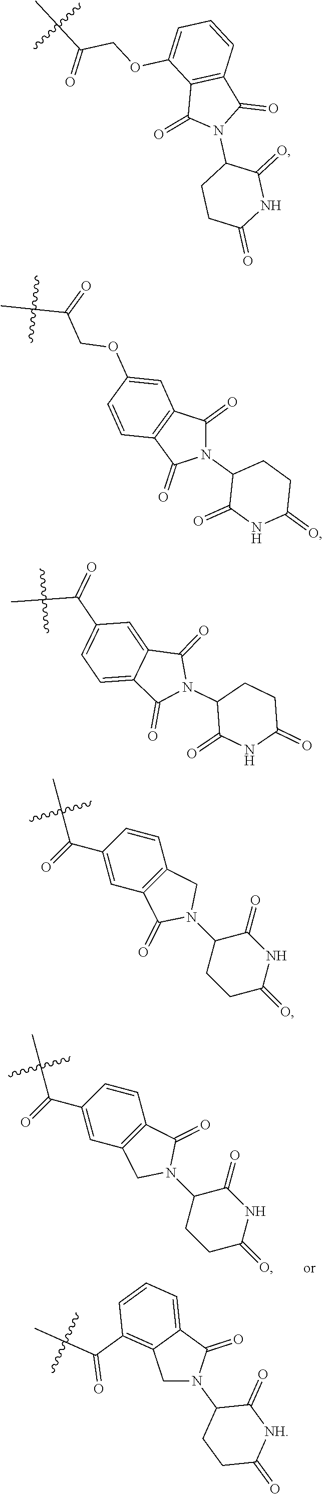 [SMILES: *C(=O)COc1cccc2c1C(=O)N(C1CCC(=O)NC1=O)C2=O ; *C(=O)COc1ccc2c(c1)C(=O)N(C1CCC(=O)NC1=O)C2=O ; *C(=O)c1ccc2c(c1)C(=O)N(C1CCC(=O)NC1=O)C2=O ; *C(=O)c1ccc2c(c1)C(=O)N(C1CCC(=O)NC1=O)C2 ; *C(=O)c1ccc2c(c1)CN(C1CCC(=O)NC1=O)C2=O ; *C(=O)c1cccc2c1CN(C1CCC(=O)NC1=O)C2=O]

In some embodiments, the compound of Formula (IA) or Formula (I) is a compound of Formula (Ia):

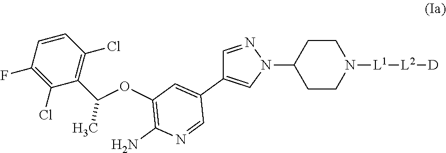

wherein L¹, L², and D are as described for Formula (IA) or Formula (I).

In some embodiments, the compound of Formula (IA) or Formula (I) is a compound of Formula (II):

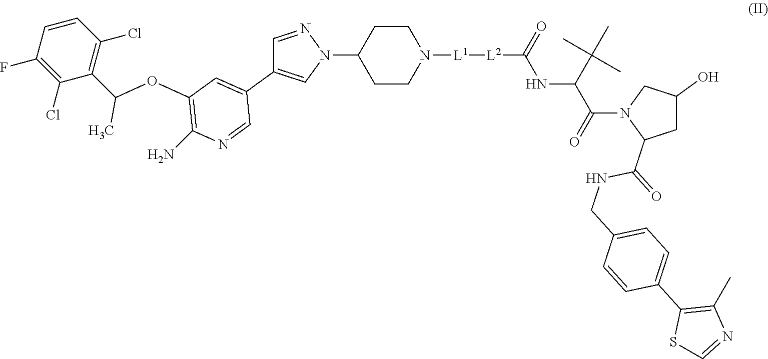

wherein L¹ is —(CH₂CH₂O)ₙ— or a bond; n is 1-10; and L² is C₁-C₆ alkylene.

In some embodiments, the compound of Formula (IA) or Formula (I) is a compound of Formula (IIa):

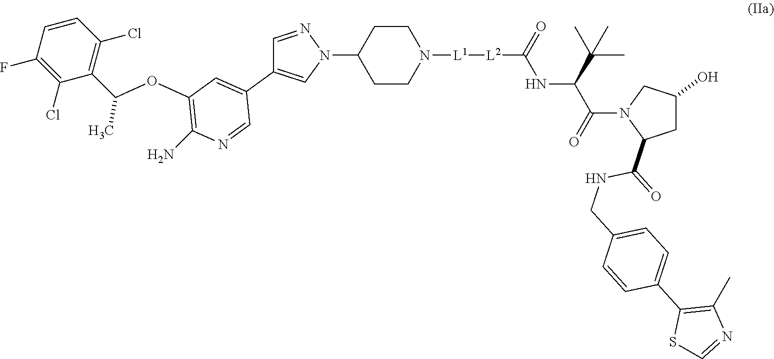

wherein L¹ is —(CH₂CH₂O)ₙ— or a bond; n is 1-10; and L² is C₁-C₆ alkylene.

In some embodiments, the compound of Formula (IA) or Formula (I) is a compound of Formula (III):

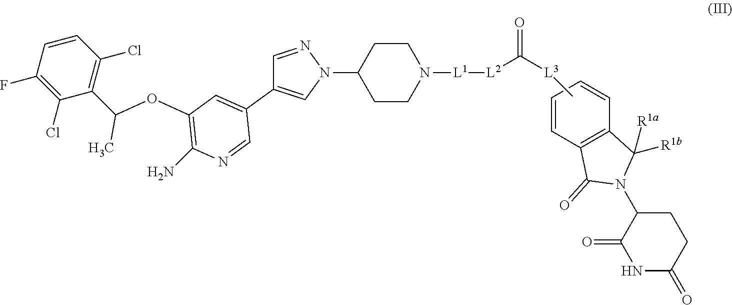

wherein L¹ —(CH₂CH₂O)ₙ— or a bond; n is 1-10; and L² is —(C₁-C₆ alkylene)-N(H)—, 6- to 12-membered spiro heterocyclylene, or a bond, wherein the heterocyclylene contains 1-3 heteroatoms selected from N and O.

In some embodiments, the compound of Formula (IA) or Formula (I) is a compound of Formula (IIIa):

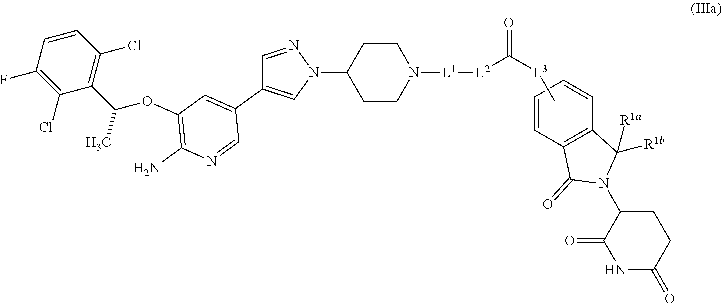

wherein L¹ —(CH₂CH₂O)ₙ— or a bond; n is 1-10; and L² is —(C₁-C₆ alkylene)-N(H)—, 6- to 12-membered spiro heterocyclylene, or a bond, wherein the heterocyclylene contains 1-3 heteroatoms selected from N and O.

In some embodiments, the compound of Formula (IA) or Formula (I) is a compound of Formula (IV) or (IVa):

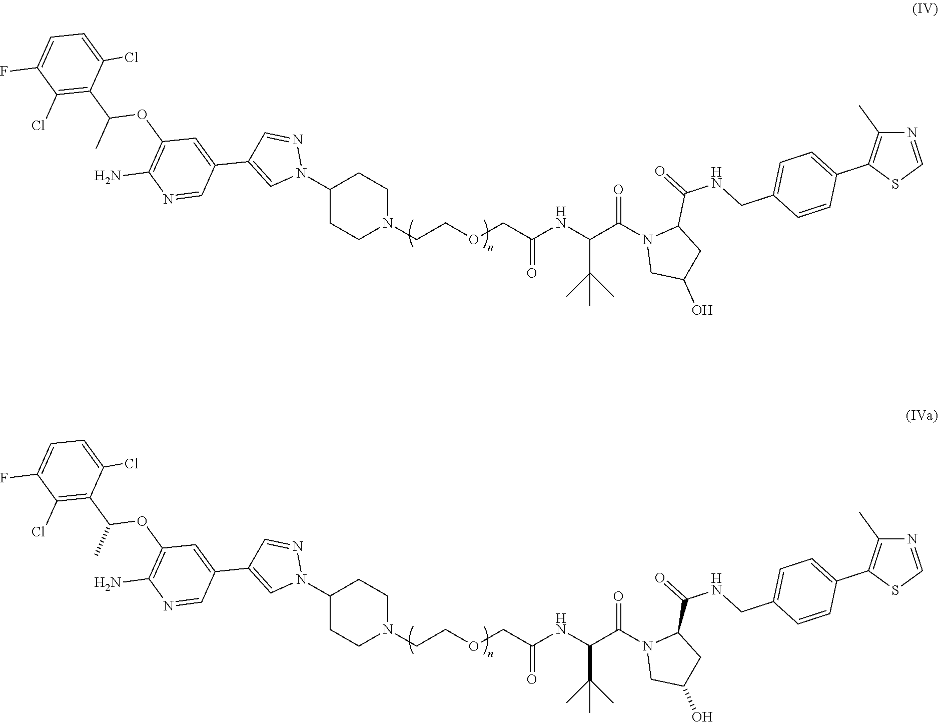

wherein n is as described for Formula (IA) or Formula (I). In some embodiments, n is 2-7.

In some embodiments, the compound of Formula (IA) or Formula (I) is a compound of Formula (V), (Va), (Vb), or (Vc):

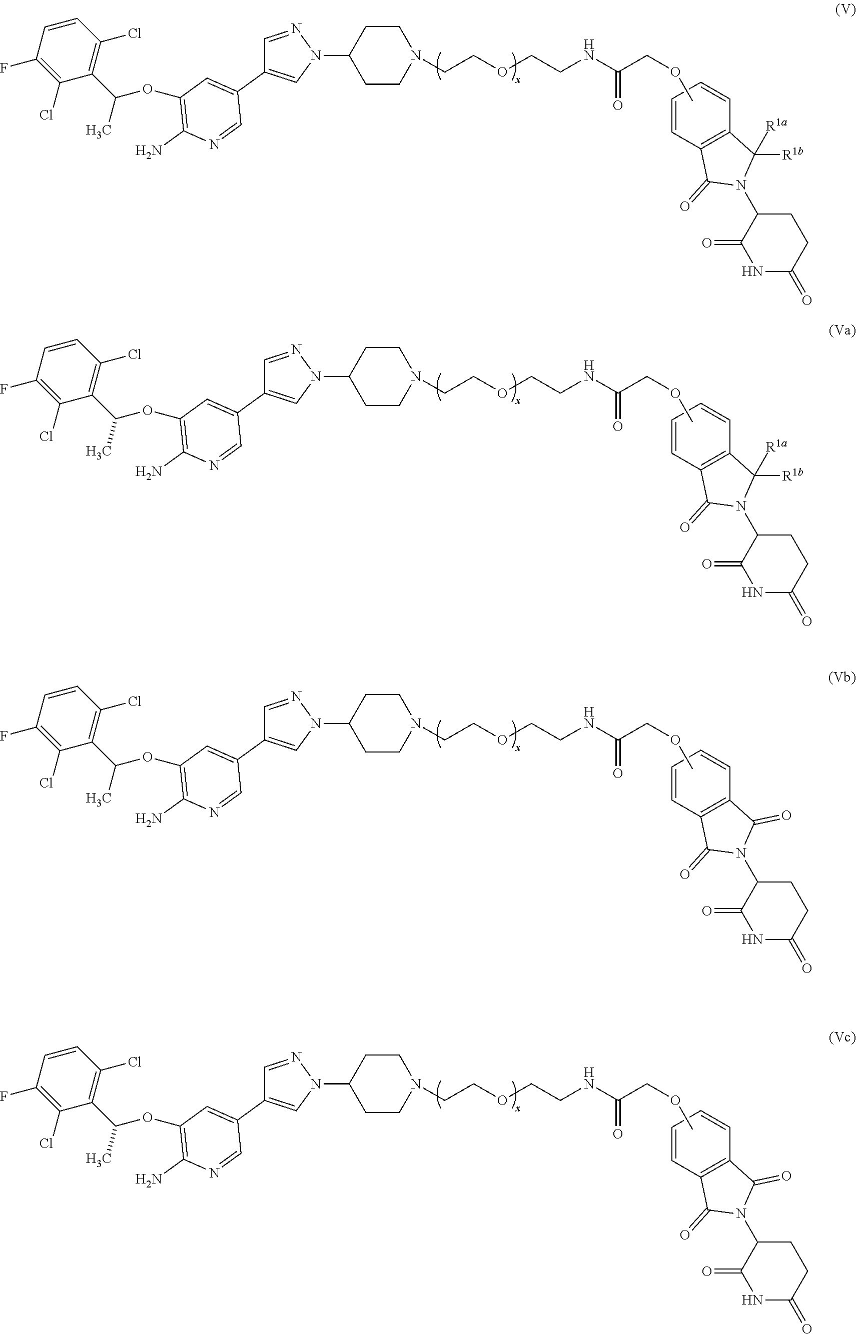

wherein x is 0-10; and R^{1a} and R^{1b} are as described for Formula (IA) or Formula (I). In some embodiments, x is n as described for Formula (IA) or Formula (I). In some embodiments, x is 1-10. In some embodiments, x is 1-4. In some embodiments, x is 0.

In some embodiments, the compound of Formula (IA) or Formula (I) is a compound of Formula (VI) or (VIa):

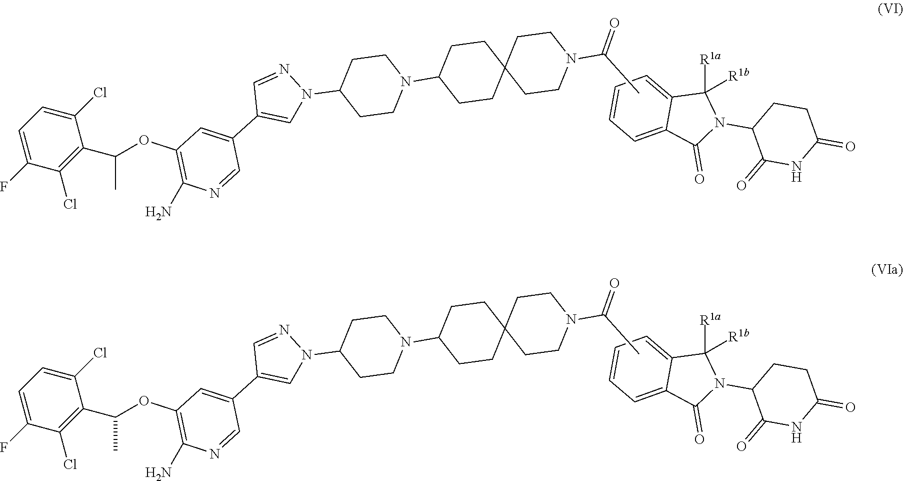

wherein R^{1a} and R^{1b} are as described for Formula (IA) or Formula (I).

In some embodiments, the compound of Formula (IA) or Formula (I) is a compound of the compound is of Formula (VII) or (VIIa):

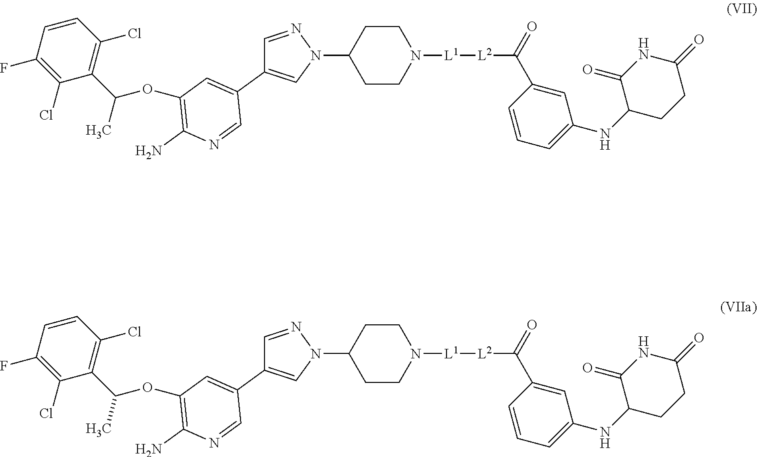

wherein L¹ is a bond; and L² is 6- to 12-membered spiro heterocyclylene containing 1-3 heteroatoms selected from N and O. In some embodiments, L² is 11-membered spiro heterocyclylene containing 1-2 nitrogen atoms.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to L¹ of Formula (IA) or Formula (I) may be combined with every description, variation, embodiment, or aspect of L², L³, D, R^{1a}, R^{1b}, and n the same as if each and every combination were specifically and individually listed. It is also understood that all descriptions, variations, embodiments, or aspects of Formula (IA) or Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (IA) or Formula (I), where applicable, apply equally to any of the formulae as detailed herein, such as Formulae (Ia), (II), (IIa), (III), (IIIa), (IV), (IVa), (V), (Va), (Vb), (Vc), (VI), (VIa), (VII), and (VIIa), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | |
|---|---|
| No. | Structure |
| 1 | 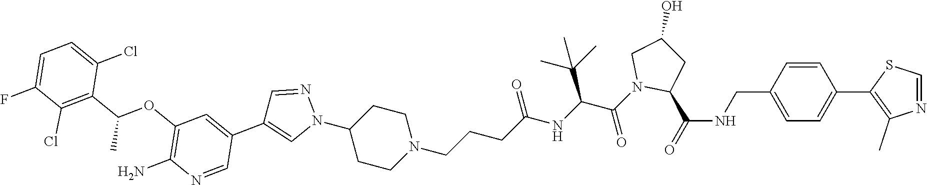 [SMILES: Cc1ncsc1-c1ccc(CNC(=O)[C@@H]2C[C@@H](O)CN2C(=O)[C@@H](NC(=O)CCCN2CCC(n3cc(-c4cnc(N)c(O[C@H](C)c5c(Cl)ccc(F)c5Cl)c4)cn3)CC2)C(C)(C)C)cc1] |
| 2 | 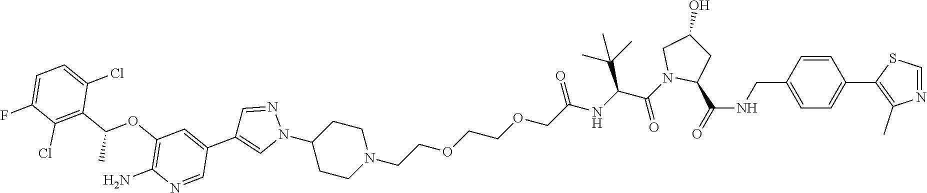 [SMILES: Cc1ncsc1-c1ccc(CNC(=O)[C@@H]2C[C@@H](O)CN2C(=O)[C@@H](NC(=O)COCCOCCN2CCC(n3cc(-c4cnc(N)c(O[C@H](C)c5c(Cl)ccc(F)c5Cl)c4)cn3)CC2)C(C)(C)C)cc1] |
| 3 | 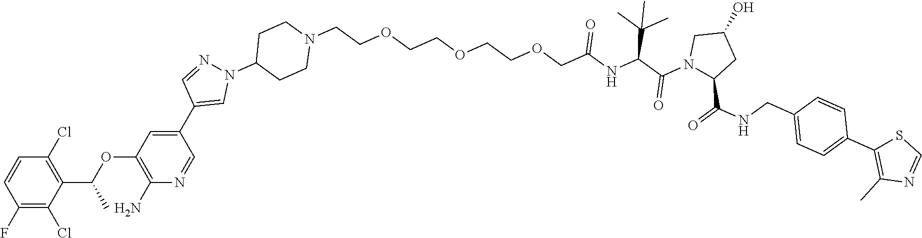 [SMILES: Cc1ncsc1-c1ccc(CNC(=O)[C@@H]2C[C@@H](O)CN2C(=O)[C@@H](NC(=O)COCCOCCOCCN2CCC(n3cc(-c4cnc(N)c(O[C@H](C)c5c(Cl)ccc(F)c5Cl)c4)cn3)CC2)C(C)(C)C)cc1] |
| 4 | 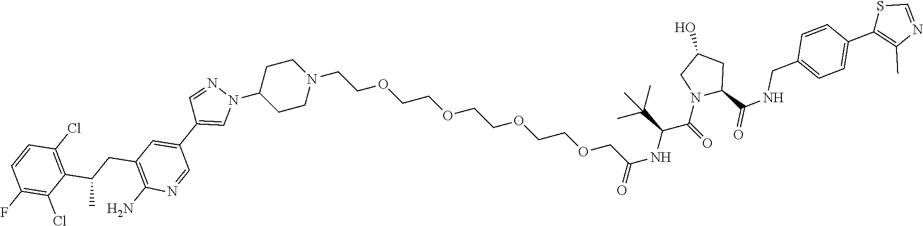 [SMILES: Cc1ncsc1-c1ccc(CNC(=O)[C@@H]2C[C@@H](O)CN2C(=O)[C@@H](NC(=O)COCCOCCOCCOCCN2CCC(n3cc(-c4cnc(N)c(C[C@H](C)c5c(Cl)ccc(F)c5Cl)c4)cn3)CC2)C(C)(C)C)cc1] |
| 5 | 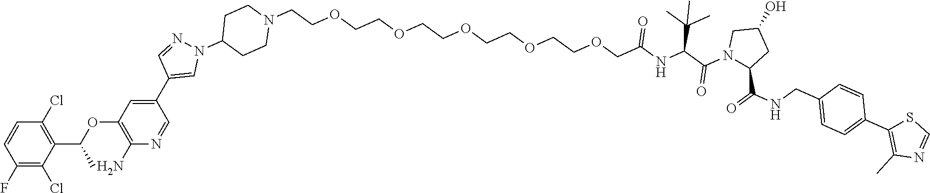 [SMILES: Cc1ncsc1-c1ccc(CNC(=O)[C@@H]2C[C@@H](O)CN2C(=O)[C@@H](NC(=O)COCCOCCOCCOCCOCCN2CCC(n3cc(-c4cnc(N)c(O[C@H](C)c5c(Cl)ccc(F)c5Cl)c4)cn3)CC2)C(C)(C)C)cc1] |
| 6 | 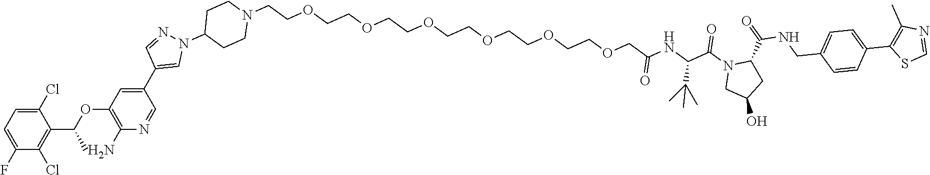 [SMILES: Cc1ncsc1-c1ccc(CNC(=O)[C@@H]2C[C@@H](O)CN2C(=O)[C@@H](NC(=O)COCCOCCOCCOCCOCCOCCN2CCC(n3cc(-c4cnc(N)c(O[C@H](C)c5c(Cl)ccc(F)c5Cl)c4)cn3)CC2)C(C)(C)C)cc1] |
| 7 | 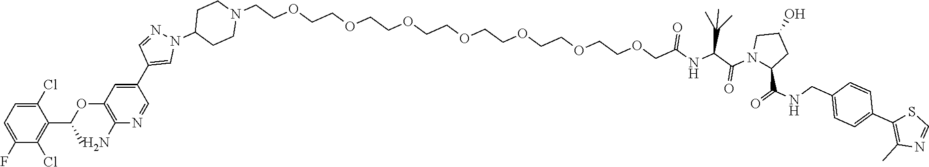 [SMILES: Cc1ncsc1-c1ccc(CNC(=O)[C@@H]2C[C@@H](O)CN2C(=O)[C@@H](NC(=O)COCCOCCOCCOCCOCCOCCOCCN2CCC(n3cc(-c4cnc(N)c(O[C@H](C)c5c(Cl)ccc(F)c5Cl)c4)cn3)CC2)C(C)(C)C)cc1] |
| 8 | 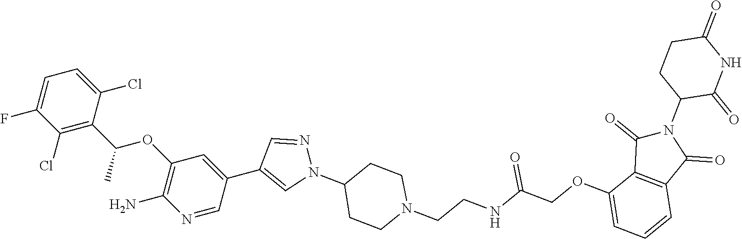 [SMILES: C[C@@H](Oc1cc(-c2cnn(C3CCN(CCNC(=O)COc4cccc5c4C(=O)N(C4CCC(=O)NC4=O)C5=O)CC3)c2)cnc1N)c1c(Cl)ccc(F)c1Cl] |
| 9 | 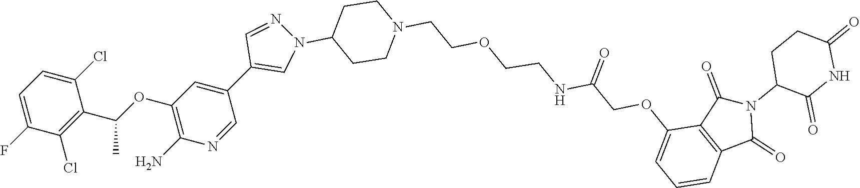 [SMILES: C[C@@H](Oc1cc(-c2cnn(C3CCN(CCOCCNC(=O)COc4cccc5c4C(=O)N(C4CCC(=O)NC4=O)C5=O)CC3)c2)cnc1N)c1c(Cl)ccc(F)c1Cl] |
| 10 | 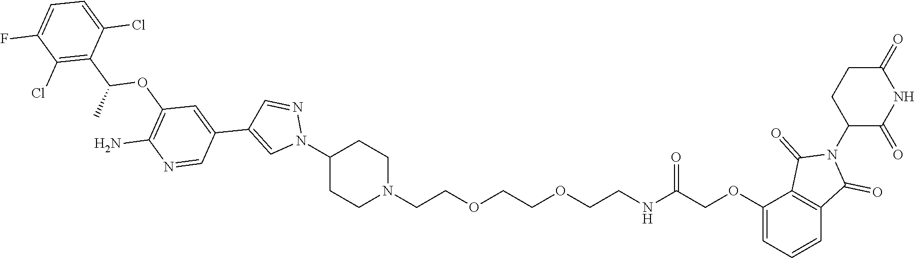 [SMILES: C[C@@H](Oc1cc(-c2cnn(C3CCN(CCOCCOCCNC(=O)COc4cccc5c4C(=O)N(C4CCC(=O)NC4=O)C5=O)CC3)c2)cnc1N)c1c(Cl)ccc(F)c1Cl] |
| 11 | 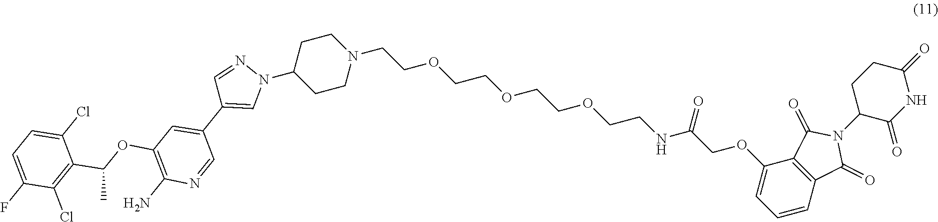 [SMILES: C[C@@H](Oc1cc(-c2cnn(C3CCN(CCOCCOCCOCCNC(=O)COc4cccc5c4C(=O)N(C4CCC(=O)NC4=O)C5=O)CC3)c2)cnc1N)c1c(Cl)ccc(F)c1Cl] |
| 12 | 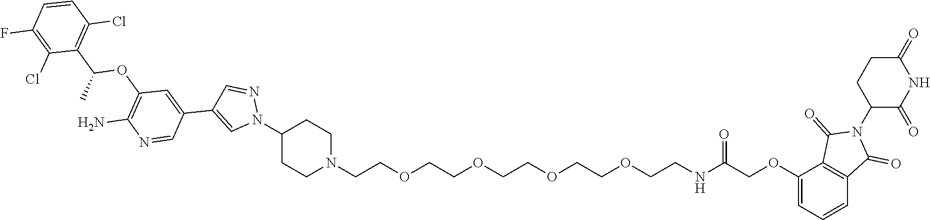 [SMILES: C[C@@H](Oc1cc(-c2cnn(C3CCN(CCOCCOCCOCCOCCNC(=O)COc4cccc5c4C(=O)N(C4CCC(=O)NC4=O)C5=O)CC3)c2)cnc1N)c1c(Cl)ccc(F)c1Cl] |
| 13 | 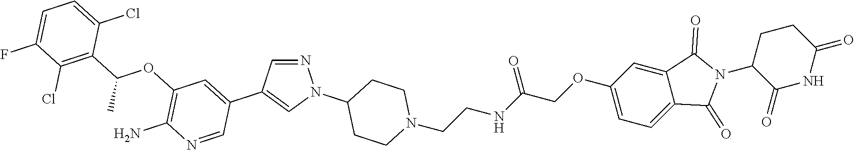 [SMILES: C[C@@H](Oc1cc(-c2cnn(C3CCN(CCNC(=O)COc4ccc5c(c4)C(=O)N(C4CCC(=O)NC4=O)C5=O)CC3)c2)cnc1N)c1c(Cl)ccc(F)c1Cl] |
| 14 | 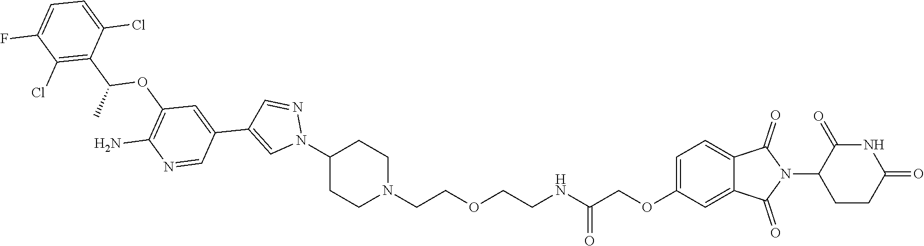 [SMILES: C[C@@H](Oc1cc(-c2cnn(C3CCN(CCOCCNC(=O)COc4ccc5c(c4)C(=O)N(C4CCC(=O)NC4=O)C5=O)CC3)c2)cnc1N)c1c(Cl)ccc(F)c1Cl] |
| 15 | 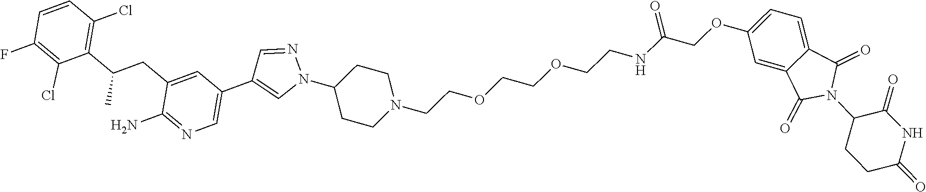 [SMILES: C[C@@H](Cc1cc(-c2cnn(C3CCN(CCOCCOCCNC(=O)COc4ccc5c(c4)C(=O)N(C4CCC(=O)NC4=O)C5=O)CC3)c2)cnc1N)c1c(Cl)ccc(F)c1Cl] |
| 16 | 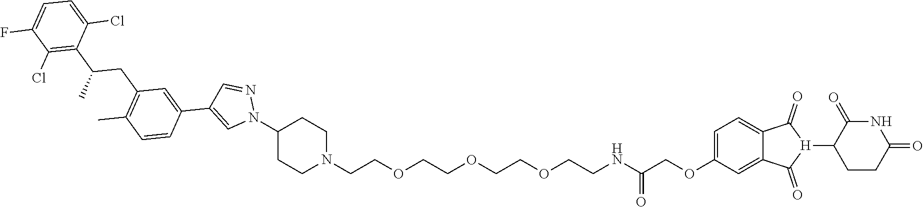 |
| 17 | 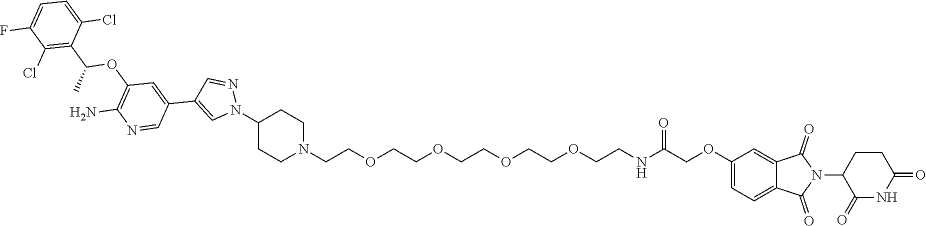 [SMILES: C[C@@H](Oc1cc(-c2cnn(C3CCN(CCOCCOCCOCCOCCNC(=O)COc4ccc5c(c4)C(=O)N(C4CCC(=O)NC4=O)C5=O)CC3)c2)cnc1N)c1c(Cl)ccc(F)c1Cl] |
| 18 | 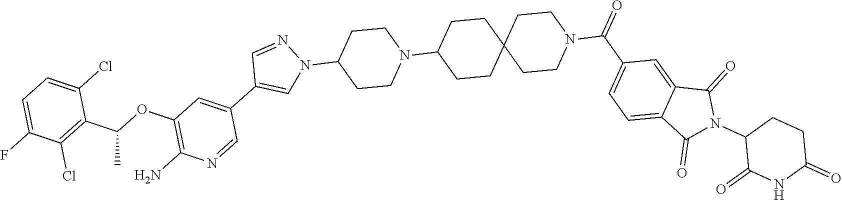 [SMILES: C[C@@H](Oc1cc(-c2cnn(C3CCN(C4CCC5(CC4)CCN(C(=O)c4ccc6c(c4)C(=O)N(C4CCC(=O)NC4=O)C6=O)CC5)CC3)c2)cnc1N)c1c(Cl)ccc(F)c1Cl] |
| 19 | 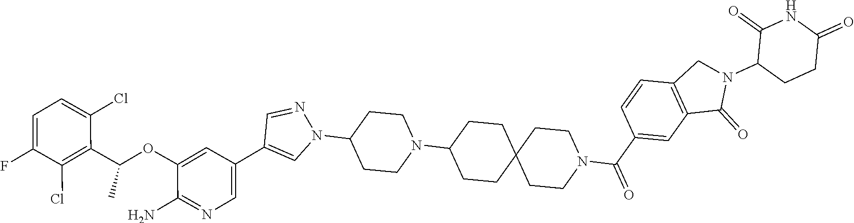 [SMILES: C[C@@H](Oc1cc(-c2cnn(C3CCN(C4CCC5(CC4)CCN(C(=O)c4ccc6c(c4)C(=O)N(C4CCC(=O)NC4=O)C6)CC5)CC3)c2)cnc1N)c1c(Cl)ccc(F)c1Cl] |
| 20 | 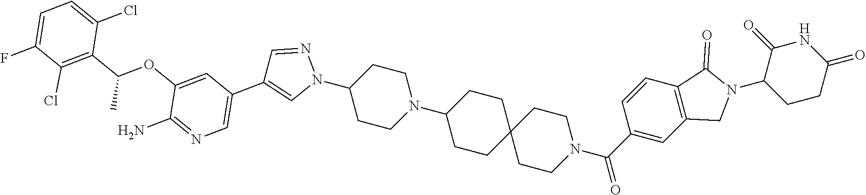 [SMILES: C[C@@H](Oc1cc(-c2cnn(C3CCN(C4CCC5(CC4)CCN(C(=O)c4ccc6c(c4)CN(C4CCC(=O)NC4=O)C6=O)CC5)CC3)c2)cnc1N)c1c(Cl)ccc(F)c1Cl] |
| 21 | 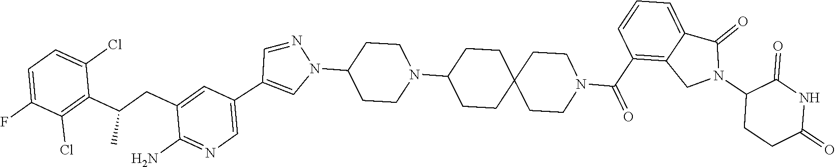 [SMILES: C[C@@H](Cc1cc(-c2cnn(C3CCN(C4CCC5(CC4)CCN(C(=O)c4cccc6c4CN(C4CCC(=O)NC4=O)C6=O)CC5)CC3)c2)cnc1N)c1c(Cl)ccc(F)c1Cl] |
| 22 | 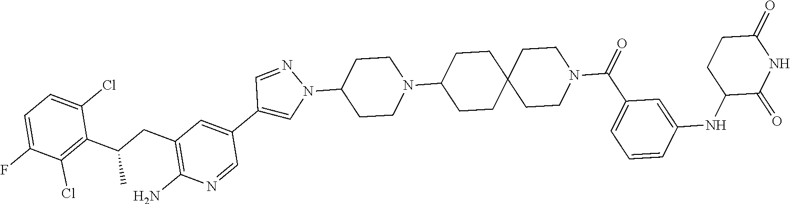 [SMILES: C[C@@H](Cc1cc(-c2cnn(C3CCN(C4CCC5(CC4)CCN(C(=O)c4cccc(NC6CCC(=O)NC6=O)c4)CC5)CC3)c2)cnc1N)c1c(Cl)ccc(F)c1Cl] |

or a pharmaceutically acceptable salt thereof.

It is understood that in the present description, combinations of substituents and/or variables of the depicted formulae are permissible only if such contributions result in stable compounds.

Furthermore, all compounds of Formula (IA) or Formula (I) that exist in free base or acid form can be converted to their pharmaceutically acceptable salts by treatment with the appropriate inorganic or organic base or acid by methods known to one skilled in the art. Salts of the compounds of Formula (IA) or Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (IA) or Formula (I) can be prepared as outlined in Scheme 1, as well as in the examples set forth herein. It should be noted that one skilled in the art would know how to modify the procedures set forth in the illustrative schemes and examples to arrive at the desired products.

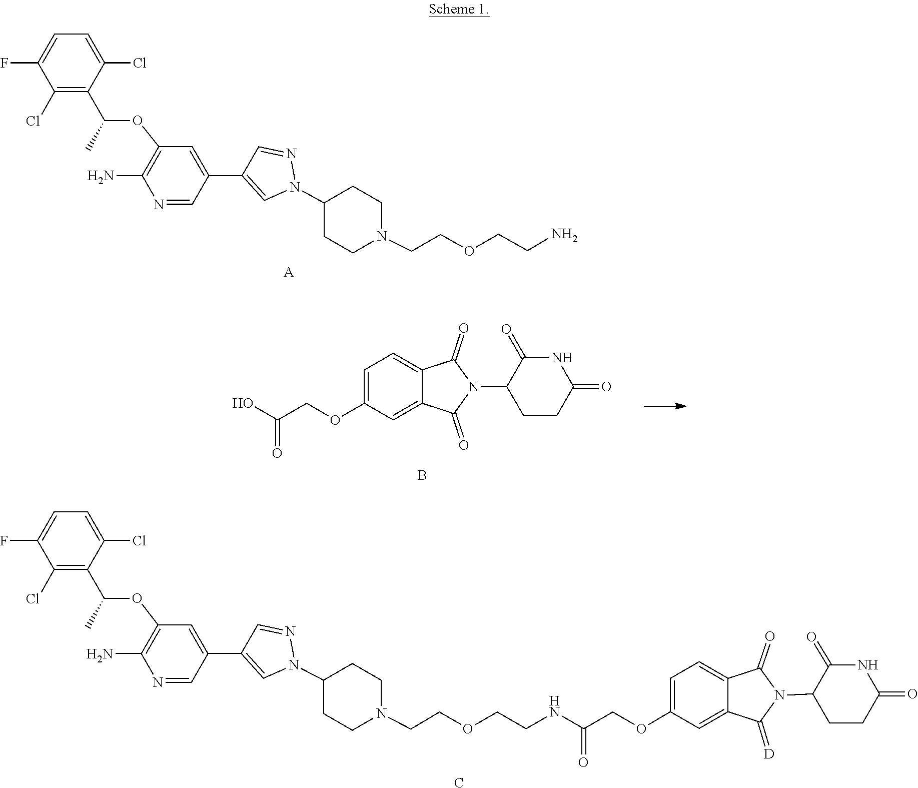

Scheme 1 illustrates an approach to the synthesis of compounds exemplified by C. Intermediates A and B can be coupled by various amide coupling reagents. For example, simple treatment of B with HATU and DIPEA in DMF, followed by the addition of A affords compounds exemplified by C.

### Methods of Use

Embodiments of the present disclosure provide a method for modulating IRAK3 in a subject in need thereof, the method comprising administering to the subject an effective amount of a compound of Formula (IA) or Formula (I). Modulation (e.g., inhibition or activation) of IRAK3 can be assessed and demonstrated by a wide variety of ways known in the art. Kits and commercially available assays can be utilized for determining whether and to what degree IRAK3 has been modulated (e.g., inhibited or activated).

In one aspect, provided herein is a method of modulating IRAK3 comprising contacting IRAK3 with an effective amount of a compound of Formula (IA) or Formula (I) or any embodiment or variation thereof. In some embodiments, the compound of Formula (IA) or Formula (I) inhibits IRAK3. In other embodiments, the compound of Formula (IA) or Formula (I) activates IRAK3. In some embodiments, the compound of Formula (IA) or Formula (I) causes degradation of IRAK3.

In some embodiments, provided herein is a method for targeting IRAK3 for degradation comprising contacting IRAK3 with an effective amount of a compound of Formula (IA) or Formula (I) or any embodiment or variation thereof.

In some embodiments, a compound of Formula (IA) or Formula (I) modulates the activity of IRAK3 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (IA) or Formula (I) modulates the activity of IRAK3 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

Also provided in certain embodiments of the present disclosure is a method for degrading IRAK3 in a subject in need thereof, the method comprising administering to the subject an effective amount of a compound of Formula (IA) or Formula (I). Degradation of IRAK3 can be assessed and demonstrated by a wide variety of ways known in the art. Kits and commercially available assays, including cell-based assays, can be utilized for determining whether and to what degree IRAK3 has been degraded.

In one aspect, provided herein is a method of degrading IRAK3 comprising contacting IRAK3 with an effective amount of a compound of Formula (IA) or Formula (I) or any embodiment or variation thereof. In some embodiments, the compound of Formula (IA) or Formula (I) partially degrades IRAK3. In some embodiments, the compound of Formula (IA) or Formula (I) fully degrades IRAK3.

In some embodiments, a compound of Formula (IA) or Formula (I) degrades IRAK3 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (IA) or Formula (I) degrades IRAK3 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating cancer in a subject in need thereof, comprising administering to the subject an effective amount of a compound of Formula (IA) or Formula (I). In some embodiments, provided herein is a method for preventing cancer in a subject in need thereof, comprising administering to the subject an effective amount of a compound of Formula (IA) or Formula (I). Non-limiting examples of cancer include bladder cancer, breast cancer, esophageal cancer, colon cancer, head and neck cancer, kidney cancer, lung cancer, pancreatic cancer, prostate cancer, melanoma, and gastric cancer.

In some embodiments, administering a compound of Formula (IA) or Formula (I) to a subject that is predisposed to cancer prevents the subject from developing any symptoms of the cancer (such as tumor growth or metastasis). In some embodiments, administering a compound of Formula (IA) or Formula (I) to a subject that does not yet display symptoms of cancer prevents the subject from developing any symptoms of the cancer. In some embodiments, administering a compound of Formula (IA) or Formula (I) to a subject in need thereof diminishes the extent of the cancer in the subject. In some embodiments, administering a compound of Formula (IA) or Formula (I) to a subject in need thereof stabilizes the cancer (prevents or delays the worsening of the cancer). In some embodiments, administering a compound of Formula (IA) or Formula (I) to a subject in need thereof delays the occurrence or recurrence of the cancer. In some embodiments, administering a compound of Formula (IA) or Formula (I) to a subject in need thereof slows the progression of the cancer. In some embodiments, administering a compound of Formula (IA) or Formula (I) to a subject in need thereof provides a partial remission of the cancer. In some embodiments, administering a compound of Formula (IA) or Formula (I) to a subject in need thereof provides a total remission of the cancer. In some embodiments, administering a compound of Formula (IA) or Formula (I) to a subject in need thereof decreases the dose of one or more other medications required to treat the cancer. In some embodiments, administering a compound of Formula (IA) or Formula (I) to a subject in need thereof enhances the effect of another medication used to treat the cancer. In some embodiments, administering a compound of Formula (IA) or Formula (I) to a subject in need thereof delays the progression of the cancer. In some embodiments, administering a compound of Formula (IA) or Formula (I) to a subject in need thereof increases the quality of life of the subject having cancer. In some embodiments, administering a compound of Formula (IA) or Formula (I) to a subject in need thereof prolongs survival of a subject having cancer.

In one aspect, provided herein is method of preventing a subject that is predisposed to cancer from developing cancer, the method comprising administering a compound of Formula (IA) or Formula (I) to the subject.

In some aspects, provided herein is a method of diminishing the extent of cancer in a subject, the method comprising administering a compound of Formula (IA) or Formula (I) to the subject. In some embodiments, provided herein is a method of stabilizing cancer in a subject, the method comprising administering a compound of Formula (IA) or Formula (I) to the subject. In some embodiments, the method prevents the worsening of the cancer.

In another aspect, provided herein is a method of delaying the occurrence or recurrence of cancer in a subject, the method comprising administering a compound of Formula (IA) or Formula (I) to the subject.

In some embodiments, provided herein is a method of slowing the progression of cancer in a subject, the method comprising administering a compound of Formula (IA) or Formula (I) to the subject. In some embodiments, the method provides a partial remission of the cancer. In some embodiments, the method provides a total remission of the cancer.

In further aspects, provided herein is a method of decreasing the dose of one or more other medications required to treat cancer in a subject, the method comprising administering a compound of Formula (IA) or Formula (I) to the subject. In some embodiments, provided herein is a method of enhancing the effect of another medication used to treat cancer in a subject, the method comprising administering a compound of Formula (IA) or Formula (I) to the subject.

Also provided here is a method of delaying the progression of cancer in a subject, the method comprising administering a compound of Formula (IA) or Formula (I) to the subject. In some embodiments, the method increases the quality of life of the subject having cancer. In some embodiments, the method prolongs survival of the subject having cancer.

In some embodiments, compounds of Formula (IA) or Formula (I) are useful for treating a cancer selected from bladder cancer, breast cancer, esophageal cancer, colon cancer, head and neck cancer, kidney cancer, lung cancer, pancreatic cancer, prostate cancer, melanoma, and gastric cancer.

In some embodiments, provided herein is a method to enhance immunity in a subject receiving a vaccine, comprising administering to the subject an effective amount of a compound of Formula (IA) or Formula (I). In some embodiments, the compound of Formula (IA) or Formula (I) is administered to the subject prior to the administration of a vaccine. In some embodiments, the compound of Formula (IA) or Formula (I) is administered to the subject simultaneously to the administration of a vaccine. In some embodiments, the compound of Formula (IA) or Formula (I) is administered to the subject following the administration of a vaccine. In some embodiments, the compound of Formula (IA) or Formula (I) is formulated as a component of the vaccine. In some embodiments, the compound of Formula (IA) or Formula (I) is formulated separately from the vaccine.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (IA) or Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (IA) or Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of Formula (IA) or Formula (I) administered will depend on such factors as the solubility of the active component, the formulation used and the route of administration.

In some embodiments, a compound of Formula (IA) or Formula (I) is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (IA) or Formula (I).

In a particular embodiment, provided herein are unit dosage formulations comprising about 0.1 mg or 100 mg of a compound of Formula (IA) or Formula (I).

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a compound of Formula (IA) or Formula (I).

A compound of Formula (IA) or Formula (I) can be administered once, twice, three, four or more times daily. In a particular embodiment, doses of 100 mg or less are administered as a once daily dose and doses of more than 100 mg are administered twice daily in an amount equal to one half of the total daily dose.

A compound of Formula (IA) or Formula (I) can be administered orally for reasons of convenience. In one embodiment, when administered orally, a compound of Formula (IA) or Formula (I) is administered with a meal and water. In another embodiment, the compound of Formula (IA) or Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (IA) or Formula (I) without an additional carrier, excipient or vehicle.

In another embodiment, provided herein are compositions comprising an effective amount of a compound of Formula (IA) or Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (IA) or Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (IA) or Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (IA) or Formula (I) can be delayed or prolonged by proper formulation. For example, a slowly soluble pellet of the compound of Formula (IA) or Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (IA) or Formula (I) in oily or emulsified vehicles that allow it to disperse slowly in the serum.

### EXEMPLARY EMBODIMENTS

The present disclosure is further described by the following embodiments. The features of each of the embodiments are combinable with any of the other embodiments where appropriate and practical.

Embodiment 1. A compound of Formula (IA):

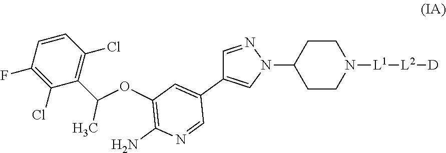

or a pharmaceutically acceptable salt thereof, wherein:
L¹ is —(CH₂CH₂O)ₙ— or a bond;
n is 1-10;
L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O;
D is

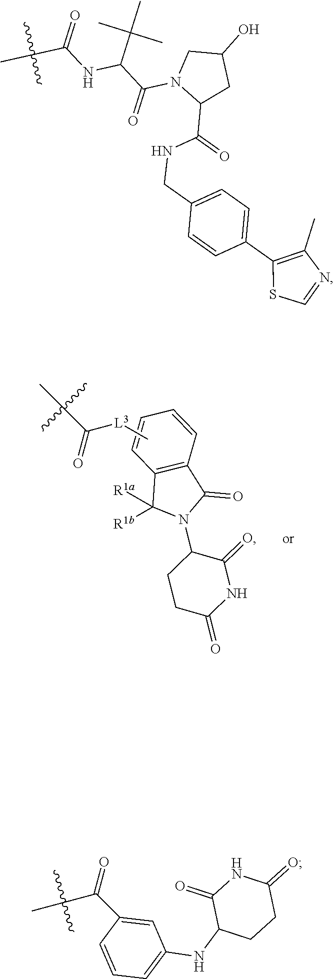

R^{1a} and R^{1b} are each H or are taken together to form an oxo; and
L³ is —CH₂O— or a bond.

Embodiment 2. The compound of embodiment 1, or a pharmaceutically acceptable salt thereof, wherein the compound is of Formula (I):

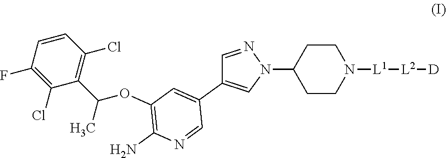

wherein:
L¹ is —(CH₂CH₂O)ₙ— or a bond;
n is 1-10;
L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O;
D is

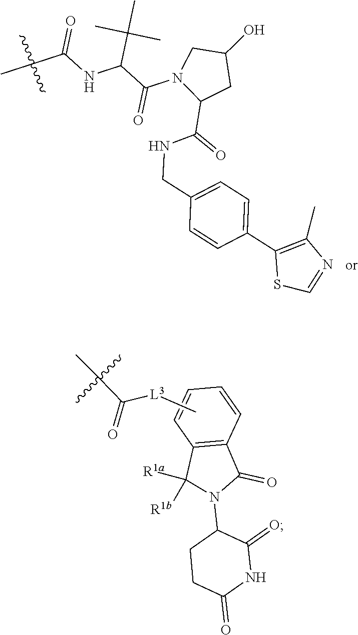

R^{1a} and R^{1b} are each H or are taken together to form an oxo; and
L³ is —CH₂O— or a bond.

Embodiment 3. The compound of embodiment 1 or 2, or a pharmaceutically acceptable salt thereof, wherein:
L¹ is —(CH₂CH₂O)ₙ—.

Embodiment 4. The compound of embodiment 3, or a pharmaceutically acceptable salt thereof, wherein:
n is 1-7.

Embodiment 5. The compound of embodiment 1 or 2, or a pharmaceutically acceptable salt thereof, wherein:
L¹ is a bond.

Embodiment 6. The compound of any one of embodiments 1-5, or a pharmaceutically acceptable salt thereof, wherein:
L² is C₁-C₃ alkylene, —(C₁-C₃ alkylene)N(H)—, 10- to 12-membered spiro heterocyclylene, or a bond, wherein the heterocyclylene contains 1-2 nitrogen atoms.

Embodiment 7. The compound of embodiment 6, or a pharmaceutically acceptable salt thereof, wherein:
L² is —CH₂—, —CH₂CH₂CH₂—, —CH₂CH₂N(H)—,

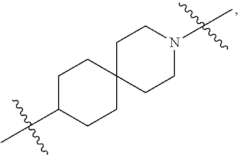 [SMILES: *C1CCC2(CC1)CCN(*)CC2]

or a bond.

Embodiment 8. The compound of any one of embodiments 1-7, or a pharmaceutically acceptable salt thereof, wherein:
-L¹-L²- is

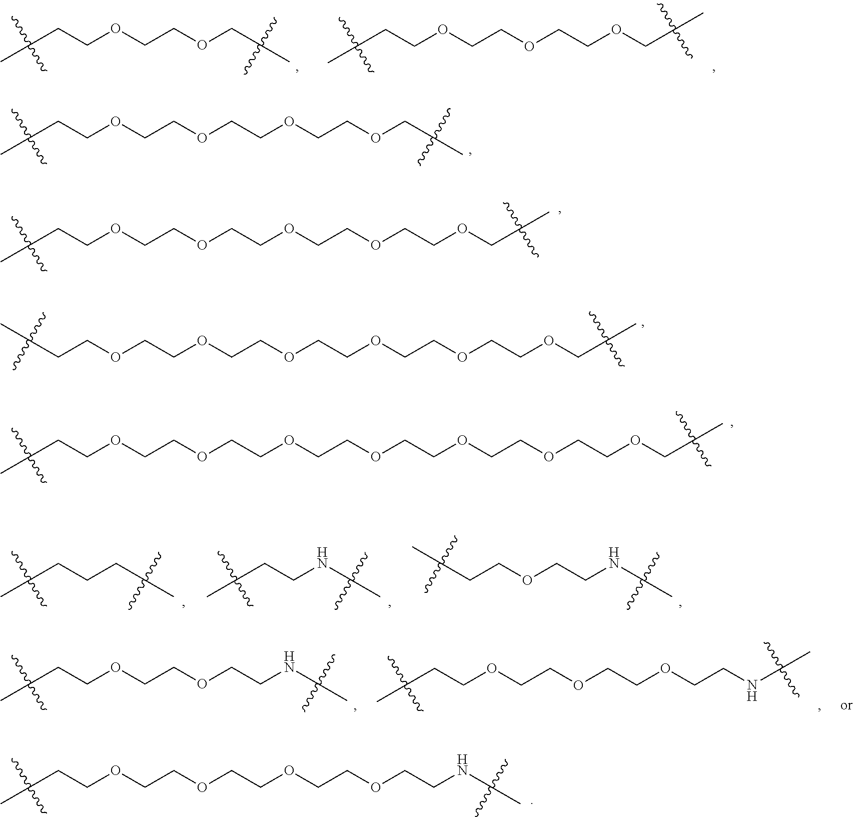

Embodiment 9. The compound of any one of embodiments 1-7, or a pharmaceutically acceptable salt thereof, wherein:
-L¹-L²- is

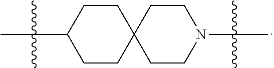 [SMILES: *C1CCC2(CC1)CCN(*)CC2]

Embodiment 10. The compound of any one of embodiments 1-9, or a pharmaceutically acceptable salt thereof, wherein:
D is

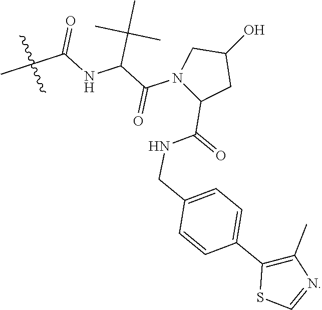 [SMILES: *C(=O)NC(C(=O)N1CC(O)CC1C(=O)NCc1ccc(-c2scnc2C)cc1)C(C)(C)C]

Embodiment 11. The compound of any one of embodiments 1-9, or a pharmaceutically acceptable salt thereof, wherein:
D is

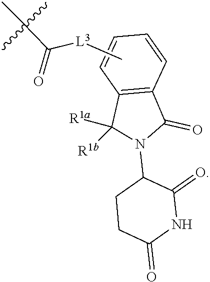

Embodiment 12. The compound of embodiment 11, or a pharmaceutically acceptable salt thereof, wherein:
R^{1a} and R^{1b} are each H.

Embodiment 13. The compound of embodiment 11, or a pharmaceutically acceptable salt thereof, wherein:
R^{1a} and R^{1b} are taken together to form an oxo.

Embodiment 14. The compound of any one of embodiments 11-13, or a pharmaceutically acceptable salt thereof, wherein:
L³ is —CH₂O—.

Embodiment 15. The compound of any one of embodiments 11-13, or a pharmaceutically acceptable salt thereof, wherein:
L³ is a bond.

Embodiment 16. The compound of any one of embodiments 11-15, or a pharmaceutically acceptable salt thereof, wherein:

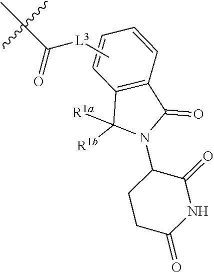

is

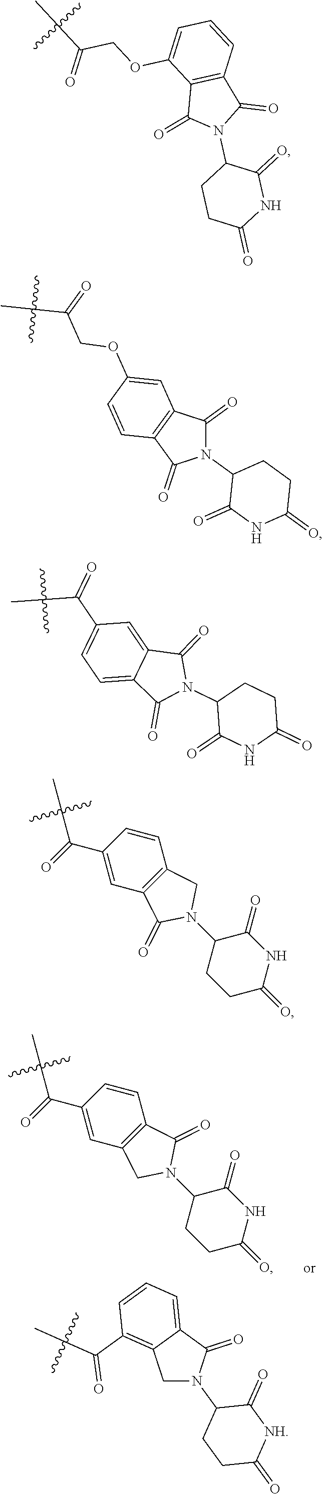 [SMILES: *C(=O)COc1cccc2c1C(=O)N(C1CCC(=O)NC1=O)C2=O ; *C(=O)COc1ccc2c(c1)C(=O)N(C1CCC(=O)NC1=O)C2=O ; *C(=O)c1ccc2c(c1)C(=O)N(C1CCC(=O)NC1=O)C2=O ; *C(=O)c1ccc2c(c1)C(=O)N(C1CCC(=O)NC1=O)C2 ; *C(=O)c1ccc2c(c1)CN(C1CCC(=O)NC1=O)C2=O ; *C(=O)c1cccc2c1CN(C1CCC(=O)NC1=O)C2=O]

Embodiment 17. The compound of any one of embodiments 1-9, or a pharmaceutically acceptable salt thereof, wherein:
D is

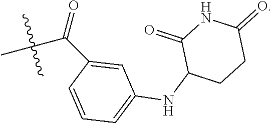 [SMILES: *C(=O)c1cccc(NC2CCC(=O)NC2=O)c1]

Embodiment 18. The compound of any one of embodiments 1-9, or a pharmaceutically acceptable salt thereof, wherein the compound is of Formula (II):

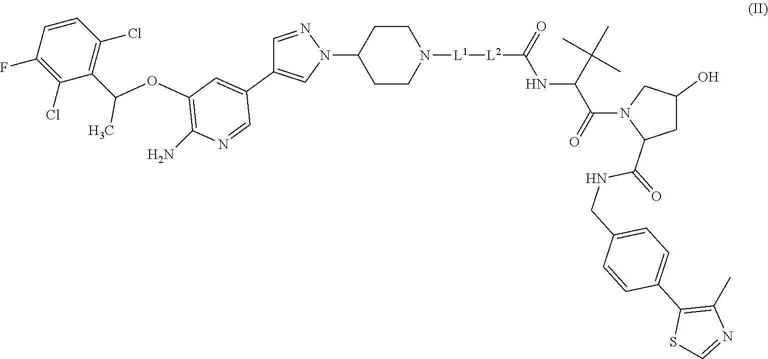

wherein:
L¹ is —(CH₂CH₂O)ₙ— or a bond;
n is 1-10; and
L² is C₁-C₆ alkylene.

Embodiment 19. The compound of any one of embodiments 1-9 and 11-16, or a pharmaceutically acceptable salt thereof, wherein the compound is of Formula (III):

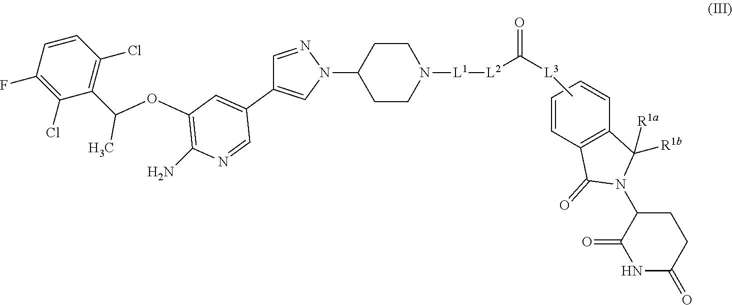

wherein:
L¹ —(CH₂CH₂O)ₙ— or a bond;
n is 1-10; and
L² is —(C₁-C₆ alkylene)-N(H)—, 6- to 12-membered spiro heterocyclylene, or a bond, wherein the heterocyclylene contains 1-3 heteroatoms selected from N and O.

Embodiment 20. The compound of any one of embodiments 1-9 and 17, or a pharmaceutically acceptable salt thereof, wherein the compound is of Formula (VII):

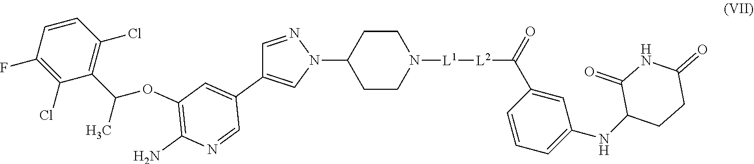

L¹ is a bond; and
L² is 6- to 12-membered Spiro heterocyclylene containing 1-3 heteroatoms selected from N and O.

Embodiment 21. A compound selected from the compounds of Table 1 and pharmaceutically acceptable salts thereof.

Embodiment 22. A pharmaceutical composition comprising the compound of any one of embodiments 1-21, or a pharmaceutically acceptable salt thereof, and a pharmaceutically acceptable excipient.

Embodiment 23. A method of modulating Interleukin-1 Receptor-Associated Kinase 3 (IRAK3) comprising contacting IRAK3 with an effective amount of the compound of any one of embodiments 1-21, or a pharmaceutically acceptable salt thereof, or the pharmaceutical composition of embodiment 22.

Embodiment 24. A method of treating cancer in a subject in need thereof, comprising administering to the subject an effective amount of the compound of any one of embodiments 1-21, or a pharmaceutically acceptable salt thereof, or the pharmaceutical composition of embodiment 22.

Embodiment 25. The method of embodiment 24, wherein the cancer is selected from bladder cancer, breast cancer, esophageal cancer, colon cancer, head and neck cancer, kidney cancer, lung cancer, pancreatic cancer, prostate cancer, melanoma, and gastric cancer.

Embodiment 26. A method of enhancing immunity in a subject receiving a vaccine, comprising administering to the subject an effective amount of the compound of any one of embodiments 1-21, or a pharmaceutically acceptable salt thereof, or the pharmaceutical composition of embodiment 22.

Embodiment 27. The method of embodiment 26, wherein the subject is administered the vaccine prior to, concurrently with, or after administration of the compound of any one of embodiments 1-21, or a pharmaceutically acceptable salt thereof, or the pharmaceutical composition of embodiment 22.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

| | |
|---|---|
| DCE | dichloroethane |
| DCM | dichloromethane |
| DIEA/DIPEA | diisopropylethylamine |
| DMA | dimethylacetamide |
| DMAP | 4-dimethylaminopyridine |
| DMF | dimethyl formamide |
| DMSO | dimethyl sulfoxide |
| EA or EtOAc | ethyl acetate |
| FA | formic acid |
| h | hour(s) |
| HATU | O-(7-azabenzotriazol-1-y1)- |
| | N,N,N′,N′- |
| | tetramethyluronium |
| | hexafluorophosphate |
| hex | hexanes |
| IBX | 2-iodoxybenzoic acid |
| HPLC | high pressure liquid chromatography |
| LCMS or LC/MS | liquid chromatography mass spectrometry |
| MeCN | acetonitrile |
| MeOH | methanol |
| min | minute(s) |
| PyAOP | (7-azabenzotriazol-1- |
| | yloxy)tripyrrolidinophosphonium |
| | hexafluorophosphate |
| rt | retention time |
| TFA | trifluoroacetic acid |
| THF | tetrahydrofuran |

### Synthetic Examples

### Analytical Methods

LC/MS Method 1. Column: Luna C18(2) 50×3 mm, 3 um. Temperature: 45° C., Flow: 2 mL/min, Run time: 2 min. Mobile phase conditions: Initial 95% H₂O 0.1% FA/5% MeCN 0.1% FA, linear gradient to 95% MeCN 0.1% FA over 1 min then hold for 1 minute at 95% CH₃CN 0.1% FA.]

LC/MS Method 2. Waters Acquity UPLC system. Column: ACQUIty UPLC BEH C18 1.7 mM (2.1×50 mm). Modifier: Formic Acid. Mobile Phase: Water—0.1% formic acid (A) and Acetonitrile—0.1% formic acid (B). Flow rate: 0.8 mL/min. Gradient: 5% B to 95% B in 1.5 min, holding at 95% B for 0.5 min, 95% B to 5% B in 0.1 min. Detector 1 UV 214 nm and 254 nm

LC/MS Method 3. SunFire C18 75×4.6 mm, 3.5 um. Temperature: 45° C., Flow: 1.5 mL/min, Run time: 6 min. Mobile phase conditions: Initial 95% H2O+0.1% FA/5% MeCN+0.1% FA then linear gradient to 95% MeCN for 4 min then hold for 2 min at 95% MeCN.

LC/MS Method 4. Luna C18(2) 50×3 mm, 3 um. Temperature: 45° C., Flow: 1.5 mL/min, Run time: 2.5 min. Mobile phase conditions: Initial 95% H2O 0.1% FA/5% MeCN 0.1% FA, linear gradient to 95% MeCN 0.1% FA over 1.3 min then hold for 1.2 minute at 95% MeCN 0.1% FA.

LC/MS Method 5. Luna C18 50×3 mm 3 um. Temperature: 45 C, Flow: 1.2 mL/min, run time: 5 min. Mobile phase conditions: Initial 95% H2O 0.05% TFA 5% CH3CN then linear gradient to 5% H2O 0.05% TFA 95% CH3CN for 3.5 min then hold at 95% CH3CN for 1.5 min.

LC/MS Method 6. Gemini C18 4.6×50 mm, 3 μm. Temperature: 45° C., Flow: 1.5 mL/min, Run time: 6 min. Mobile phase conditions: Initial 95% NH₄HCO₃/5% MeCN with 1 min equilibration, linear gradient to 95% MeCN over 3.5 min then hold for 2.5 minute.

### Example I-1. Preparation of (R)-4-(4-(4-(6-amino-5-(1-(2,6-dichloro-3-fluorophenyl)ethoxy)pyridin-3-yl)-1H-pyrazol-1-yl)piperidin-1-yl)butanoic acid (Intermediate I-1)

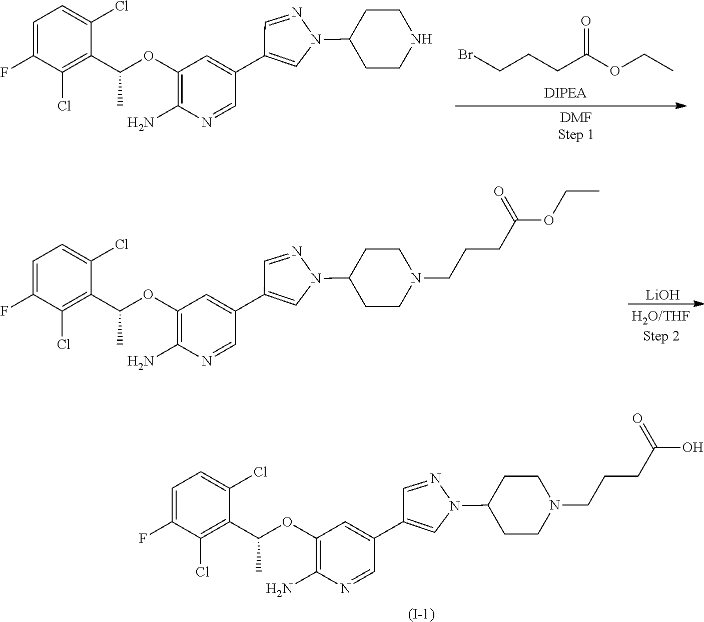

Step 1. To a solution of 5-[1-(4-piperidyl)pyrazol-4-yl]-3-[(1R)-1-(2,6-dichloro-3-fluoro-phenyl)ethoxy]pyridin-2-amine (125 mg, 0.2800 mmol) and ethyl 4-bromobutanoate (56.85 mg, 0.2900 mmol) in DMF (0.56 mL) and DMSO (0.1481 mL) at room temperature was added DIPEA (120.87 μL, 0.6900 mmol). The resulting solution was stirred at room temperature overnight. LCMS showed 91% conversion. The mixture was directly loaded on C18 for purification by reverse phase chromatography, eluting with 5% to 100% MeOH in EtOAc in 83% yield.

LCMS method 1: retention time: 1.040 min, 99.9% purity at 215 nm [M+H]⁺=564.2.

Step 2. To a solution of ethyl ethyl (R)-4-(4-(4-(6-amino-5-(1-(2,6-dichloro-3-fluorophenyl)ethoxy)pyridin-3-yl)-1H-pyrazol-1-yl)piperidin-1-yl)butanoate (130 mg, 0.2300 mmol) in THF (1 mL) and water (1 mL) was added LiOH (55.2 mg, 2.3 mmol). The resulting solution was stirred at room temperature overnight. LCMS showed complete conversion. The reaction mixture was concentrated and purified using reverse phase column chromatography to afford the desired product in 85% yield (105 mg).

LCMS method 5: retention time: 1.936 min, 98.3% purity at 215 nm [M+H]⁺=536.2, [M+2H]/2⁺=269.6.

### Example I-2. Preparation of 2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl 4-methylbenzenesulfonate (Intermediate I-2)

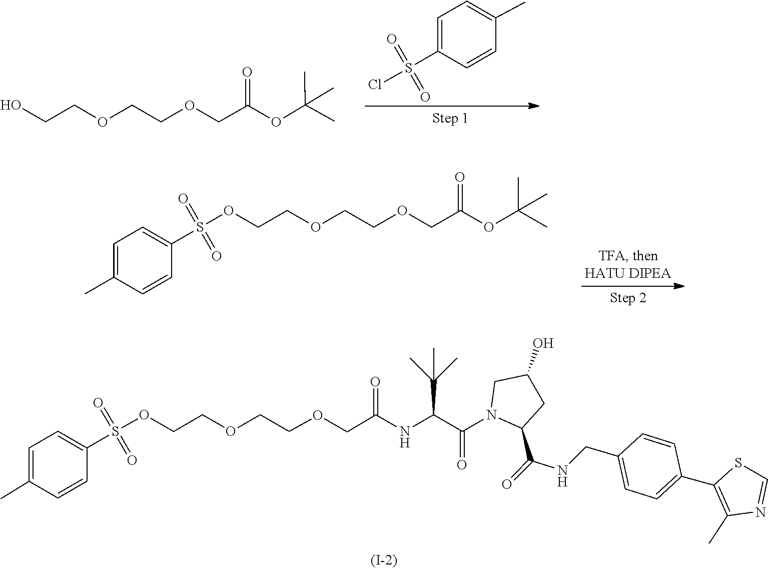

Step 1. To a solution of tert-butyl 2-(2-(2-hydroxyethoxy)ethoxy)acetate (1 g, 4.54 mmol) in dry DCM (18.16 mL) at 0° C. were added triethylamine (1265 μL, 9.08 mmol), 4-methylbenzenesulfonyl chloride (1.3 g, 6.81 mmol) and DMAP (55.47 mg, 0.4500 mmol). The reaction mixture was stirred overnight while the ice-water bath warmed to room temperature. HPLC showed complete conversion. The reaction was quenched with aqueous NH₄Cl and extracted 3× with EtOAc. The organics were washed with brine, dried over Na₂SO₄, concentrated under reduced pressure, and purified over normal phase column to afford 1.56 g of the desired product in 88% yield as a light yellow solid.

Step 2. To a solution of tert-butyl 2-(2-(2-(tosyloxy)ethoxy)ethoxy)acetate (1.56 g, 4.17 mmol) in DCM (7.7 mL) at room temperature was added TFA (3.8 mL, 47 mmol) dropwise. The reaction mixture was stirred at room temperature for 2 h. HPLC analysis showed complete conversion. The solvent was evaporated to dryness to afford the crude acid as a light yellow oil. To a solution of crude acid in DMF (8.3 mL) at room temperature were added DIPEA (2.2 mL, 12.5 mmol) and HATU (1.82 g, 4.79 mmol). The resulting solution was stirred at room temperature for 10 minutes, then (2S,4R)-1-((S)-2-amino-3,3-dimethylbutanoyl)-4-hydroxy-N-(4-(4-methylthiazol-5-yl)benzyl)pyrrolidine-2-carboxamide HCl salt (2.05 g, 4.39 mmol) was added in one portion. The reaction mixture was stirred at room temperature overnight. HPLC analysis showed complete conversion. The reaction mixture was purified over reverse phase column chromatography to give 2 g of the desired product in 61.6% yield as a white solid.

¹H NMR (400 MHz, DMSO-d₆) δ ppm 0.92 (s, 9H), 1.88-1.96 (m, 1H), 2.01-2.07 (m, 1H), 2.40 (s, 3H), 2.43 (s, 3H), 3.48-3.56 (m, 4H), 3.59-3.68 (m, 4H), 3.93 (s, 2H), 4.11-4.14 (m, 2H), 4.28 (d, J=5.3 Hz, 1H), 4.34-4.39 (m, 2H), 4.41-4.46 (m, 1H), 4.56 (d, J=9.6 Hz, 1H), 5.15 (d, J=3.5 Hz, 1H), 7.38-7.41 (m, 5H), 7.46 (d, J=7.8 Hz, 2H), 7.77 (d, J=8.3 Hz, 2H), 8.55-8.61 (m, 1H), 8.98 (s, 1H).

LCMS Method 3: retention time: 3.660 min, 93.2% purity at 215 nm [M+H]⁺=731.2.

### Example I-3. Preparation of (R)-20-(4-(4-(6-amino-5-(1-(2,6-dichloro-3-fluorophenyl)ethoxy)pyridin-3-yl)-1H-pyrazol-1-yl)piperidin-1-yl)-3,6,9,12,15,18-hexaoxaicosanoic acid (Intermediate I-3)

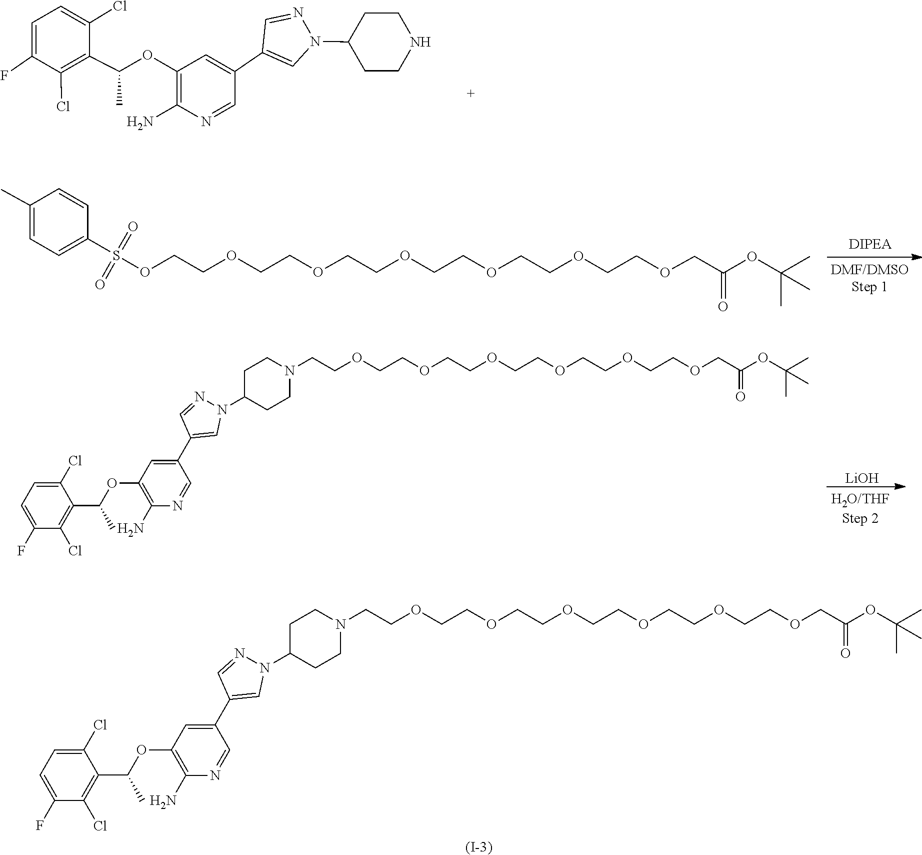 [SMILES: C[C@@H](Oc1cc(-c2cnn(C3CCNCC3)c2)cnc1N)c1c(Cl)ccc(F)c1Cl ; Cc1ccc(S(=O)(=O)OCCOCCOCCOCCOCCOCCOCC(=O)OC(C)(C)C)cc1 ; C[C@@H](Oc1cc(-c2cnn(C3CCN(CCOCCOCCOCCOCCOCCOCC(=O)OC(C)(C)C)CC3)c2)cnc1N)c1c(Cl)ccc(F)c1Cl ; "LiOH" ; C[C@@H](Oc1cc(-c2cnn(C3CCN(CCOCCOCCOCCOCCOCCOCC(=O)OC(C)(C)C)CC3)c2)cnc1N)c1c(Cl)ccc(F)c1Cl ; "(I-3)"]

Step 1. To a solution of (R)-3-(1-(2,6-dichloro-3-fluorophenyl)ethoxy)-5-(1-(piperidin-4-yl)-1H-pyrazol-4-yl)pyridin-2-amine (100 mg, 0.2200 mmol) and DIPEA (58 μL, 0.33 mmol) in DMF (0.29 mL) at room temperature was added tert-butyl 2-[2-[2-[2-[2-[2-[2-(p-tolylsulfonyloxy)ethoxy]ethoxy]ethoxy]ethoxy]ethoxy]ethoxy]acetate (146.79 mg, 0.27 mmol). DMSO (0.29 mL) was added to achieve complete dissolution. The resulting yellow solution was stirred at room temperature for 4 days. LCMS showed that after 4 days the conversion was stopped around 60. The mixture was purified using reverse phase column chromatography. Fractions were combined and concentrated to afford 110 mg of the desired product as a light yellow solid in 59.8% yield.

LCMS Method 5: retention time: 1.143 min, 90.1% purity at 254 nm, [M+2H]/2⁺=829.2.

Step 2. To a solution of tert-butyl (R)-20-(4-(4-(6-amino-5-(1-(2,6-dichloro-3-fluorophenyl)ethoxy)pyridin-3-yl)-1H-pyrazol-1-yl)piperidin-1-yl)-3,6,9,12,15,18-hexaoxaicosanoate (110 mg, 0.1300 mmol) in water (0.74 mL) and THF (1.48 mL) at room temperature was added LiOH monohydrate (27.85 mg, 0.6600 mmol). The reaction was stirred at 40° C. for 2 hours, and LCMS showed complete conversion. The reaction mixture was concentrated under reduced pressure to remove the THF, and the resulting water solution was loaded directly onto a 30 g gold C18 column to purify by reverse phase column chromatography. The fractions were combined and concentrated to afford 43 mg of the desired product as a light yellow oil in 41.9% yield.

LCMS Method 5: retention time: 0.971 min, 100% purity at 254 nm, [M+H]⁺=772.3 [M+2H]/2⁺=387.8.

### Example I-4. Preparation of (R)-23-(4-(4-(6-amino-5-(1-(2,6-dichloro-3-fluorophenyl)ethoxy)pyridin-3-yl)-1H-pyrazol-1-yl)piperidin-1-yl)-3,6,9,12,15,18,21-heptaoxatricosanoic acid (Intermediate I-4)

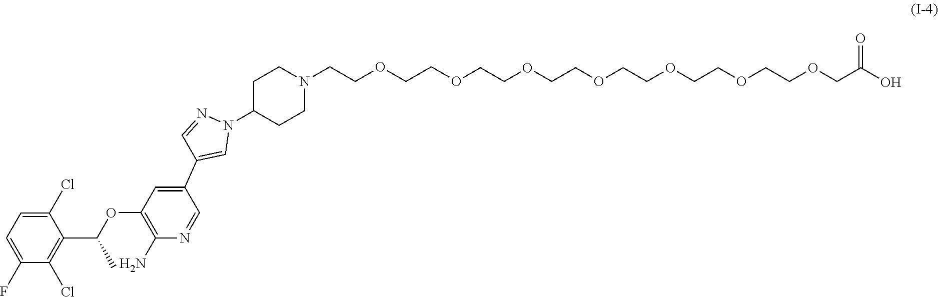 [SMILES: C[C@@H](Oc1cc(-c2cnn(C3CCN(CCOCCOCCOCCOCCOCCOCCOCC(=O)O)CC3)c2)cnc1N)c1c(Cl)ccc(F)c1Cl]

The title compound was prepared using the same procedure as described for I-3 using ethyl 2-[2-[2-[2-[2-[2-[2-[2-(p-tolylsulfonyloxy)ethoxy]ethoxy]ethoxy]ethoxy]ethoxy]ethoxy]ethoxy]acetate.

LCMS Method 5: retention time: 1.040 min, 99.0% purity at 215 nm [M+H]⁺=816.3.

### Example I-5. Preparation of 2-((2-(2,6-dioxopiperidin-3-yl)-1,3-dioxoisoindolin-4-yl)oxy)-N-(2-hydroxyethyl)acetamide (Intermediate I-5)

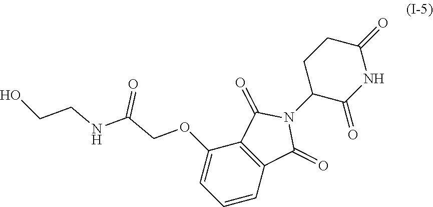 [SMILES: O=C(COc1cccc2c1C(=O)N(C1CCC(=O)NC1=O)C2=O)NCCO]

To a mixture of 2-((2-(2,6-dioxopiperidin-3-yl)-1,3-dioxoisoindolin-4-yl)oxy)acetic acid (1.8 g, 5.42 mmol) and HATU (2.472 g, 6.50 mmol) in DMF (20 ml) was added DIEA (2.84 mL, 16.25 mmol) followed by 2-aminoethan-1-ol (330 mg, 5.42 mmol) at r.t. After the reaction was stirred at r.t. for 1 hour, it was concentrated and purified by normal phase column chromatography (biotage silica, 0-100% EA/hex, then 0-25% MeOH/DCM) to give the desired product.

LC/MS Method 2: MS (ESI) [M+H]⁺ 376.0, rt: 0.59 min.

### Example I-6. Preparation of 2-((2-(2,6-Dioxopiperidin-3-yl)-1,3-dioxoisoindolin-4-yl)oxy)-N-(2-(2-hydroxyethoxy)ethyl)acetamide (Intermediate I-6)

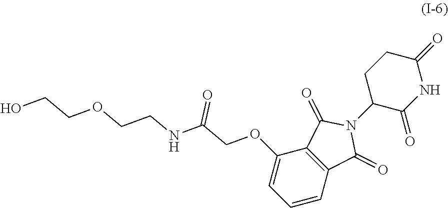 [SMILES: O=C(COc1cccc2c1C(=O)N(C1CCC(=O)NC1=O)C2=O)NCCOCCO]

The title compound was prepared by the same general procedure as described for I-5 using 2-(2-aminoethoxy)ethan-1-ol. LC/MS Method 2: MS (ESI) [M+H]⁺ 420.1, rt: 0.61 min.

### Example I-7. Preparation of 2-((2-(2,6-Dioxopiperidin-3-yl)-1,3-dioxoisoindolin-4-yl)oxy)-N-(2-(2-(2-hydroxyethoxy)ethoxy)ethyl)acetamide (Intermediate I-7)

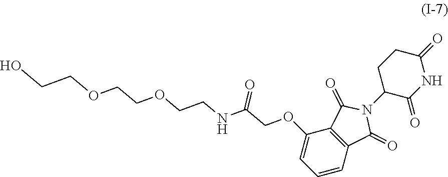 [SMILES: O=C(COc1cccc2c1C(=O)N(C1CCC(=O)NC1=O)C2=O)NCCOCCOCCO]

The title compound was prepared by the same general procedure as described for I-5 using 2-(2-(2-aminoethoxy)ethoxy)ethan-1-ol. LC/MS Method 2: MS (ESI) [M+H]⁺ 464.1, rt: 0.64 min.

### Example I-8. Preparation of 2-((2-(2,6-Dioxopiperidin-3-yl)-1,3-dioxoisoindolin-4-yl)oxy)-N-(2-(2-(2-(2-hydroxyethoxy)ethoxy)ethoxy)ethyl)acetamide (Intermediate I-8)

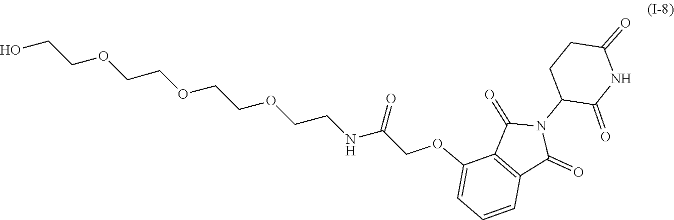 [SMILES: O=C(COc1cccc2c1C(=O)N(C1CCC(=O)NC1=O)C2=O)NCCOCCOCCOCCO]

The title compound was prepared by the same general procedure as described for I-5 using 2-(2-(2-(2-aminoethoxy)ethoxy)ethoxy)ethan-1-ol. LC/MS Method 2: MS (ESI) [M+H]⁺ 508.1, rt: 0.66 min.

### Example I-9. Preparation of 2-((2-(2,6-Dioxopiperidin-3-yl)-1,3-dioxoisoindolin-4-yl)oxy)-N-(14-hydroxy-3,6,9,12-tetraoxatetradecyl)acetamide (Intermediate I-9)

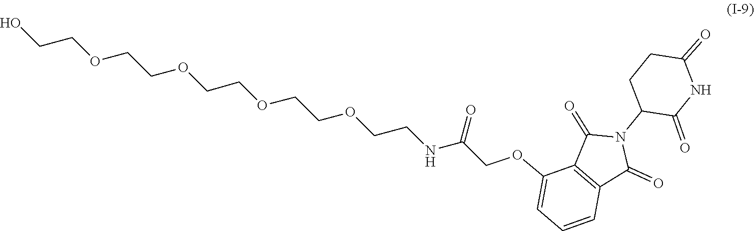 [SMILES: O=C(COc1cccc2c1C(=O)N(C1CCC(=O)NC1=O)C2=O)NCCOCCOCCOCCOCCO]

The title compound was prepared by the same general procedure as described for I-5 using 14-amino-3,6,9,12-tetraoxatetradecan-1-ol. LC/MS Method 2: MS (ESI) [M+H]⁺ 552.2, rt: 0.68 min.

### Example 1-10. Preparation of 2-((2-(2,6-Dioxopiperidin-3-yl)-1,3-dioxoisoindolin-5-yl)oxy)-N-(2-hydroxyethyl)acetamide (Intermediate I-10)

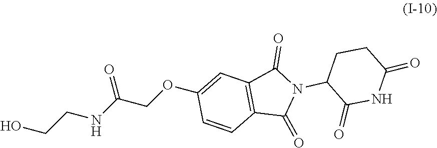 [SMILES: O=C(COc1ccc2c(c1)C(=O)N(C1CCC(=O)NC1=O)C2=O)NCCO]

The title compound was prepared by the same general procedure as described for I-5 using 2-aminoethan-1-ol. LC/MS Method 2: MS (ESI) [M+H]⁺ 376.0, rt: 0.56 min.

### Example I-11. Preparation of 2-((2-(2,6-Dioxopiperidin-3-yl)-1,3-dioxoisoindolin-5-yl)oxy)-N-(2-(2-hydroxyethoxy)ethyl)acetamide (Intermediate I-11)

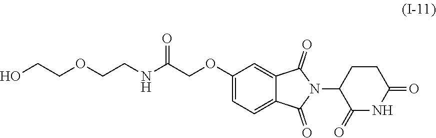 [SMILES: O=C(COc1ccc2c(c1)C(=O)N(C1CCC(=O)NC1=O)C2=O)NCCOCCO]

The title compound was prepared by the same general procedure as described for I-5 using 2-(2-aminoethoxy)ethan-1-ol. LC/MS Method 2: MS (ESI) [M+H]⁺ 420.1, rt: 0.61 min.

### Example I-12. Preparation of 2-((2-(2,6-Dioxopiperidin-3-yl)-1,3-dioxoisoindolin-5-yl)oxy)-N-(2-(2-(2-hydroxyethoxy)ethoxy)ethyl)acetamide (Intermediate I-12)

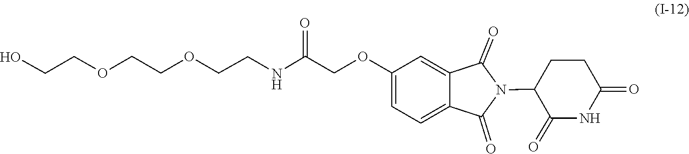 [SMILES: O=C(COc1ccc2c(c1)C(=O)N(C1CCC(=O)NC1=O)C2=O)NCCOCCOCCO]

The title compound was prepared by the same general procedure as described for I-5 using 2-(2-(2-aminoethoxy)ethoxy)ethan-1-ol. LC/MS Method 2: MS (ESI) [M+H]⁺ 464.1, rt: 0.63 min.

### Example I-13. Preparation of 2-((2-(2,6-Dioxopiperidin-3-yl)-1,3-dioxoisoindolin-5-yl)oxy)-N-(2-(2-(2-(2-hydroxyethoxy)ethoxy)ethoxy)ethyl)acetamide (Intermediate I-13)

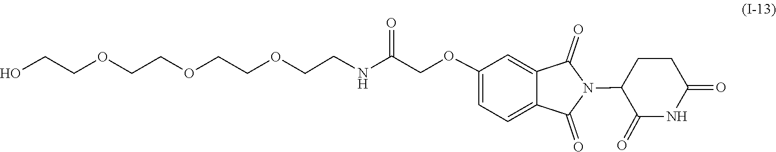 [SMILES: O=C(COc1ccc2c(c1)C(=O)N(C1CCC(=O)NC1=O)C2=O)NCCOCCOCCOCCO]

The title compound was prepared by the same general procedure as described for I-5 using 2-(2-(2-(2-aminoethoxy)ethoxy)ethoxy)ethan-1-ol. LC/MS Method 2: MS (ESI) [M+H]⁺ 508.1, rt: 0.65 min.

### Example I-14. Preparation of 2-((2-(2,6-dioxopiperidin-3-yl)-1,3-dioxoisoindolin-5-yl)oxy)-N-(14-hydroxy-3,6,9,12-tetraoxatetradecyl)acetamide (Intermediate I-14)

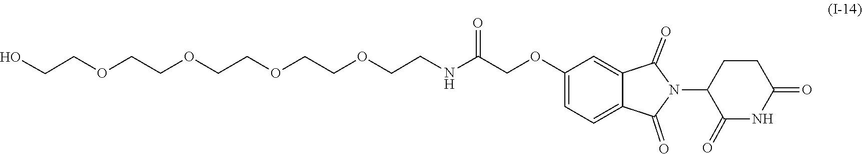 [SMILES: O=C(COc1ccc2c(c1)C(=O)N(C1CCC(=O)NC1=O)C2=O)NCCOCCOCCOCCOCCO]

The title compound was prepared by the same general procedure as described for I-5 using 2-(2-(2-(2-aminoethoxy)ethoxy)ethoxy)ethan-1-ol. LC/MS Method 2: MS (ESI) [M+H]⁺ 552.2, rt: 0.67 min.

### Example I-15. Preparation of (R)-5-(1-(1-(3-azaspiro[5.5]undecan-9-yl)piperidin-4-yl)-1H-pyrazol-4-yl)-3-(1-(2,6-dichloro-3-fluorophenyl)ethoxy)pyridin-2-amine (Intermediate I-15)

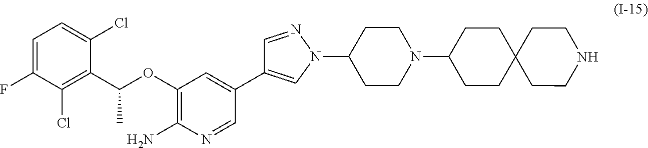 [SMILES: C[C@@H](Oc1cc(-c2cnn(C3CCN(C4CCC5(CCNCC5)CC4)CC3)c2)cnc1N)c1c(Cl)ccc(F)c1Cl]

(R)-3-(1-(2,6-Dichloro-3-fluorophenyl)ethoxy)-5-(1-(piperidin-4-yl)-1H-pyrazol-4-yl)pyridin-2-amine (300 mg, 0.67 mmol), tert-butyl 9-oxo-3-azaspiro[5.5]undecane-3-carboxylate (178 mg, 0.67 mmol), and sodium triacetoxyborohydride (211 mg, 1.0 mmol) were added to a 20 mL vial equipped with stir bar. The vial was then sealed with a cap and purged with nitrogen. The solids were then suspended in DCE (2.7 mL). The cloudy suspension was then treated with acetic acid (60 μL, 1.0 mmol), upon which the solution turned translucent. The reaction was then allowed to stir at 44° C. overnight. LCMS analysis of the reaction mixture showed conversion to the desired product. The residue was then purified by reverse phase column chromatography (15% to 100% MeCN in water, 0.1% TFA). The collected fractions were concentrated and immediately taken up in 1:1 DCM/TFA and left to stir for 2 h. LCMS analysis of the reaction mixture revealed full deprotection, affording the TFA salt of the product, (R)-5-(1-(1-(3-azaspiro[5.5]undecan-9-yl)piperidin-4-yl)-1H-pyrazol-4-yl)-3-(1-(2,6-dichloro-3-fluorophenyl)ethoxy)pyridin-2-amine (390 mg, 0.54 mmol, 81% yield), as an off-white salt after lyophilization. LC/MS Method 2: MS (ESI) [M+2H]/2⁺ 301.5, rt: 0.82 min.

### Example I-16. 2-(2,6-dioxopiperidin-3-yl)-3-oxoisoindoline-5-carboxylic acid (Intermediate I-16)

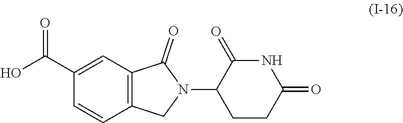 [SMILES: O=C1CCC(N2Cc3ccc(C(=O)O)cc3C2=O)C(=O)N1]

The title compound was commercially available from Enamine.

### Example I-17. 2-(2,6-dioxopiperidin-3-yl)-1-oxoisoindoline-5-carboxylic acid (Intermediate I-17)

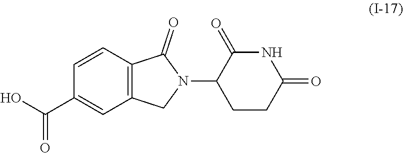 [SMILES: O=C1CCC(N2Cc3cc(C(=O)O)ccc3C2=O)C(=O)N1]

The title compound was commercially available from Enamine.

### Example I-18. Preparation of 2-(2,6-dioxopiperidin-3-yl)-1-oxoisoindoline-4-carboxylic acid (Intermediate I-18)

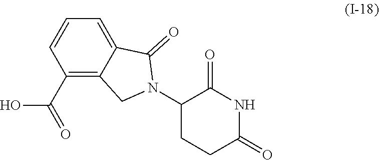 [SMILES: O=C1CCC(N2Cc3c(C(=O)O)cccc3C2=O)C(=O)N1]

The title compound was commercially available from Enamine.

### Example I-19. Preparation of 2-(2,6-dioxopiperidin-3-yl)-1,3-dioxoisoindoline-5-carboxylic acid (Intermediate I-19)

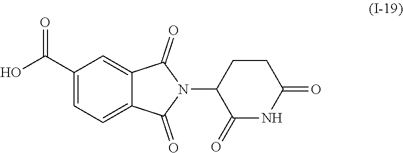 [SMILES: O=C1CCC(N2C(=O)c3ccc(C(=O)O)cc3C2=O)C(=O)N1]

The title compound was commercially available from Enamine.

### Example S1. Preparation of (2S,4R)-1-((S)-2-(4-(4-(4-(6-amino-5-((R)-1-(2,6-dichloro-3-fluorophenyl)ethoxy)pyridin-3-yl)-1H-pyrazol-1-yl)piperidin-1-yl)butanamido)-3,3-dimethylbutanoyl)-4-hydroxy-N-(4-(4-methylthiazol-5-yl)benzyl)pyrrolidine-2-carboxamide (Compound 1)

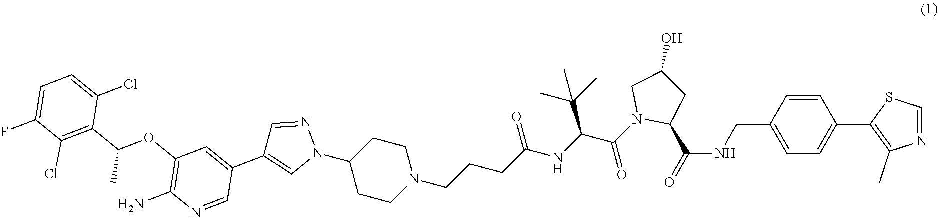 [SMILES: Cc1ncsc1-c1ccc(CNC(=O)[C@@H]2C[C@@H](O)CN2C(=O)[C@@H](NC(=O)CCCN2CCC(n3cc(-c4cnc(N)c(O[C@H](C)c5c(Cl)ccc(F)c5Cl)c4)cn3)CC2)C(C)(C)C)cc1]

(R)-4-(4-(4-(6-amino-5-(1-(2,6-dichloro-3-fluorophenyl)ethoxy)pyridin-3-yl)-1H-pyrazol-1-yl)piperidin-1-yl)butanoic acid (50 mg, 0.09 mmol), (2S,4R)-1-((S)-2-amino-3,3-dimethylbutanoyl)-4-hydroxy-N-(4-(4-methylthiazol-5-yl)benzyl)pyrrolidine-2-carboxamide (52.12 mg, 0.11 mmol), and DIPEA (0.06 mL, 0.37 mmol) were solubilized in DMF (0.93 mL). HATU (43 mg, 0.11 mmol) was added at room temperature for 3 hours. LCMS indicated full conversion. The reaction mixture was purified using reverse phase chromatography (5% to 100% MeCN in water, 0.1% formic acid), washed with MeOH and collected with ammonium solution (3N MeOH). The solution was concentrated under reduced pressure and lyophilized to afford the desired product as a white solid in 16.7% yield (15 mg).

LC/MS Method 3: 98.7% purity at 215 nm, [M+H]⁺=948.2, 950.2 [M+H]/2⁺=474.6, 475.4.

¹H NMR (400 MHz, DMSO-d₆) δ ppm 8.97 (s, 1H), 8.55 (t, J=5.6 Hz, 1H), 7.93 (s, 1H), 7.86 (d, J=9.1 Hz, 1H), 7.74 (d, J=1.5 Hz, 1H), 7.49-7.62 (m, 2H), 7.29-7.47 (m, 5H), 6.89 (s, 1H), 6.08 (q, J=6.7 Hz, 1H), 5.63 (s, 2H), 5.13 (d, J=3.5 Hz, 1H), 5.13 (d, J=3.5 Hz, 1H), 4.55 (d, J=9.3 Hz, 1H), 4.38-4.49 (m, 2H), 4.35 (br. s, 1H), 4.21 (dd, J=15.8, 5.7 Hz, 1H), 4.08 (br. s, 1H), 3.60-3.73 (m, 2H), 2.92 (d, J=10.1 Hz, 2H), 2.44 (s, 3H), 2.12-2.36 (m, 4H), 1.85-2.09 (m, 8H), 1.80 (d, J=6.6 Hz, 3H), 1.57-1.74 (m, 2H), 0.94 (s, 9H).

### Example S2. Preparation of (2S,4R)-1-((S)-2-(2-(2-(2-(4-(4-(6-amino-5-((R)-1-(2,6-dichloro-3-fluorophenyl)ethoxy)pyridin-3-yl)-1H-pyrazol-1-yl)piperidin-1-yl)ethoxy)ethoxy)acetamido)-3,3-dimethylbutanoyl)-4-hydroxy-N-(4-(4-methylthiazol-5-yl)benzyl)pyrrolidine-2-carboxamide (Compound 2)

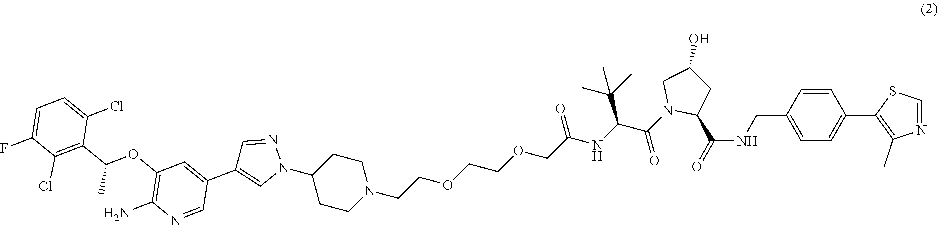 [SMILES: Cc1ncsc1-c1ccc(CNC(=O)[C@@H]2C[C@@H](O)CN2C(=O)[C@@H](NC(=O)COCCOCCN2CCC(n3cc(-c4cnc(N)c(O[C@H](C)c5c(Cl)ccc(F)c5Cl)c4)cn3)CC2)C(C)(C)C)cc1]

To a solution of Crizotinib (R)-3-(1-(2,6-dichloro-3-fluorophenyl)ethoxy)-5-(1-(piperidin-4-yl)-1H-pyrazol-4-yl)pyridin-2-amine (40 mg, 0.09 mmol) and 2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl 4-methylbenzenesulfonate (77.8 mg, 0.11 mmol) in DMF (400 μL)/DMSO (100 μL) at room temperature was added DIPEA (50 μL, 0.29 mmol). The reaction mixture was stirred at room temperature for 48 hours. The LCMS show near completion. The reaction mixture was purified using reverse phase column chromatography. Fractions were combined and concentrated to afford 43.6 mg of the desired product as a white solid (49% yield).

LC/MS Method 3: [M+2H]/2⁺=504.6, [M+3H]/3⁺=336.8, rt=2.359 min.

¹H NMR (400 MHz, DMSO-d₆) δ ppm 8.96-8.99 (m, 1H), 8.59 (t, J=6.2 Hz, 1H), 7.92-7.95 (m, 1H), 7.75 (d, J=2.0 Hz, 1H), 7.56 (dd, J=9.1, 5.1 Hz, 1H), 7.51 (s, 1H), 7.41-7.46 (m, 2H), 6.88-6.92 (m, 1H), 6.08 (q, J=6.6 Hz, 1H), 5.63 (s, 2H), 5.15 (d, J=3.5 Hz, 1H), 4.57 (d, J=9.6 Hz, 1H), 4.32-4.47 (m, 4H), 4.20-4.28 (m, 1H), 4.00-4.11 (m, 2H), 3.98 (s, 2H), 3.52-3.70 (m, 9H), 3.17 (d, J=5.3 Hz, 1H), 2.89-2.99 (m, 3H), 2.43 (s, 3H), 2.04-2.14 (m, 3H), 1.85-1.97 (m, 6H), 1.79 (d, J=6.6 Hz, 3H), 0.95 (s, 9H).

### Example S3. Preparation of (2S,4R)-1-((S)-14-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (Compound 3)

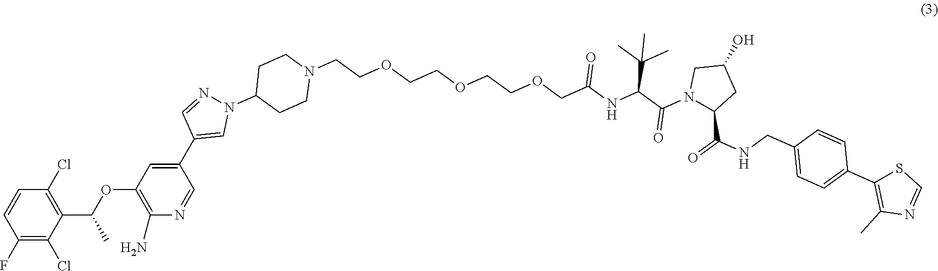 [SMILES: Cc1ncsc1-c1ccc(CNC(=O)[C@@H]2C[C@@H](O)CN2C(=O)[C@@H](NC(=O)COCCOCCOCCN2CCC(n3cc(-c4cnc(N)c(O[C@H](C)c5c(Cl)ccc(F)c5Cl)c4)cn3)CC2)C(C)(C)C)cc1]

Step 1: Ethyl (R)-2-(2-(2-(2-(4-(4-(6-amino-5-(1-(2,6-dichloro-3-fluorophenyl)ethoxy)pyridin-3-yl)-1H-pyrazol-1-yl)piperidin-1-yl)ethoxy)ethoxy)ethoxy)acetate. To a solution of ethyl 2-[2-[2-[2-(p-tolylsulfonyloxy)ethoxy]ethoxy]ethoxy]acetate (208 mg, 0.53 mmol) and (R)-3-(1-(2,6-dichloro-3-fluorophenyl)ethoxy)-5-(1-(piperidin-4-yl)-1H-pyrazol-4-yl)pyridin-2-amine (200 mg, 0.44 mmol) in DMF (580 μL) at room temperature was added DIPEA (117 μL, 0.67 mmol). Additional DMSO (580 μL) was then added to achieve complete solubilization. The resulting yellow solution was stirred overnight at room temperature. LCMS analysis revealed ~75% conversion to the product. The mixture was purified using reverse phase column chromatography (5% to 100% MeCN in water, 0.1% formic acid), affording the product, ethyl (R)-2-(2-(2-(2-(4-(4-(6-amino-5-(1-(2,6-dichloro-3-fluorophenyl)ethoxy)pyridin-3-yl)-1H-pyrazol-1-yl)piperidin-1-yl)ethoxy)ethoxy)ethoxy)acetate (225 mg, 0.34 mmol, 76% yield), as a light yellow oil after concentration. LC/MS Method 1: MS (ESI) [M+2H]/2⁺ 335.8, rt: 0.980 min.

Step 2: (R)-2-(2-(2-(2-(4-(4-(6-Amino-5-(1-(2,6-dichloro-3-fluorophenyl)ethoxy)pyridin-3-yl)-1H-pyrazol-1-yl)piperidin-1-yl)ethoxy)ethoxy)ethoxy)acetic acid. To a solution of ethyl (R)-2-(2-(2-(2-(4-(4-(6-amino-5-(1-(2,6-dichloro-3-fluorophenyl)ethoxy)pyridin-3-yl)-1H-pyrazol-1-yl)piperidin-1-yl)ethoxy)ethoxy)ethoxy)acetate (225 mg, 0.34 mmol) in water (1.9 mL) was added LiOH monohydrate (70.6 mg, 1.68 mmol). After 2 h, LCMS showed the reaction went to completion. The reaction mixture was concentrated and purified by reverse phase column chromatography (5% to 100% MeCN in water), affording the product, (R)-2-(2-(2-(2-(4-(4-(6-Amino-5-(1-(2,6-dichloro-3-fluorophenyl)ethoxy)pyridin-3-yl)-1H-pyrazol-1-yl)piperidin-1-yl)ethoxy)ethoxy)ethoxy)acetic acid (quantitative), as a light yellow oil after concentration. LC/MS Method 1: MS (ESI) [M+2H]/2⁺ 321.6, rt: 0.937 min.

Step 3: (2S,4R)-1-((S)-14-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide. To a solution of (R)-2-(2-(2-(2-(4-(4-(6-Amino-5-(1-(2,6-dichloro-3-fluorophenyl)ethoxy)pyridin-3-yl)-1H-pyrazol-1-yl)piperidin-1-yl)ethoxy)ethoxy)ethoxy)acetic acid (200 mg, 0.31 mmol) in DMF (3.2 mL) at room temperature were added DIPEA (193 μL, 1.11 mmol) and (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (160 mg, 0.34 mmol). Then, HATU (120 mg, 0.32 mmol) was added in one portion and the resulting yellow solution was stirred at room temperature. After 2 h, LCMS analysis revealed that the reaction went to completion. The reaction mixture was purified by reverse phase column chromatography (5% to 100% MeCN in water, 0.1% formic acid), and the fractions were combined and concentrated. The resulting oil that was obtained was re-purified by reverse phase column chromatography (5% MeCN in pH 10 water), affording the product, (2S,4R)-1-((S)-14-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (50 mg, 0.047 mmol, 15% yield), as a white solid after lyophilization.

¹H NMR (400 MHz, DMSO-d₆) δ ppm 8.98 (s, 1H), 8.61 (t, J=5.9 Hz, 1H), 7.94 (s, 1H), 7.74 (s, 1H), 7.54-7.60 (m, 1H), 7.52 (s, 1H), 7.37-7.46 (m, 6H), 6.88 (s, 1H), 6.08 (q, J=6.7 Hz, 1H), 5.65 (s, 2H), 5.16 (d, J 3.3 Hz, 1H), 4.56 (d, J 9.9 Hz, 1H), 4.32-4.48 (m, 3H), 4.20-4.29 (m, 1H), 4.01-4.13 (m, 1H), 3.94-4.00 (m, 2H), 3.42-3.73 (m, 14H), 2.90-3.04 (m, 2H), 2.44 (s, 3H), 2.01-2.24 (m, 2H), 1.84-1.99 (m, 4H), 1.79 (d, J=6.6 Hz, 3H), 0.94 (s, 9H).

LC/MS Method 3: MS (ESI) [M+2H]/2⁺ 526.7, rt: 2.338 min.

### Example S4. Preparation of (2S,4R)-1-((S)-17-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12,15-tetraoxa-3-azaheptadecanoyl)-4-hydroxy-N-(4-(4-methylthiazol-5-yl)benzyl)pyrrolidine-2-carboxamide (Compound 4)

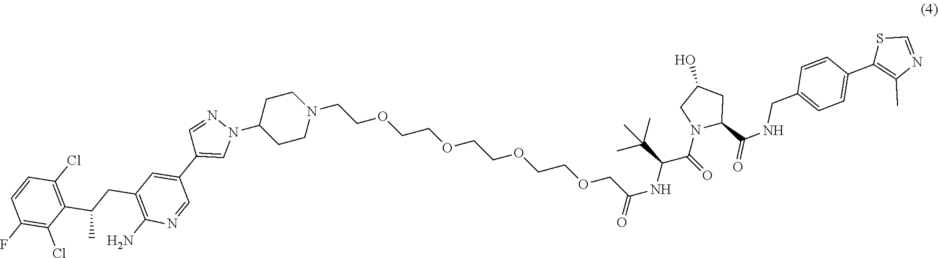 [SMILES: Cc1ncsc1-c1ccc(CNC(=O)[C@@H]2C[C@@H](O)CN2C(=O)[C@@H](NC(=O)COCCOCCOCCOCCN2CCC(n3cc(-c4cnc(N)c(C[C@H](C)c5c(Cl)ccc(F)c5Cl)c4)cn3)CC2)C(C)(C)C)cc1]

Step 1: tert-butyl (R)-14-(4-(4-(6-amino-5-(1-(2,6-dichloro-3-fluorophenyl)ethoxy)pyridin-3-yl)-1H-pyrazol-1-yl)piperidin-1-yl)-3,6,9,12-tetraoxatetradecanoate. To a solution of tert-butyl 14-(tosyloxy)-3,6,9,12-tetraoxatetradecanoate (247 mg, 0.53 mmol) and (R)-3-(1-(2,6-dichloro-3-fluorophenyl)ethoxy)-5-(1-(piperidin-4-yl)-1H-pyrazol-4-yl)pyridin-2-amine (200 mg, 0.44 mmol) in DMF (580 μL) at room temperature was added DIPEA (117 μL, 0.67 mmol). Then, additional DMSO (580 μL) was added to fully solubilize the materials. The resulting yellow solution was stirred at room temperature overnight. LCMS analysis revealed approximately 50% conversion to the desired product. The reaction was stirred for a further 3 h, at which point the reaction mixture was directly purified by reverse phase column chromatography (5% to 100% MeOH in water, 0.1% formic acid). The fractions were combined and concentrated, affording the product, tert-butyl (R)-14-(4-(4-(6-amino-5-(1-(2,6-dichloro-3-fluorophenyl)ethoxy)pyridin-3-yl)-1H-pyrazol-1-yl)piperidin-1-yl)-3,6,9,12-tetraoxatetradecanoate (127 mg, 0.17 mmol, 38% yield), as a light yellow oil after concentration. LC/MS Method 1: MS (ESI) [M+2H]/2⁺ 371.6, rt: 1.038 min.

Step 2: (R)-14-(4-(4-(6-amino-5-(1-(2,6-dichloro-3-fluorophenyl)ethoxy)pyridin-3-yl)-1H-pyrazol-1-yl)piperidin-1-yl)-3,6,9,12-tetraoxatetradecanoic acid. To a solution of tert-butyl (R)-14-(4-(4-(6-amino-5-(1-(2,6-dichloro-3-fluorophenyl)ethoxy)pyridin-3-yl)-1H-pyrazol-1-yl)piperidin-1-yl)-3,6,9,12-tetraoxatetradecanoate (127 mg, 0.17 mmol) in DCM (725 μL) was added TFA (870 μL, 11 mmol) dropwise. The resulting solution was stirred at room temperature for 2 h, after which time LCMS analysis revealed that the reaction went to completion. The solvent was removed under reduced pressure, and residual TFA was removed by co-evaporation with toluene (2×5 mL). The residue was purified by reverse phase column chromatography (5% to 100% MeCN in pH 10 water), affording the product, (R)-14-(4-(4-(6-amino-5-(1-(2,6-dichloro-3-fluorophenyl)ethoxy)pyridin-3-yl)-1H-pyrazol-1-yl)piperidin-1-yl)-3,6,9,12-tetraoxatetradecanoic acid (70 mg, 0.10 mmol, 59% yield), as a light yellow oil after concentration. LC/MS Method 1: MS (ESI) [M+2H]/2⁺ 342.8, rt: 0.955 min.

Step 3: (2S,4R)-1-((S)-17-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12,15-tetraoxa-3-azaheptadecanoyl)-4-hydroxy-N-(4-(4-methylthiazol-5-yl)benzyl)pyrrolidine-2-carboxamide. To a solution of (R)-14-(4-(4-(6-amino-5-(1-(2,6-dichloro-3-fluorophenyl)ethoxy)pyridin-3-yl)-1H-pyrazol-1-yl)piperidin-1-yl)-3,6,9,12-tetraoxatetradecanoic acid (70 mg, 0.10 mmol) in DMF (1.05 mL) at room temperature were added DIPEA (63 μL, 0.36 mmol) and (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (52 mg, 0.11 mmol). Then, HATU (39 mg, 0.10 mmol) was added in one portion, and the resulting yellow solution was stirred at room temperature. After 2 h, LCMS analysis revealed that the reaction went to completion. The reaction mixture was purified by reverse phase column chromatography (5% to 100% MeCN in water, 0.1% formic acid), the resulting fractions were combined, concentrated and re-purified by reverse phase column chromatography (5% to 100% MeCN in pH 10 water). The fractions were combined and concentrated to give the product with ~85% purity. The material was then further re-purified by reverse phase column chromatography (5% to 100% MeCN in water, 0.1% formic acid) and neutralized through the use of a catch and release purification on an SPE cartridge (tosic acid), affording the product, (2S,4R)-1-((S)-17-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12,15-tetraoxa-3-azaheptadecanoyl)-4-hydroxy-N-(4-(4-methylthiazol-5-yl)benzyl)pyrrolidine-2-carboxamide (8.2 mg, 0.007 mmol, 7% yield), as an off-white solid after lyophilization.

¹H NMR (400 MHz, DMSO-d₆) δ ppm 8.98 (s, 1H), 8.60 (br t, J=5.2 Hz, 1H), 7.94 (s, 1H), 7.74 (s, 1H), 7.54-7.60 (m, 1H), 7.52 (s, 1H), 7.37-7.46 (m, 6H), 6.89 (s, 1H), 6.08 (q, J=6.6 Hz, 1H), 5.65 (s, 2H), 5.16 (br s, 1H), 4.56 (br d, J=9.6 Hz, 1H), 4.31-4.48 (m, 3H), 4.17-4.31 (m, 1H), 4.03-4.16 (m, 1H), 3.94-3.99 (m, 2H), 3.42-3.69 (m, 18H), 2.95-3.10 (m, 2H), 2.55-2.64 (m, 2H) 2.43 (s, 3H), 2.14-2.29 (m, 2H), 1.85-2.11 (m, 4H), 1.79 (br d, J=6.6 Hz, 3H), 0.93 (s, 9H).

LC/MS Method 3: MS (ESI) [M+2H]/2⁺ 548.8, rt: 2.406 min.

### Example S5. Preparation of (2S,4R)-1-((S)-20-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12,15,18-pentaoxa-3-azaicosanoyl)-4-hydroxy-N-(4-(4-methylthiazol-5-yl)benzyl)pyrrolidine-2-carboxamide (Compound 5)

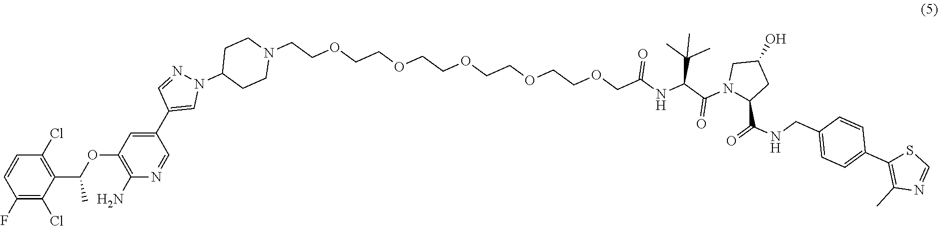 [SMILES: Cc1ncsc1-c1ccc(CNC(=O)[C@@H]2C[C@@H](O)CN2C(=O)[C@@H](NC(=O)COCCOCCOCCOCCOCCN2CCC(n3cc(-c4cnc(N)c(O[C@H](C)c5c(Cl)ccc(F)c5Cl)c4)cn3)CC2)C(C)(C)C)cc1]

Step 1: Ethyl (R)-17-(4-(4-(6-amino-5-(1-(2,6-dichloro-3-fluorophenyl)ethoxy)pyridin-3-yl)-1H-pyrazol-1-yl)piperidin-1-yl)-3,6,9,12,15-pentaoxaheptadecanoate. To a solution of ethyl 17-bromo-3,6,9,12,15-pentaoxaheptadecanoate (103 mg, 0.27 mmol) and (R)-3-(1-(2,6-dichloro-3-fluorophenyl)ethoxy)-5-(1-(piperidin-4-yl)-1H-pyrazol-4-yl)pyridin-2-amine (100 mg, 0.22 mmol) in DMF (800 L) at room temperature was added DIPEA (58 uL, 0.33 mmol). Then, DMSO (290 μL) was added to achieve complete solubilization. The resulting yellow solution was stirred at room temperature 2 days, at which point LCMS analysis revealed the reaction went to completion. The mixture was purified by reverse phase column chromatography (5% to 100% MeCN in water, 0.1% formic acid), affording the product, ethyl (R)-17-(4-(4-(6-amino-5-(1-(2,6-dichloro-3-fluorophenyl)ethoxy)pyridin-3-yl)-1H-pyrazol-1-yl)piperidin-1-yl)-3,6,9,12,15-pentaoxaheptadecanoate (125 mg, 0.16 mmol, 74% yield), as a light yellow solid. LC/MS Method 1: MS (ESI) [M+2H]/2⁺ 379.6, rt: 1.001 min.

Step 2: (R)-17-(4-(4-(6-amino-5-(1-(2,6-dichloro-3-fluorophenyl)ethoxy)pyridin-3-yl)-1H-pyrazol-1-yl)piperidin-1-yl)-3,6,9,12,15-pentaoxaheptadecanoic acid. To a solution of LiOH monohydrate (35 mg, 0.83 mmol) in THE (1.5 mL) and water (1 mL) was added ethyl (R)-17-(4-(4-(6-amino-5-(1-(2,6-dichloro-3-fluorophenyl)ethoxy)pyridin-3-yl)-1H-pyrazol-1-yl)piperidin-1-yl)-3,6,9,12,15-pentaoxaheptadecanoate (125 mg, 0.17 mmol). After 2 h, LCMS showed the reaction went to completion. The reaction mixture was concentrated and then purified by reverse phase column chromatography (5% to 100% MeCN in water), affording the product (R)-17-(4-(4-(6-amino-5-(1-(2,6-dichloro-3-fluorophenyl)ethoxy)pyridin-3-yl)-1H-pyrazol-1-yl)piperidin-1-yl)-3,6,9,12,15-pentaoxaheptadecanoic acid (82 mg, 0.11 mmol, 68% yield), as a light yellow oil. LC/MS Method 1: MS (ESI) [M+2H]/2⁺ 364.8, rt: 0.977 min.

Step 3: (2S,4R)-1-((S)-20-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12,15,18-pentaoxa-3-azaicosanoyl)-4-hydroxy-N-(4-(4-methylthiazol-5-yl)benzyl)pyrrolidine-2-carboxamide. To a solution of (R)-17-(4-(4-(6-amino-5-(1-(2,6-dichloro-3-fluorophenyl)ethoxy)pyridin-3-yl)-1H-pyrazol-1-yl)piperidin-1-yl)-3,6,9,12,15-pentaoxaheptadecanoic acid (78 mg, 0.11 mmol) in DMF (1.1 mL) at room temperature were added DIPEA (66 μL, 0.38 mmol) and (2S,4R)-1-((S)-2-amino-3,3-dimethylbutanoyl)-4-hydroxy-N-(4-(4-methylthiazol-5-yl)benzyl)pyrrolidine-2-carboxamide hydrochloride (55 mg, 0.12 mmol). Then, HATU (41 mg, 0.11 mmol) was added in one portion, and the resulting yellow solution was stirred at room temperature. After 2 h, LCMS showed that the reaction went to completion. The reaction mixture was purified by reverse phase column chromatography (5% to 100% MeCN in water, 0.1% formic acid), affording mixed fractions which were concentrated and repurified by reverse phase column chromatography (5% to 100% MeCN in pH 10 water), affording the product, (2S,4R)-1-((S)-20-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12,15,18-pentaoxa-3-azaicosanoyl)-4-hydroxy-N-(4-(4-methylthiazol-5-yl)benzyl)pyrrolidine-2-carboxamide (85 mg, 0.074 mmol, 69% yield) as a white solid after lyophilization.

¹H NMR (400 MHz, DMSO-d₆) δ ppm 8.98 (s, 1H), 8.60 (t, J=6.2 Hz, 1H), 7.95 (s, 1H), 7.75 (d, J=1.5 Hz, 1H), 7.54-7.61 (m, 1H), 7.52 (s, 1H), 7.37-7.46 (m, 6H), 6.89 (s, 1H), 6.08 (q, J=6.8 Hz, 1H), 5.64 (s, 2H), 5.15 (d, J=3.5 Hz, 1H), 4.57 (d, J=9.3 Hz, 1H), 4.44 (m, 3H), 4.21-4.30 (m, 1H), 4.02-4.12 (m, 1H), 3.94-3.99 (m, 2H), 3.45-3.71 (m, 22H), 2.91-3.00 (m, 2H), 2.44 (s, 3H), 2.02-2.18 (m, 2H), 1.86-2.00 (m, 4H), 1.78-1.86 (m, 3H), 0.90-0.99 (m, 9H). Two aliphatic protons undetected.

LC/MS Method 3: MS (ESI) [M+2H]/2⁺ 570.8, rt: 2.439 min.

### Example S6. Preparation of (2S,4R)-1-((S)-23-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12,15,18,21-hexaoxa-3-azatricosanoyl)-4-hydroxy-N-(4-(4-methylthiazol-5-yl)benzyl)pyrrolidine-2-carboxamide (Compound 6)

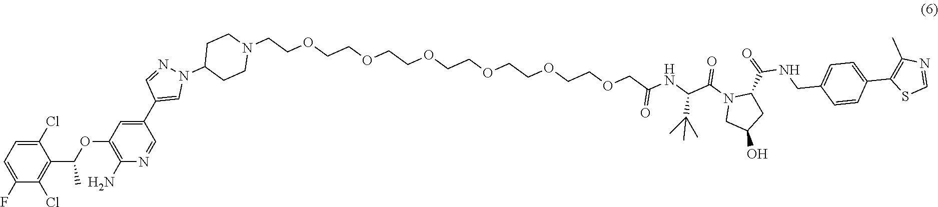 [SMILES: Cc1ncsc1-c1ccc(CNC(=O)[C@@H]2C[C@@H](O)CN2C(=O)[C@@H](NC(=O)COCCOCCOCCOCCOCCOCCN2CCC(n3cc(-c4cnc(N)c(O[C@H](C)c5c(Cl)ccc(F)c5Cl)c4)cn3)CC2)C(C)(C)C)cc1]

To a solution of HATU (23.28 mg, 0.0600 mmol) in DMF (0.5737 mL) at room temperature were added DIPEA (33.93 μL, 0.1900 mmol) and (R)-3-(1-(2,6-dichloro-3-fluorophenyl)ethoxy)-5-(1-(piperidin-4-yl)-1H-pyrazol-4-yl)pyridin-2-amine; hydrochloride (28.59 mg, 0.06 mmol). Then, (R)-20-(4-(4-(6-amino-5-(1-(2,6-dichloro-3-fluorophenyl)ethoxy)pyridin-3-yl)-1H-pyrazol-1-yl)piperidin-1-yl)-3,6,9,12,15,18-hexaoxaicosanoic acid (43 mg, 0.06 mmol) was added in one portion and the resulting yellow solution was stirred at room temperature. After 2 h, LCMS showed complete conversion. The reaction mixture was purified using reverse phase column chromatography (5% to 100% MeCN in water, 0.1% formic acid). The fractions were combined and concentrated, and it was purified by preparative HPLC. A catch and release purification on an SPE cartridge (tosic acid) was run on the product to afford 6 mg the desired product as a white solid in 9.0% yield.

LC/MS Method 3: retention time: 2.480 min, 98.9% purity at 215 nm, [M+2H]/2⁺=592.7 593.6.

¹H NMR (400 MHz, DMSO-d₆) δ ppm 8.98 (s, 1H), 8.56-8.62 (m, 1H), 7.92-7.97 (m, 1H), 7.74 (br d, J=1.3 Hz, 1H), 7.54-7.59 (m, 1H), 7.52 (s, 1H), 7.38-7.47 (m, 6H), 6.89 (s, 1H), 6.08 (br d, J=6.3 Hz, 1H), 5.64 (s, 2H), 5.14 (br d, J=3.5 Hz, 1H), 4.56 (br d, J=9.3 Hz, 1H), 4.31-4.48 (m, 4H), 4.18-4.30 (m, 1H), 4.03-4.14 (m, 1H), 3.96 (s, 2H), 3.49 (br d, J=11.1 Hz, 25H), 2.88-3.05 (m, 2H), 2.42-2.45 (m, 3H), 2.10-2.20 (m, 2H), 2.02-2.09 (m, 1H), 1.85-2.01 (m, 5H), 1.77-1.83 (m, 3H), 0.90-0.99 (m, 9H).

### Example S7. Preparation of (2S,4R)-1-((S)-26-(4-(4-(6-amino-5-((R)-1-(2,6-dichloro-3-fluorophenyl)ethoxy)pyridin-3-yl)-1H-pyrazol-1-yl)piperidin-1-yl)-2-(tert-butyl)-4-oxo-6,9,12,15,18,21,24-heptaoxa-3-azahexacosanoyl)-4-hydroxy-N-(4-(4-methylthiazol-5-yl)benzyl)pyrrolidine-2-carboxamide (Compound 7)

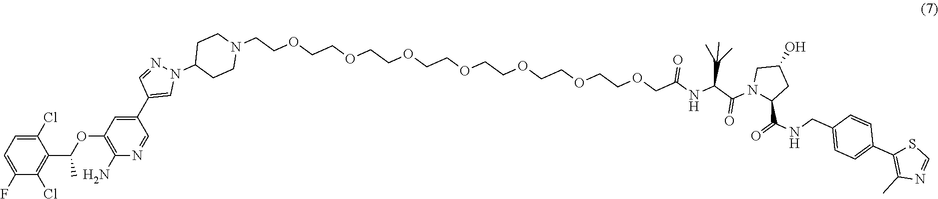 [SMILES: Cc1ncsc1-c1ccc(CNC(=O)[C@@H]2C[C@@H](O)CN2C(=O)[C@@H](NC(=O)COCCOCCOCCOCCOCCOCCOCCN2CCC(n3cc(-c4cnc(N)c(O[C@H](C)c5c(Cl)ccc(F)c5Cl)c4)cn3)CC2)C(C)(C)C)cc1]

To a solution of DIPEA (211.25 μL, 1.21 mmol), HATU (131.68 mg, 0.3500 mmol), and (2S,4R)-1-((S)-2-amino-3,3-dimethylbutanoyl)-4-hydroxy-N-(4-(4-methylthiazol-5-yl)benzyl)pyrrolidine-2-carboxamide (22 mg, 0.05 mmol) in DMF (0.44 mL) at room temperature was added (R)-23-(4-(4-(6-amino-5-(1-(2,6-dichloro-3-fluorophenyl)ethoxy)pyridin-3-yl)-1H-pyrazol-1-yl)piperidin-1-yl)-3,6,9,12,15,18,21-heptaoxatricosanoic acid (35 mg, 0.04 mmol). The reaction mixture was stirred at room temperature. After 2 h, LCMS showed complete conversion. The reaction mixture was loaded directly onto a 30 g gold C18 column to purify by reverse phase column chromatography (5% to 100% MeCN in water, 0.1% formic acid). The fractions were combined and concentrated, followed by a catch and release purification on an SPE cartridge (tosic acid) to afford 25 mg of the desired product in 66% purity. It was then purified by preparative HPLC to afford 16.7 mg of an off-white solid as the desired product in 31.2% yield.

LC/MS Method 3: retention time: 2.456 min, 98.7% purity at 215 nm, [M+2H]/2⁺=614.8 615.7.

¹H NMR (400 MHz, DMSO-d₆) δ ppm 8.98 (s, 1H), 8.56-8.62 (m, 1H), 7.92-7.97 (m, 1H), 7.74-7.77 (m, 1H), 7.52-7.59 (m, 2H), 7.36-7.48 (m, 6H), 6.87-6.91 (m, 1H), 6.05-6.11 (m, 1H), 5.60-5.69 (m, 2H), 5.13-5.18 (m, 1H), 4.54-4.59 (m, 1H), 4.32-4.48 (m, 3H), 4.20-4.29 (m, 1H), 4.07-4.17 (m, 1H), 3.94-4.00 (m, 2H), 3.46-3.69 (m, 30H), 2.85-3.19 (m, 2H), ), 2.44 (s, 3H), 1.86-2.12 (m, 6H), 1.80 (d, J=6.6 Hz, 3H), 0.94 (s, 9H). Two aliphatic protons missing. Impurities on the ¹H NMR spectrum between 1.09 and 1.23 ppm.

### Example S8. Preparation of N-(2-(4-(4-(6-amino-5-((R)-1-(2,6-dichloro-3-fluorophenyl)ethoxy)pyridin-3-yl)-1H-pyrazol-1-yl)piperidin-1-yl)ethyl)-2-((2-(2,6-dioxopiperidin-3-yl)-1,3-dioxoisoindolin-4-yl)oxy)acetamide (Compound 8)

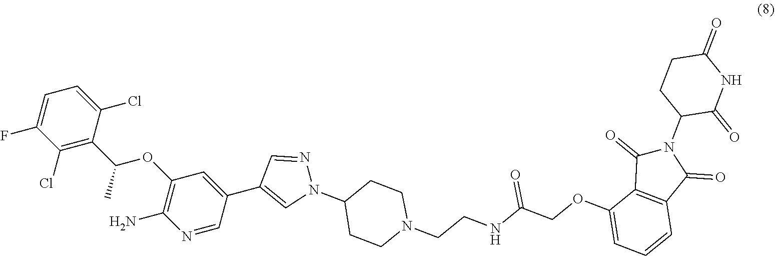 [SMILES: C[C@@H](Oc1cc(-c2cnn(C3CCN(CCNC(=O)COc4cccc5c4C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-4-yl)oxy)-N-(2-hydroxyethyl)acetamide (20 mg, 0.05 mmol) in DMA (0.85 mL) at room temperature was added Dess-Martin periodinane (38 mg, 0.091 mmol) in one portion. The resulting solution was stirred at room temperature. The solution was stirred at room temperature overnight. LCMS showed complete oxidation. (R)-3-(1-(2,6-dichloro-3-fluorophenyl)ethoxy)-5-(1-(piperidin-4-yl)-1H-pyrazol-4-yl)pyridin-2-amine (24 mg, 0.05 mmol) was added, followed by sodium triacetoxyborohydride (17 mg, 0.08 mmol). The resulting mixture was stirred at room temperature. After 2 h, the mixture showed full conversion to the desired product. The reaction mixture was purified using reverse phase column chromatography (5% to 100% MeCN in water, 0.1% formic acid). The fractions were combined and concentrated to afford 14 mg of the desired product as a yellow solid.

LC/MS Method 6: retention time: 3.183 min, 91.7% purity at 215 nm, [M+2H]/2⁺=404.1.

¹H NMR (400 MHz, DMSO-d₆) δ ppm 11.10-11.15 (m, 1H), 8.13 (s, 1H), 7.95 (br s, 1H), 7.79-7.88 (m, 1H), 7.74-7.79 (m, 1H), 7.55-7.61 (m, 2H), 7.51 (d, J=7.3 Hz, 1H), 7.42-7.48 (m, 2H), 6.91 (s, 1H), 6.04-6.14 (m, 1H), 5.65-5.79 (m, 2H), 5.07-5.16 (m, 1H), 4.85 (br s, 2H), 3.46-3.58 (m, 2H), 2.81-2.95 (m, 2H), 2.09-2.29 (m, 4H), 1.97-2.08 (m, 2H), 1.80 (d, J=6.8 Hz, 3H). Seven aliphatic protons missing on the ¹H NMR.

### Example S9. Preparation of N-(2-(2-(4-(4-(6-amino-5-((R)-1-(2,6-dichloro-3-fluorophenyl)ethoxy)pyridin-3-yl)-1H-pyrazol-1-yl)piperidin-1-yl)ethoxy)ethyl)-2-((2-(2,6-dioxopiperidin-3-yl)-1,3-dioxoisoindolin-4-yl)oxy)acetamide (Compound 9)

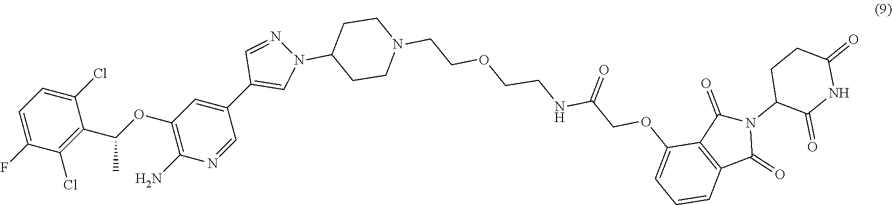 [SMILES: C[C@@H](Oc1cc(-c2cnn(C3CCN(CCOCCNC(=O)COc4cccc5c4C(=O)N(C4CCC(=O)NC4=O)C5=O)CC3)c2)cnc1N)c1c(Cl)ccc(F)c1Cl]

To a solution of (R)-3-(1-(2,6-dichloro-3-fluorophenyl)ethoxy)-5-(1-(piperidin-4-yl)-1H-pyrazol-4-yl)pyridin-2-amine (22 mg, 0.05 mmol) in DMSO (0.34 mL) at room temperature was added 2-((2-(2,6-dioxopiperidin-3-yl)-1,3-dioxoisoindolin-4-yl)oxy)-N-(2-(2-hydroxyethoxy)ethyl)acetamide (20 mg, 0.05 mmol). The resulting solution was stirred at room temperature overnight. LCMS showed 50-55% conversion. Then, the solution of aldehyde was added dropwise over 3 min to a solution of IBX (17 mg, 0.06 mmol) and sodium triacetoxyborohydride (13 mg, 0.06 mmol) in DCE (0.4 mL). The resulting mixture was stirred at room temperature. After 15 min, LCMS showed full conversion of aldehyde to the desired product. The reaction mixture was purified by reverse phase column chromatography (5% to 100% MeCN in water, 0.1% formic acid). The fractions were combined and concentrated under reduced pressure to afford 13.09 mg of the desired product in 29% yield as a light yellow solid.

LCMS Method 6: retention time: 3.175 min, 89.1% purity at 215 nm, [M+H]⁺=851.2, 853.2.

¹H NMR (300 MHz, DMSO-d₆) δ ppm 11.07 (s, 1H), 7.91 (br t, J=5.6 Hz, 1H), 7.86 (s, 1H), 7.71-7.78 (m, 1H), 7.68 (d, J=1.5 Hz, 1H), 7.51 (dd, J=8.8, 5.0 Hz, 1H), 7.44 (s, 1H), 7.41 (d, J=3.8 Hz, 1H), 7.32-7.39 (m, 2H), 6.82 (d, J=1.5 Hz, 1H), 6.01 (q, J=6.9 Hz, 1H), 5.58 (s, 2H), 5.05 (dd, J=12.8, 5.4 Hz, 1H), 4.73 (s, 2H), 3.92-4.06 (m, 1H), 3.35-3.53 (m, 6H), 2.75-2.94 (m, 3H), 2.48-2.58 (m, 2H), 1.78-2.11 (m, 7H), 1.71-1.77 (m, 3H). Two aliphatic protons missing in ¹H NMR. Contains traces of formic acid (1.6% w/w) by ¹H NMR.

### Example S10. Preparation of N-(2-(2-(2-(4-(4-(6-amino-5-((R)-1-(2,6-dichloro-3-fluorophenyl)ethoxy)pyridin-3-yl)-1H-pyrazol-1-yl)piperidin-1-yl)ethoxy)ethoxy)ethyl)-2-((2-(2,6-dioxopiperidin-3-yl)-1,3-dioxoisoindolin-4-yl)oxy)acetamide (Compound 10)

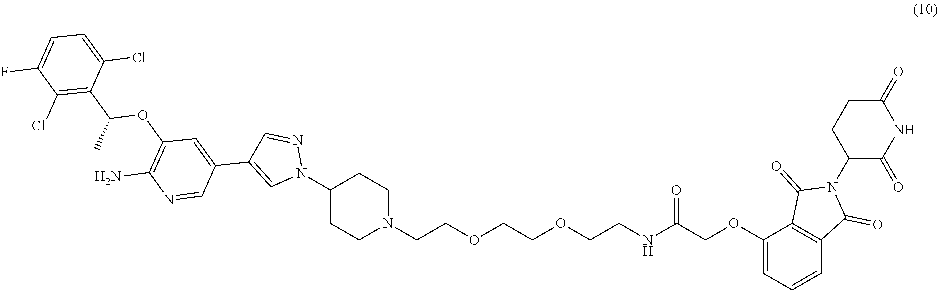 [SMILES: C[C@@H](Oc1cc(-c2cnn(C3CCN(CCOCCOCCNC(=O)COc4cccc5c4C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-4-yl)oxy)-N-(2-(2-(2-hydroxyethoxy)ethoxy)ethyl)acetamide (20 mg, 0.04 mmol) in DMSO (0.40 mL) at room temperature was added IBX (15.7 mg, 0.06 mmol) in one portion and the solution was stirred at room temperature overnight. LCMS showed 50-55% conversion. The solution of aldehyde was added dropwise over 3 min to a solution (R)-3-(1-(2,6-dichloro-3-fluorophenyl)ethoxy)-5-(1-(piperidin-4-yl)-1H-pyrazol-4-yl)pyridin-2-amine (20 mg, 0.0400 mmol) and sodium triacetoxyborohydride (11.9 mg, 0.0600 mmol) in DCE (0.44 mL) The resulting mixture was stirred at room temperature. After 15 min, LCMS showed full conversion of aldehyde to the desired product. The reaction mixture was purified by reverse phase column chromatography (5% to 100% MeCN in water, 0.1% formic acid). The fractions were combined and concentrated to afford 7.97 mg of the desired product in 20% yield as a light yellow solid.

¹H NMR (400 MHz, DMSO-d₆) δ ppm 11.13 (s, 1H), 7.99 (t, J=5.6 Hz, 1H), 7.88-7.95 (m, 1H), 7.80 (dd, J=8.6, 7.3 Hz, 1H), 7.74 (s, 1H), 7.57 (dd, J=8.9, 5.0 Hz, 1H), 7.36-7.53 (m, 4H), 6.89 (d, J=1.5 Hz, 1H), 6.03-6.12 (m, 1H), 5.63 (s, 2H), 5.05-5.17 (m, 1H), 4.79 (s, 2H), 4.00-4.12 (m, 1H), 3.34-3.60 (m, 12H), 2.83-3.01 (m, 3H), 2.54-2.64 (m, 2H), 1.86-2.15 (m, 7H), 1.80 (d, J=6.6 Hz, 3H).

LC/MS Method 6: retention time: 3.201 min, 97.8% purity at 215 nm, [M+H]⁺=895.2, 897.2.

### Example S11. Preparation of N-(2-(2-(2-(2-(4-(4-(6-amino-5-((R)-1-(2,6-dichloro-3-fluorophenyl)ethoxy)pyridin-3-yl)-1H-pyrazol-1-yl)piperidin-1-yl)ethoxy)ethoxy)ethoxy)ethyl)-2-((2-(2,6-dioxopiperidin-3-yl)-1,3-dioxoisoindolin-4-yl)oxy)acetamide (Compound 11)

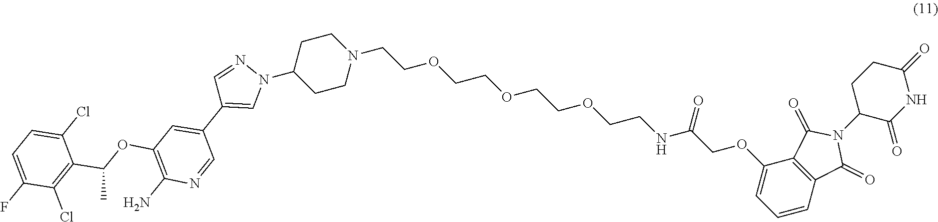 [SMILES: C[C@@H](Oc1cc(-c2cnn(C3CCN(CCOCCOCCOCCNC(=O)COc4cccc5c4C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-4-yl)oxy)-N-(2-(2-(2-(2-hydroxyethoxy)ethoxy)ethoxy)ethyl)acetamide (20 mg, 0.04 mmol) in DMSO (0.37 mL) at room temperature was added IBX (14.4 mg, 0.05 mmol)) in one portion. The resulting solution was stirred at room temperature overnight. The solution of aldehyde was added dropwise over 3 min to a solution of (R)-3-(1-(2,6-dichloro-3-fluorophenyl)ethoxy)-5-(1-(piperidin-4-yl)-1H-pyrazol-4-yl)pyridin-2-amine (18.4 mg, 0.04 mmol) and sodium triacetoxyborohydride (10.9 mg, 0.05 mmol) in DCE (0.40 mL). The resulting mixture was stirred at room temperature. After 15 min, LCMS showed full conversion of aldehyde to the desired product. The reaction mixture was purified by reverse phase column chromatography (5% to 100% MeCN in water, 0.1% formic acid). The fractions were combined and concentrated to afford 9.13 mg of the desired product in 23% yield as a light yellow solid.

¹H NMR (300 MHz, DMSO-d₆) δ ppm 11.13 (s, 1H), 8.00 (br t, J=5.7 Hz, 1H), 7.94 (s, 1H), 7.77-7.85 (m, 1H), 7.74 (s, 1H), 7.57 (dd, J=9.1, 5.0 Hz, 1H), 7.46-7.53 (m, 2H), 7.41 (t, J=8.5 Hz, 2H), 6.88 (d, J=1.5 Hz, 1H), 6.08 (q, J=6.7 Hz, 1H), 5.64 (s, 2H), 5.11 (dd, J=12.8, 5.4 Hz, 1H), 4.78 (s, 2H), 4.00-4.13 (m, 1H), 3.42-3.57 (m, 16H), 2.82-3.00 (m, 3H), 2.55-2.64 (m, 2H), 1.84-2.19 (m, 7H), 1.80 (d, J=6.5 Hz, 3H).

LC/MS Method 6: retention time: 3.211 min, 92.6% purity at 215 nm, [M+H]⁺=939.1 941.0.

### Example S12. Preparation of N-(14-(4-(4-(6-amino-5-((R)-1-(2,6-dichloro-3-fluorophenyl)ethoxy)pyridin-3-yl)-1H-pyrazol-1-yl)piperidin-1-yl)-3,6,9,12-tetraoxatetradecyl)-2-((2-(2,6-dioxopiperidin-3-yl)-1,3-dioxoisoindolin-4-yl)oxy)acetamide (Compound 12)

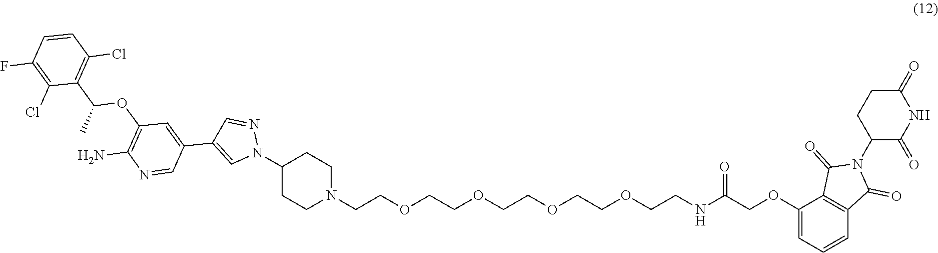 [SMILES: C[C@@H](Oc1cc(-c2cnn(C3CCN(CCOCCOCCOCCOCCNC(=O)COc4cccc5c4C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-4-yl)oxy)-N-(14-hydroxy-3,6,9,12-tetraoxatetradecyl)acetamide (20 mg, 0.036 mmol) in DMSO (340 μL) at room temperature was added IBX (13.2 mg, 0.047 mmol) in one portion. The resulting solution was stirred at room temperature overnight. LCMS analysis showed 50-55% conversion to the desired aldehyde. The crude solution of aldehyde was added dropwise over 3 min to a solution of (R)-3-(1-(2,6-dichloro-3-fluorophenyl)ethoxy)-5-(1-(piperidin-4-yl)-1H-pyrazol-4-yl)pyridin-2-amine (16.9 mg, 0.036 mmol) and sodium triacetoxyborohydride (10 mg, 0.047 mmol) in DCE (400 μL). The resulting mixture was stirred at room temperature for 15 min. LCMS analysis showed full conversion of the aldehyde to the desired product. The reaction mixture was purified by reverse phase column chromatography (5% to 100% MeCN in water, 0.1% formic acid). The resulting material was further purified by preparative LCMS, affording the product, N-(14-(4-(4-(6-amino-5-((R)-1-(2,6-dichloro-3-fluorophenyl)ethoxy)pyridin-3-yl)-1H-pyrazol-1-yl)piperidin-1-yl)-3,6,9,12-tetraoxatetradecyl)-2-((2-(2,6-dioxopiperidin-3-yl)-1,3-dioxoisoindolin-4-yl)oxy)acetamide (9 mg, 0.0087 mmol, 24% yield), as a light-yellow solid after lyophilization.

¹H NMR (400 MHz, DMSO-d₆) δ ppm 11.12 (s, 1H), 7.99 (t, J=4.9 Hz, 1H), 7.94 (s, 1H), 7.78-7.84 (m, 1H), 7.74 (d, J=1.7 Hz, 1H), 7.54-7.60 (m, 1H), 7.37-7.52 (m, 4H), 6.89 (d, J=1.7 Hz, 1H), 6.08 (q, J=6.9 Hz, 1H), 5.63 (s, 2H), 5.11 (dd, J=13.0, 5.4 Hz, 1H), 4.78 (s, 2H), 4.00-4.14 (m, 1H), 3.43-3.57 (m, 20H), 2.83-3.01 (m, 3H), 2.54-2.68 (m, 2H), 1.84-2.19 (m, 7H), 1.80 (d, J=6.6 Hz, 3H).

LC/MS Method 6: MS (ESI) [M+2H]/2⁺ 493.1, rt: 3.214 min.

### Example S13. Preparation of N-(2-(4-(4-(6-amino-5-((R)-1-(2,6-dichloro-3-fluorophenyl)ethoxy)pyridin-3-yl)-1H-pyrazol-1-yl)piperidin-1-yl)ethyl)-2-((2-(2,6-dioxopiperidin-3-yl)-1,3-dioxoisoindolin-5-yl)oxy)acetamide (Compound 13)

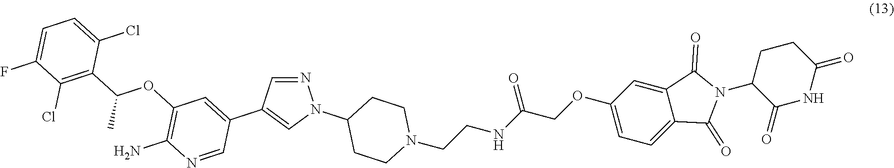 [SMILES: C[C@@H](Oc1cc(-c2cnn(C3CCN(CCNC(=O)COc4ccc5c(c4)C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-5-yl)oxy)-N-(2-hydroxyethyl)acetamide (20 mg, 0.05 mmol) in DMA (0.85 mL) at room temperature was added Dess-Martin periodinane (38.4 mg, 0.091 mmol) in one portion. The resulting solution was stirred at room temperature overnight. LCMS showed that the oxidation was mostly over. (R)-3-(1-(2,6-dichloro-3-fluorophenyl)ethoxy)-5-(1-(piperidin-4-yl)-1H-pyrazol-4-yl)pyridin-2-amine (24 mg, 0.05 mmol) was added, followed by sodium triacetoxyborohydride (16.9 mg, 0.08 mmol). The resulting mixture was stirred at room temperature for 1 h. The reaction mixture was purified by reverse phase column chromatography (5% to 100% MeCN in water, 0.1% formic acid). The fractions were combined and concentrated to give 7.04 mg of the desired product in 16% yield as a off-white solid.

LC/MS Method 6: retention time: 3.176 min, 98.0% purity at 215 nm, [M+H]⁺=807.1, [M+2H]/2⁺=404.8.

¹H NMR (400 MHz, DMSO-d₆) δ ppm 11.11 (s, 1H), 7.94 (s, 1H), 7.88 (d, J=8.3 Hz, 1H), 7.75 (s, 1H), 7.52-7.61 (m, 2H), 7.38-7.48 (m, 4H), 6.90 (d, J=1.5 Hz, 1H), 6.08 (d, J=6.8 Hz, 1H), 5.65 (br. s, 2H), 5.08-5.16 (m, 1H), 4.75 (s, 2H), 2.92 (s, 4H), 2.04 (s, 6H), 1.80 (d, J=6.6 Hz, 3H). Seven aliphatic protons missing in the ¹H NMR spectrum. Contains traces of formic acid (2.2% w/w) by ¹H NMR.

### Example S14. Preparation of N-(2-(2-(4-(4-(6-amino-5-((R)-1-(2,6-dichloro-3-fluorophenyl)ethoxy)pyridin-3-yl)-1H-pyrazol-1-yl)piperidin-1-yl)ethoxy)ethyl)-2-((2-(2,6-dioxopiperidin-3-yl)-1,3-dioxoisoindolin-5-yl)oxy)acetamide (Compound 14)

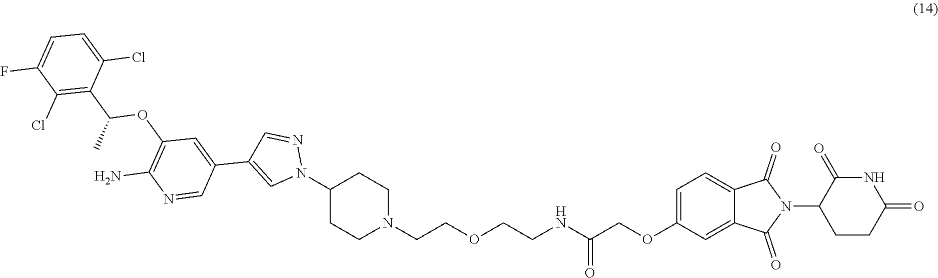 [SMILES: C[C@@H](Oc1cc(-c2cnn(C3CCN(CCOCCNC(=O)COc4ccc5c(c4)C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-5-yl)oxy)-N-(2-(2-hydroxyethoxy)ethyl)acetamide (20 mg, 0.050 mmol) in DMSO (0.45 mL) at room temperature was added IBX (17.3 mg, 0.060 mmol) in one portion and the solution was stirred at room temperature overnight. The solution of aldehyde was added dropwise over 3 min to a solution of (R)-3-(1-(2,6-dichloro-3-fluorophenyl)ethoxy)-5-(1-(piperidin-4-yl)-1H-pyrazol-4-yl)pyridin-2-amine (22.1 mg, 0.050 mmol) and sodium triacetoxyborohydride (13.1 mg, 0.060 mmol) in DCE (0.48 mL). The resulting mixture was stirred at room temperature. After 15 min, full conversion of aldehyde to the desired product was observed. The reaction mixture was purified by reverse phase column chromatography (5% to 100% MeCN in water, 0.1% formic acid). The fractions were combined and concentrated to give 9.09 mg of the desired product in 22% yield as a light yellow solid.

LC/MS Method 6: retention time: 3.158 min, 97.6% purity at 215 nm, [M+H]⁺=851.2, 853.1.

¹H NMR (400 MHz, DMSO-d₆) δ ppm 11.11 (s, 1H), 8.24 (br t, J=5.4 Hz, 1H), 7.92-7.96 (m, 1H), 7.86 (d, J=8.3 Hz, 1H), 7.74 (s, 1H), 7.57 (dd, J=9.0, 4.9 Hz, 1H), 7.51 (s, 1H), 7.34-7.48 (m, 3H), 6.89 (d, J=1.7 Hz, 1H), 6.08 (d, J=6.8 Hz, 1H), 5.64 (s, 2H), 5.05-5.17 (m, 1H), 4.70-4.79 (m, 2H), 3.98-4.12 (m, 1H), 3.36-3.57 (m, 8H), 2.84-2.99 (m, 3H), 2.54-2.63 (m, 2H), 1.86-2.17 (m, 7H), 1.80 (d, J=6.6 Hz, 3H).

### Example S15. Preparation of N-(2-(2-(2-(4-(4-(6-amino-5-((R)-1-(2,6-dichloro-3-fluorophenyl)ethoxy)pyridin-3-yl)-1H-pyrazol-1-yl)piperidin-1-yl)ethoxy)ethoxy)ethyl)-2-((2-(2,6-dioxopiperidin-3-yl)-1,3-dioxoisoindolin-5-yl)oxy)acetamide (Compound 15)

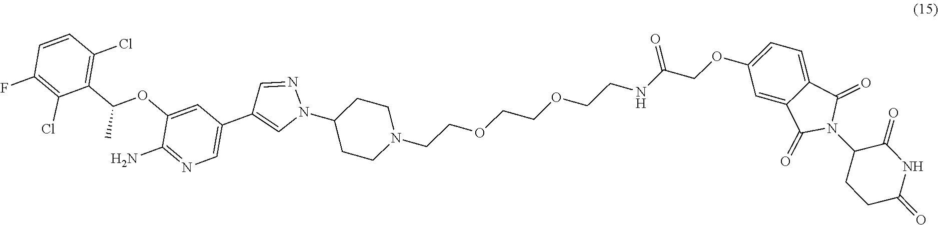 [SMILES: C[C@@H](Oc1cc(-c2cnn(C3CCN(CCOCCOCCNC(=O)COc4ccc5c(c4)C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-5-yl)oxy)-N-(2-(2-(2-hydroxyethoxy)ethoxy)ethyl)acetamide (20 mg, 0.043 mmol) in DMSO (255 μL) at room temperature was added IBX (15.7 mg, 0.056 mmol) in one portion and the solution was stirred at room temperature overnight. LCMS analysis revealed 50% conversion. The crude solution of aldehyde was added dropwise over 3 min to a solution of (R)-3-(1-(2,6-dichloro-3-fluorophenyl)ethoxy)-5-(1-(piperidin-4-yl)-1H-pyrazol-4-yl)pyridin-2-amine (20 mg, 0.045 mmol) and sodium triacetoxyborohydride (11.9 mg, 0.056 mmol) in DCE (340 L). The resulting mixture was stirred at room temperature for 15 min. The reaction mixture was purified by reverse phase column chromatography (5% to 100% MeCN in water, 0.1% formic acid). The resulting material was further purified by preparative LCMS, affording the product, N-(2-(2-(2-(4-(4-(6-amino-5-((R)-1-(2,6-dichloro-3-fluorophenyl)ethoxy)pyridin-3-yl)-1H-pyrazol-1-yl)piperidin-1-yl)ethoxy)ethoxy)ethyl)-2-((2-(2,6-dioxopiperidin-3-yl)-1,3-dioxoisoindolin-5-yl)oxy)acetamide (11.1 mg, 0.012 mmol, 28% yield), as a white solid after lyophilization.

¹H NMR (400 MHz, DMSO-d₆) δ ppm 11.08-11.19 (m, 1H), 8.23-8.28 (m, 1H), 7.94 (s, 1H), 7.86 (d, J=8.3 Hz, 1H), 7.75 (d, J=1.5 Hz, 1H), 7.57 (dd, J=8.9, 5.0 Hz, 1H), 7.52 (s, 1H), 7.42-7.47 (m, 2H), 7.38 (dd, J=8.3, 2.2 Hz, 1H), 6.89 (d, J=1.5 Hz, 1H), 6.08 (q=J=6.4 Hz, 1H), 5.6 (s, 2H), 5.10-5.15 (m, 1H), 4.74 (s, 2H), 4.03-4.11 (m, 1H), 3.51 (s, 10H), 3.29-3.33 (m, 2H), 2.83-2.96 (m, 3H), 2.54-2.64 (m, 2H), 2.12 (d, J=2.4 Hz, 3H) 1.94 (s, 4H), 1.80 (d, J=6.6 Hz, 3H).

LC/MS Method 3: MS (ESI) [M+H]⁺ 897.3, rt: 2.154 min.

### Example S16. Preparation of N-(2-(2-(2-(2-(4-(4-(6-amino-5-((R)-1-(2,6-dichloro-3-fluorophenyl)ethoxy)pyridin-3-yl)-1H-pyrazol-1-yl)piperidin-1-yl)ethoxy)ethoxy)ethoxy)ethyl)-2-((2-(2,6-dioxopiperidin-3-yl)-1,3-dioxoisoindolin-5-yl)oxy)acetamide (Compound 16)

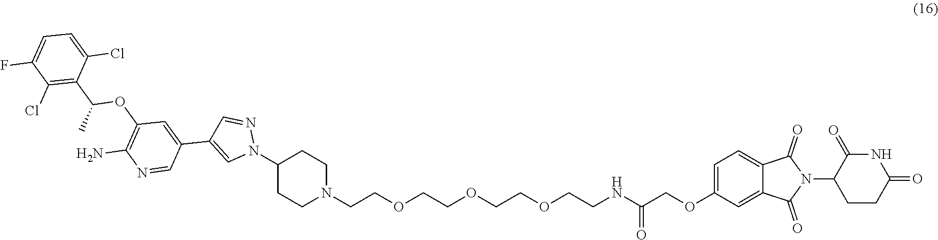 [SMILES: C[C@@H](Oc1cc(-c2cnn(C3CCN(CCOCCOCCOCCNC(=O)COc4ccc5c(c4)C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-5-yl)oxy)-N-(2-(2-(2-(2-hydroxyethoxy)ethoxy)ethoxy)ethyl)acetamide (20 mg, 0.040 mmol) in DMSO (0.30 mL) at room temperature was added IBX (14.4 mg, 0.050 mmol) in one portion and the solution was stirred at room temperature overnight. The solution of aldehyde was added dropwise over 3 min to a solution of (R)-3-(1-(2,6-dichloro-3-fluorophenyl)ethoxy)-5-(1-(piperidin-4-yl)-1H-pyrazol-4-yl)pyridin-2-amine (18.4 mg, 0.040 mmol) and sodium triacetoxyborohydride (10.9 mg, 0.050 mmol) in DCE (0.40 mL). The resulting mixture was stirred at room temperature. After 15 min, LCMS showed full conversion of aldehyde to the desired product. The reaction mixture was purified by reverse column chromatography (5% to 100% MeCN in water, 0.1% formic acid). The fractions were combined and concentrated to afford 6.1 mg of the desired product in 15% yield as a light yellow solid.

¹H NMR (300 MHz, DMSO-d₆) δ ppm 11.12 (s, 1H), 8.20-8.30 (m, 1H), 7.93-7.97 (m, 1H), 7.86 (d, J=8.5 Hz, 1H), 7.74 (d, J=1.5 Hz, 1H), 7.36-7.63 (m, 5H), 6.89 (d, J=1.5 Hz, 1H), 6.01-6.14 (m, 1H), 5.64 (s, 2H), 5.12 (dd, J=12.8, 5.4 Hz, 1H), 4.67-4.80 (m, 2H), 3.98-4.16 (m, 1H), 3.42-3.64 (m, 16H), 2.84-3.03 (m, 3H), 2.58-2.76 (m, 2H), 1.86-2.20 (m, 7H), 1.80 (d, J=6.8 Hz, 3H).

LC/MS Method 6: retention time: 3.190 min, 91.9% purity at 215 nm, [M+H]⁺=939.1, 941.0.

### Example 17. Preparation of N-(14-(4-(4-(6-amino-5-((R)-1-(2,6-dichloro-3-fluorophenyl)ethoxy)pyridin-3-yl)-1H-pyrazol-1-yl)piperidin-1-yl)-3,6,9,12-tetraoxatetradecyl)-2-((2-(2,6-dioxopiperidin-3-yl)-1,3-dioxoisoindolin-5-yl)oxy)acetamide (Compound 17)

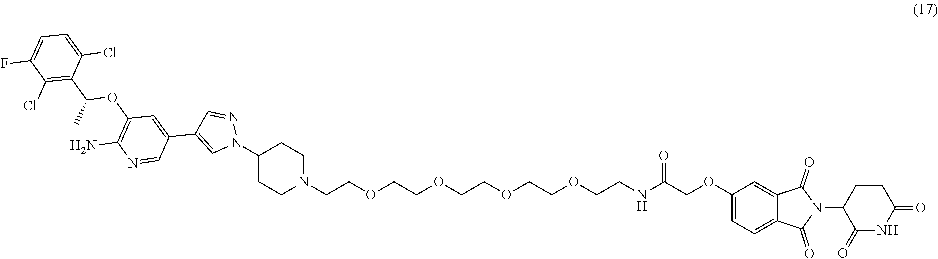 [SMILES: C[C@@H](Oc1cc(-c2cnn(C3CCN(CCOCCOCCOCCOCCNC(=O)COc4ccc5c(c4)C(=O)N(C4CCC(=O)NC4=O)C5=O)CC3)c2)cnc1N)c1c(Cl)ccc(F)c1Cl]

To a solution of 2-((2-(2,6-dioxopiperidin-3-yl)-1,3-dioxoisoindolin-5-yl)oxy)-N-(14-hydroxy-3,6,9,12-tetraoxatetradecyl)acetamide (20 mg, 0.040 mmol) in DMSO (0.30 L) at room temperature was added IBX (13.2 mg, 0.050 mmol) in one portion and the solution was stirred at room temperature overnight. The solution of aldehyde was added dropwise over 3 min to a solution of (R)-3-(1-(2,6-dichloro-3-fluorophenyl)ethoxy)-5-(1-(piperidin-4-yl)-1H-pyrazol-4-yl)pyridin-2-amine (16.9 mg, 0.040 mmol) and sodium triacetoxyborohydride (9.99 mg, 0.050 mmol) in DCE (0.40 mL). The resulting mixture was stirred at room temperature. After 15 min, LCMS showed full conversion of aldehyde to the desired product. The reaction mixture was purified by reverse column chromatography (5% to 100% MeCN in water, 0.1% formic acid). The fractions were combined and concentrated to afford 5.48 mg of the desired product in 14.0% yield as a light yellow solid.

¹H NMR (300 MHz, DMSO-d₆) δ ppm 11.12 (s, 1H), 8.20-8.34 (m, 1H), 7.92-7.98 (m, 1H), 7.86 (d, J=8.2 Hz, 1H), 7.74 (d, J=1.5 Hz, 1H), 7.34-7.61 (m, 5H), 6.89 (d, J=1.5 Hz, 1H), 6.08 (d, J=7.0 Hz, 1H), 5.64 (s, 2H), 5.03-5.25 (m, 1H), 4.63-4.84 (m, 2H), 3.97-4.17 (m, 1H), 3.39-3.68 (m, 20H), 2.79-3.00 (m, 3H), 2.58-2.75 (m, 2H), 1.85-2.20 (m, 7H), 1.80 (d, J=6.8 Hz, 3H).

LC/MS Method 6: retention time: 3.194 min, 93.6% purity at 215 nm, [M+2H]²⁺=92.9.

### Example S18. Preparation of 5-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)-2-(2,6-dioxopiperidin-3-yl)isoindoline-1,3-dione (Compound 18)

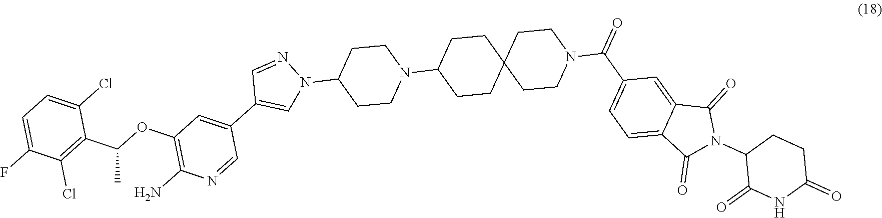 [SMILES: C[C@@H](Oc1cc(-c2cnn(C3CCN(C4CCC5(CC4)CCN(C(=O)c4ccc6c(c4)C(=O)N(C4CCC(=O)NC4=O)C6=O)CC5)CC3)c2)cnc1N)c1c(Cl)ccc(F)c1Cl]

To a solution of (R)-5-(1-(1-(3-azaspiro[5.5]undecan-9-yl)piperidin-4-yl)-1H-pyrazol-4-yl)-3-(1-(2,6-dichloro-3-fluorophenyl)ethoxy)pyridin-2-amine (bis-hydrochloride) (50 mg, 0.07 mmol) in DMF (750 μL) was added 2-(2,6-dioxo-3-piperidyl)-1,3-dioxo-isoindoline-5-carboxylic acid (22 mg, 0.07 mmol), DIPEA (60 μL, 0.37 mmol), and HATU (42 mg, 0.11 mmol). The reaction was stirred under N₂ for 1 h and then purified by reverse phase column chromatography (5 to 100% MeCN in water, 0.1% formic acid) to afford mixed fractions, which were recombined and re-purified by reverse phase column chromatography (5 to 100% MeCN in water) to afford the product, 5-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)-2-(2,6-dioxopiperidin-3-yl)isoindoline-1,3-dione (6.3 mg, 9% yield), as a white solid after lyophilization.

¹H NMR (400 MHz, DMSO-d₆) δ ppm 11.15 (s, 1H), 7.97 (br dd, J=15.7, 7.1 Hz, 2H), 7.80-7.91 (m, 2H), 7.74 (br s, 1H), 7.49-7.61 (m, 2H), 7.44 (br t, J=8.9 Hz, 1H), 6.89 (br, s, 1H), 6.08 (br d, J=6.1 Hz, 1H), 5.63 (br s, 2H), 5.12-5.23 (m, 1H), 3.98-4.17 (m, 1H), 3.57-3.70 (m, 2H), 3.24 (br d, J=2.4 Hz, 2H), 2.83-3.05 (m, 3H), 2.55-2.70 (m, 2H), 2.26-2.42 (m, 3H), 1.95-2.12 (m, 3H), 1.84-1.93 (m, 2H), 1.76-1.83 (m, 4H), 1.53-1.67 (m, 3H), 1.31-1.49 (m, 4H), 1.19-1.31 (m, 2H), 1.04-1.19 (m, 2H). LC/MS Method 3: MS (ESI) [M+2H]/2⁺ 444.0, rt: 2.124 min.

### Example S19. Preparation of 3-(6-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)-1-oxoisoindolin-2-yl)piperidine-2,6-dione (Compound 19)

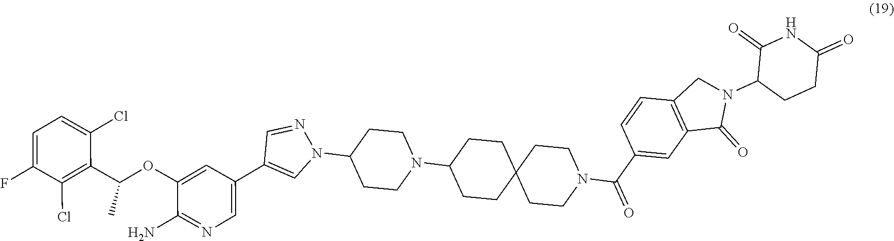 [SMILES: C[C@@H](Oc1cc(-c2cnn(C3CCN(C4CCC5(CC4)CCN(C(=O)c4ccc6c(c4)C(=O)N(C4CCC(=O)NC4=O)C6)CC5)CC3)c2)cnc1N)c1c(Cl)ccc(F)c1Cl]

HATU (11.2 mg, 0.03 mmol) and 2-(2,6-dioxo-3-piperidyl)-3-oxo-isoindoline-5-carboxylic acid (8.5 mg, 0.03 mmol) were added to a 1 mL vial equipped with a stir bar. DMF (350 μL) was then added, followed by DIPEA (11 μL, 0.11 mmol). The resulting reaction mixture was allowed to stir for 5 min, prior to the addition of (R)-5-(1-(1-(3-azaspiro[5.5]undecan-9-yl)piperidin-4-yl)-1H-pyrazol-4-yl)-3-(1-(2,6-dichloro-3-fluorophenyl)ethoxy)pyridin-2-amine (20 mg, 0.03 mmol). The vial was then sealed with a cap and allowed to stir overnight at room temperature. LCMS analysis revealed full conversion of the starting material to the desired product. The reaction mixture was then purified by mass-directed prep HPLC with formic acid modifier, affording the formic acid salt of the product, 3-(6-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)-1-oxoisoindolin-2-yl)piperidine-2,6-dione (10.6 mg, 0.12 mmol, 43% yield), as an off-white solid after lyophilization.

LC/MS Method 2: MS (ESI) [M+2H]/2⁺ 436.6, rt: 1.00 min.

### Example S20. Preparation of 3-(5-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)-1-oxoisoindolin-2-yl)piperidine-2,6-dione (Compound 20)

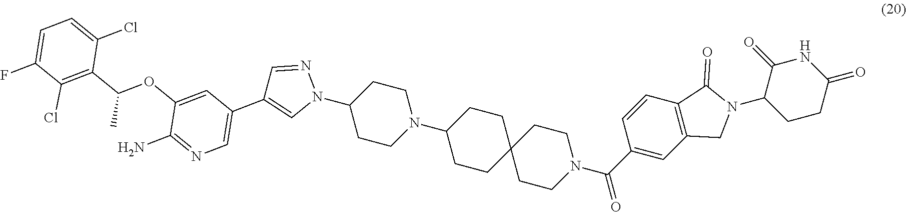 [SMILES: C[C@@H](Oc1cc(-c2cnn(C3CCN(C4CCC5(CC4)CCN(C(=O)c4ccc6c(c4)CN(C4CCC(=O)NC4=O)C6=O)CC5)CC3)c2)cnc1N)c1c(Cl)ccc(F)c1Cl]

HATU (11.2 mg, 0.03 mmol) and 2-(2,6-dioxo-3-piperidyl)-1-oxo-isoindoline-5-carboxylic acid (8.5 mg, 0.03 mmol) were added to a 1 mL vial equipped with a stir bar. DMF (350 μL) was then added, followed by DIPEA (11 μL, 0.11 mmol). The resulting reaction mixture was allowed to stir for 5 min, prior to the addition of (R)-5-(1-(1-(3-azaspiro[5.5]undecan-9-yl)piperidin-4-yl)-1H-pyrazol-4-yl)-3-(1-(2,6-dichloro-3-fluorophenyl)ethoxy)pyridin-2-amine (20 mg, 0.03 mmol). The vial was then sealed with a cap and allowed to stir overnight at room temperature. LCMS analysis revealed full conversion of the starting material to the desired product. The reaction mixture was then purified by mass-directed prep HPLC with formic acid modifier, affording the formic acid salt of the product, 3-(5-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)-1-oxoisoindolin-2-yl)piperidine-2,6-dione (13 mg, 0.14 mmol, 53% yield), as an off-white solid after lyophilization.

¹H NMR (400 MHz, DMSO-d₆) δ ppm 11.00 (s, 1H), 7.95 (br s, 1H), 7.78 (d, J=7.8 Hz, 1H), 7.74 (s, 1H), 7.61 (s, 1H), 7.57 (dd, J=8.9, 5.0 Hz, 1H), 7.47-7.53 (m, 2H), 7.41-7.47 (m, 1H), 6.89 (s, 1H), 6.08 (q, J=6.4 Hz, 1H), 5.63 (s, 2H), 5.13 (dd, J=13.2, 5.1 Hz, 1H), 4.50 (d, J=18.3 Hz, 1H), 4.37 (d, J=18.1 Hz, 1H), 4.04 (br s, 1H), 3.61 (br s, 2H), 3.17-3.28 (m, 2H), 2.85-3.00 (m, 3H), 2.55-2.65 (m, 1H), 2.31 (br s, 4H), 1.92-2.06 (m, 3H), 1.82-1.92 (m, 2H), 1.80 (d, J=6.6 Hz, 4H), 1.71-1.77 (m, 1H), 1.50-1.64 (m, 3H), 1.29-1.48 (m, 4H), 1.20-1.28 (m, 1H), 1.03-1.19 (m, 2H). LC/MS Method 2: MS (ESI) [M+2H]/2⁺ 436.5, rt: 1.00 min.

### Example S21. Preparation of 3-(4-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)-1-oxoisoindolin-2-yl)piperidine-2,6-dione (Compound 21)

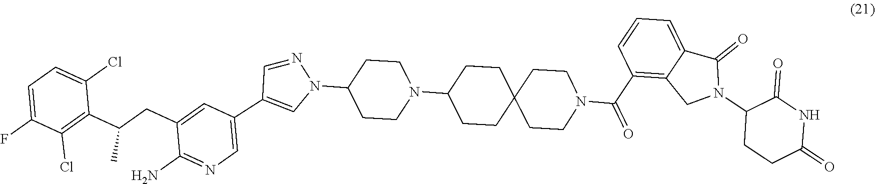 [SMILES: C[C@@H](Cc1cc(-c2cnn(C3CCN(C4CCC5(CC4)CCN(C(=O)c4cccc6c4CN(C4CCC(=O)NC4=O)C6=O)CC5)CC3)c2)cnc1N)c1c(Cl)ccc(F)c1Cl]

HATU (11.2 mg, 0.03 mmol) and 2-(2,6-dioxo-3-piperidyl)-1-oxo-isoindoline-4-carboxylic acid (8.5 mg, 0.03 mmol) were added to a 1 mL vial equipped with stir bar. DMF (350 μL) was then added followed by DIPEA (11 μL, 0.11 mmol). The resulting reaction mixture was allowed to stir for 5 min, prior to the addition of (R)-5-(1-(1-(3-azaspiro[5.5]undecan-9-yl)piperidin-4-yl)-1H-pyrazol-4-yl)-3-(1-(2,6-dichloro-3-fluorophenyl)ethoxy)pyridin-2-amine (20 mg, 0.03 mmol). The vial was then sealed with a cap and allowed to stir overnight at room temperature. LCMS analysis revealed full conversion of the starting material to the desired product. The reaction mixture was then purified by mass-directed prep HPLC with formic acid modifier, affording the formic acid salt of the product, 3-(4-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)-1-oxoisoindolin-2-yl)piperidine-2,6-dione (13 mg, 0.14 mmol, 52% yield), as an off-white solid after lyophilization.

LC/MS Method 2: MS (ESI) [M+2H]/2⁺ 436.6, rt: 1.01 min.

### Example S22. Preparation of 3-((3-(9-(4-(4-(6-Amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)phenyl)amino)piperidine-2,6-dione (Compound 22)

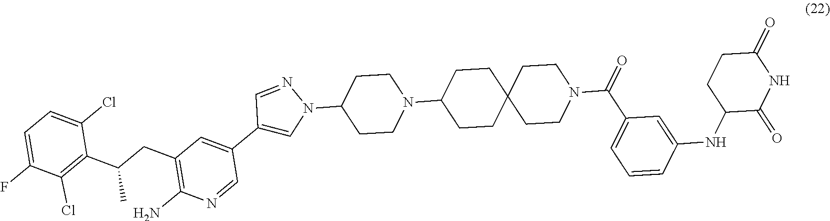 [SMILES: C[C@@H](Cc1cc(-c2cnn(C3CCN(C4CCC5(CC4)CCN(C(=O)c4cccc(NC6CCC(=O)NC6=O)c4)CC5)CC3)c2)cnc1N)c1c(Cl)ccc(F)c1Cl]

To a solution of 3-[(2,6-dioxo-3-piperidyl)amino]benzoic acid (502 mg, 2.0 mmol) in DMF (8 mL) at room temperature was added DIPEA (1.6 mL, 9.2 mmol) and 5-[1-[1-(3-azaspiro[5.5]undecan-9-yl)-4-piperidyl]pyrazol-4-yl]-3-[(1R)-1-(2,6-dichloro-3-fluoro-phenyl)ethoxy]pyridin-2-amine hydrochloride (1.17 g, 1.84 mmol). The resulting solution was stirred at room temperature for 10 minutes. Then, PyAOP (1.15 g, 2.21 mmol) was added in one portion. The reaction mixture was stirred at room temperature. After 3 h, LCMS showed full conversion. The mixture was left at room temperature overnight. The mixture was then purified by reverse phase column chromatography (5% MeCN to 100% MeCN in water with 0.1% HCOOH), affording 3-((3-(9-(4-(4-(6-amino-5-((R)-1-(2,6-dichloro-3-fluorophenyl)ethoxy)pyridin-3-yl)-1H-pyrazol-1-yl)piperidin-1-yl)-3-azaspiro[5.5]undecane-3-carbonyl)phenyl)amino)piperidine-2,6-dione as a formic acid salt (1:1.2 ratio, 370 mg, 0.44 mmol, 24% yield) as a light yellow solid.

LC/MS Method 3: MS (ESI) [M+H]⁺ 831.2, rt: 2.26 min.

### Biological Examples

### Example B1. IRAK3-ePL Overexpressing Degradation Assay

Stable cell lines were generated by the following protocol. 3×10⁵ Lenti-X 293T cells (Clonetech) were plated in 0.8 mL of media in a 12-well plate and incubated overnight at 37° C./5% CO₂. Packaging plasmid (0.4 μg, pMD), envelope plasmid (0.4 μg, pSP), and lentiviral transfer IRAK3-ePL plasmid (0.8 μg, IRAK3 sequence NM_007199.3) were mixed in 0.1 mL of Opti-MEM and incubated for 5 min. Simultaneously, 2.4 μL of Lipofectamine 2000 (Invitrogen) was added to 0.1 mL of Opti-MEM (Gibco) and incubated for 5 min. The plasmid DNA and lipofectamine were combined and the mixture was allowed to incubate for 20 min. DNA:Lipofectamine Opti-MEM mixtures were then added to previously plated cells dropwise and the cells were incubated for ~16 h at 37° C./5% CO₂. Following incubation, the media was removed and 1.2 mL of fresh media was added per well. Lenti-X 293T cells were incubated for ~30 h at 37° C./5% CO₂. 0.5×10⁶ 293T^{CRBN OE/GSPT1 G575N KI} cells were plated in 0.5 mL of media/well of a 12-well plate and incubated for ~16 h at 37° C./5% CO₂. Following incubation, media was removed from the Lenti-X 293T wells and passed through a 0.45 M filter. Part of the viral supernatant was used to transduce cells and the rest was stored at −80° C. Viruses were then added individually (0.5 mL virus) to each well of the 293T^{CRBN OE/GSPT1 G575N KI} cells, followed by addition of polybrene (10 mg/mL Millipore) to each well at a final concentration of 5.0 μg/mL. Cells were incubated for ~24 h at 37° C./5% CO₂. After aspirating media off plates, cells were washed with DPBS, trypsinized and plated in a 10 cm dish in 15 mL of media and 1 μg/mL puromycin. Following incubation of cells for ~72 h at 37° C./5% CO₂, media was aspirated off plates, and cells were washed with DPBS and trypsinized. The cells were plated in a 15 cm dish in 40 mL of media with 1.0 μg/mL Puromycin (Gibco) and incubated for ~72 h at 37° C./5% CO₂. Following incubation, media was removed and cells were washed with DPBS and trypsinized. The majority of cells were resuspended in Invitrogen freezing media and stored away (~6-8×10⁶ cells/vial).

IRAK3-ePL cellular dose response curve degradation assays were performed by the following protocol. Compounds to be tested were dispensed into a white 384-well tissue-culture treated plate using an acoustic liquid handler. Dilutions were prepared based on a 25 μL assay volume in duplicate 10 point 3-fold serial dilutions starting with a 10 μM dose. Negative control wells were included, which only contain 0.2% DMSO to calculate 100% signal. Positive control wells containing 30 μM Ataluren (luciferase inhibitor) were included to calculate the background signal level. All wells were backfilled to a final DMSO concentration of 0.2% to ensure DMSO uniformity across wells. IRAK3-ePL expressing cells (IRAK3-ePL Lenti-X 293T^{CRBN/GSPT1 G575N}) were washed, trypsinized, counted, and resuspended in fresh DMEM (Gibco) to give a cell concentration of 200,000 cells/mL. 25 μL of cells (5,000 cells/well) were dispensed into the wells of the 384-well plate prespotted with compounds in the previous step and incubated overnight at 37° C./5% CO₂. Following incubation, the 384-well plate was taken out of the incubator and left at room temperature for 30 min. InCELL hunter reagent was prepared according to manufacturer's instructions (EA reagent, lysis buffer, and substrate reagent in a 1:1:4 ratio, Cat #96-0002, DiscoverX), which was added to the 384-well plate 25 μL per well. Following the incubation of the plate for 1 h at room temperature, the luminescence signal was read using a ViewLux plate reader. Data was processed and analyzed in ActivityBase software. In short, the average luminescence values of the positive control wells were subtracted from the rest of the wells for background correction, and all luminescence values were normalized to the DMSO control wells. The average value of the DMSO control wells was set to equal 100% of the relative IRAK3-ePL protein levels. Normalized luminescence values were plotted on a graph as a function of compound concentration. Compound concentration was plotted on the x-axis and the corresponding relative IRAK3-ePL protein levels on the y-axis. The EC₅₀ value (the half-maximum effective concentration) of a compound for the degradation of the IRAK3-ePL was calculated using a four-parameter logistic model (sigmoidal dose-response model) (FIT=(A+{(B−A)/1+[(C/x)^{D}]})) where C is the inflection point (EC₅₀), D is the correlation coefficient, and A and B are the low and high limits of the fit, respectively).

The Dₘₐₓ was calculated by determining the maximum percentage loss of target protein following compound treatment.

### Example B2. IRAK Endogenous HTRF Degradation Assay

Cells (~50k) were plated in Cisbio 96-well low volume white plates (Cisbio: cat #66PL96005). Compounds were dissolved in DMSO and a 3-fold serial dilution was performed using a TECAN D300E. Cells were incubated with the compound overnight. Total-IRAK3 HTRF kit from Cisbio was used for degradation analysis (Cisbio: 63ADK101PEH). Cryptate and D2 antibodies were diluted in Detection buffer as per manufacturers recommendation. Then 2 μL of each solution was added to 16 μL lysate. Buffer control (lysis buffer detection buffer), Cryptate control (lysis buffer+cryptate antibody+detection buffer), and Negative control (lysis buffer+cryptate antibody+D2 antibody) were made as per manufacturers recommendation. Post incubation with antibodies the HTRF signal was measured using a Perkin Elmer Envision reader and the HTRF signal was calculated using formula: (Emission at 665 nm/Emission at 615 nm)*10,000. All HTRF values were normalized to the average value of DMSO. The average value of the DMSO control wells were set to equal 100% of the relative IRAK3 protein levels. Normalized luminescence values were plotted on a graph as a function of compound concentration. Compound concentration was plotted on the x-axis and the corresponding normalized IRAK3 protein levels on the y-axis. The EC₅₀ value (the half-maximum effective concentration) of a compound for the degradation of the IRAK3 was calculated using a four-parameter logistic model (sigmoidal dose-response model) (FIT=(A+{(B−A)/1+[(C/x)D]})) where C is the inflection point (EC₅₀), D is the correlation coefficient, and A and B are the low and high limits of the fit, respectively). The Yₘᵢₙ was calculated by determining the lowest percentage of target protein remaining following compound treatment. Dₘₐₓ was calculated from Yₘᵢₙ (% Dₘₐₓ=100−Yₘᵢₙ).

### Example B3. IRAK3 Biochemical Binding Assay

The LanthaScreen® Eu Kinase Binding assay was performed as described by the vendor (ThermoFisher Scientific Waltham, MA). Briefly, 100× solutions of compound were prepared in DMSO via serial dilution of the 10 mM stock solution in a 384-well reagent plate using 3-fold intervals to achieve final concentrations. 1 μL of the compound dilution series were added to the corresponding wells of a 384-well reagent plate containing 32.3 μL of 1× buffer (50 mM HEPES pH 7.4, 10 nM MgCl₂, 1 mM EGTA, 0.01% Brij-35). 5 μL of the buffer diluted compounds were transferred to the corresponding wells of a 384-well assay plate. 5 μL of 3× tracer was transferred to each well of the assay plate for a final tracer concentration of 10 nM. Finally, 5 μL of the 3× Eu-Anti-GST and IRAK3 mix was transferred to each well for a final concentration of 2 nM and 10 nM, respectively. Reactions were allowed to incubate for 1 hour at room temperature. TR-FRET signal of the interaction (λₑₓ40/λₑₘ 665/λₑₘ 615) was read at room temperature with a delay time of 100 μs and an integration time of 200 μs using an Envision plate reader. Background corrected emission signal ratios at each compound concentration were used to calculate percentage of inhibition (% o Inhibition). Plots of % Inhibition versus inhibitor concentrations were fit according to a dose-response equation (Eq. 1) to generate IC₅₀ and Hill slope values using Dotmatics software (Dotmatics, Bishops Storford, Hertfordshire, England).

$$\% Inhibition = ( \frac{100}{1 + {( \frac{{IC}_{50}}{[ I ]} )}^{n}} ) \qquad ( Eq . 1 )$$

Using these assays, IC₅₀, Dₘₐₓ, EC₅₀, and DC₅₀ values of the following compounds were determined. Dₘₐₓ is defined as the maximum percent degradation achieved and DC₅₀ is the concentration at which 50% degradation is achieved. Data is summarized in Table 2.

TABLE 2

| | | |
|---|---|---|
| IRAK3 | IRAK3 ePL | IRAK3 HTRF |
| binding | degradation | degradation |

| Compound | IC₅₀ | Dₘₐₓ | EC₅₀ | Dₘₐₓ | DC₅₀ |
|---|---|---|---|---|---|
| No. | (nM) | (%) | (μM) | (%) | (nM) |
| 1 | 111 | 8 | >10 | 41 | >30 |
| 2 | 139 | 25 | >10 | 35 | 0.64 |
| 3 | 186 | 13 | >10 | 42 | 3.99 |
| 4 | 120 | 26 | >10 | 21 | >30 |
| 5 | ND | 23 | >10 | 34 | >30 |
| 6 | 108 | 25 | >10 | 56 | 2.56 |
| 7 | 331 | 38 | >10 | 50 | >30 |
| 8 | 25 | 12 | >10 | 37 | >30 |
| 9 | 45 | 12 | >10 | 23 | >30 |
| 10 | 27 | 22 | >10 | 37 | >30 |
| 11 | 95 | 12 | >10 | 38 | >30 |
| 12 | 56 | 30 | >10 | 33 | >30 |
| 13 | 16 | 70 | 0.173 | 65 | 0.039 |
| 14 | 70 | 12 | >10 | 3 | >30 |
| 15 | 64 | 27 | >10 | 20 | >30 |
| 16 | 47 | 19 | >10 | 25 | >30 |
| 17 | 100 | 18 | >10 | 32 | >30 |
| 18 | 12 | 80 | 0.066 | 73 | 0.133 |
| 19 | 4 | 36 | >10 | 42 | 0.096 |
| 20 | 10 | 74 | 0.055 | 73 | 0.008 |
| 21 | 2 | 59 | 0.105 | 68 | 0.032 |
| 22 | 5 | 78 | 0.066 | 81 | 0.132 |

ND = not determined

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (IA): 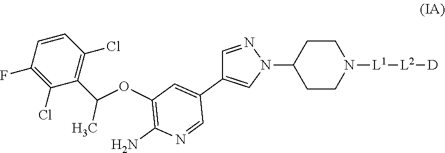
or a pharmaceutically acceptable salt thereof, wherein:
L¹ is —(CH₂CH₂O)ₙ— or a bond;
n is 1-10;
L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O;
D is 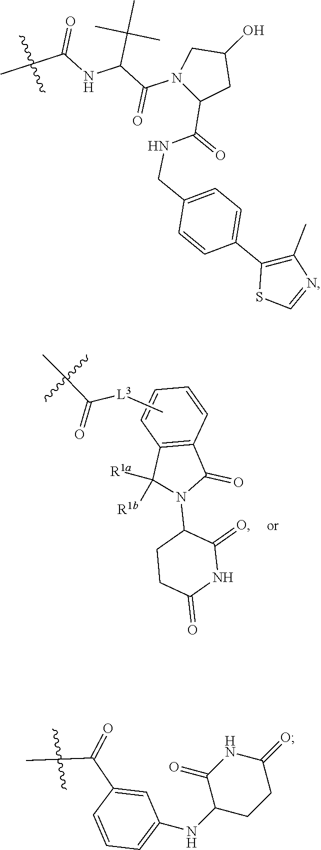
R^{1a} and R^{1b} are each H or are taken together to form an oxo; and
L³ is —CH₂O— or a bond.

2. The compound of claim 1, or a pharmaceutically acceptable salt thereof, wherein the compound is of Formula (I): 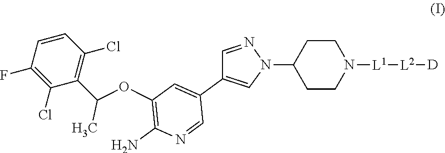
wherein:
L¹ is —(CH₂CH₂O)ₙ— or a bond;
n is 1-10;
L² is C₁-C₆ alkylene, —(C₁-C₆ alkylene)N(H)—, 6- to 12-membered spiro heterocyclylene, or a bond, wherein the heterocyclylene contains 1-3 heteroatoms selected from N and O;
D is 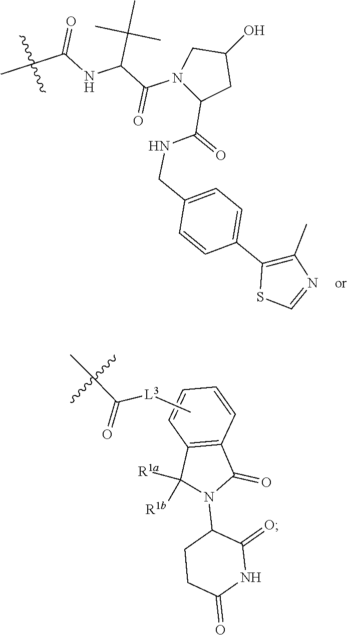
R^{1a} and R^{1b} are each H or are taken together to form an oxo; and
L³ is —CH₂O— or a bond.

3. The compound of claim 1 or 2, or a pharmaceutically acceptable salt thereof, wherein:
L¹ is —(CH₂CH₂O)ₙ—; and
n is 1-7.

4. The compound of claim 1 or 2, or a pharmaceutically acceptable salt thereof, wherein:
L¹ is a bond.

5. The compound of any one of claims 1-4, or a pharmaceutically acceptable salt thereof, wherein:
L² is C₁-C₃ alkylene, —(C₁-C₃ alkylene)N(H)—, 10- to 12-membered spiro heterocyclylene, or a bond, wherein the heterocyclylene contains 1-2 nitrogen atoms.

6. The compound of any one of claims 1-5, or a pharmaceutically acceptable salt thereof, wherein:
-L¹-L²- is

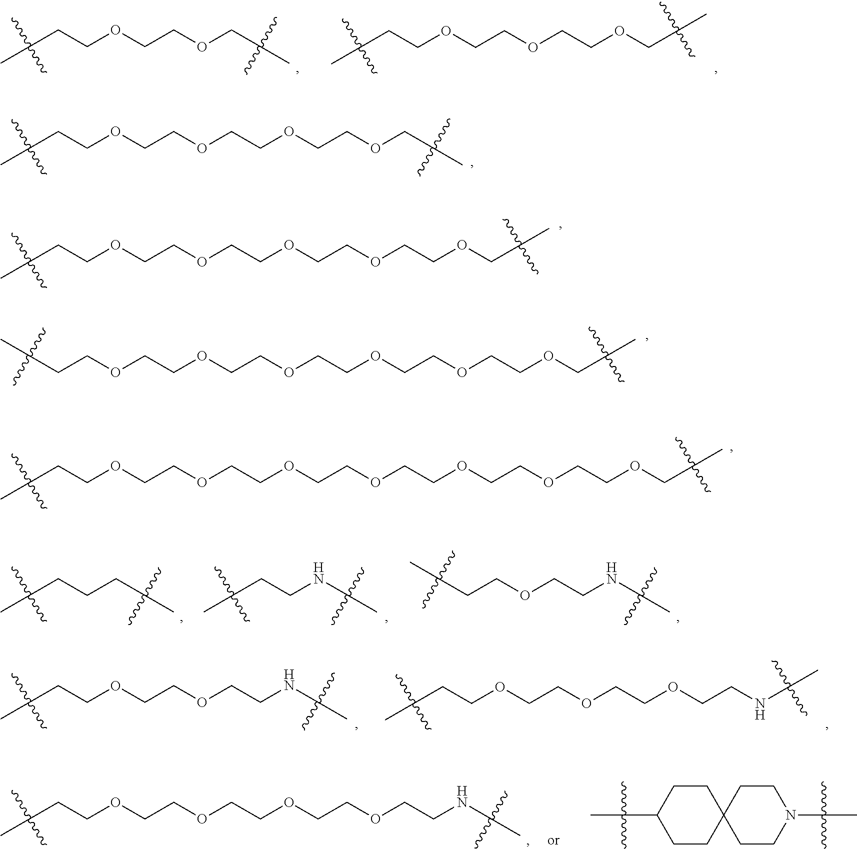

7. The compound of any one of claims 1-6, or a pharmaceutically acceptable salt thereof, wherein:
D is

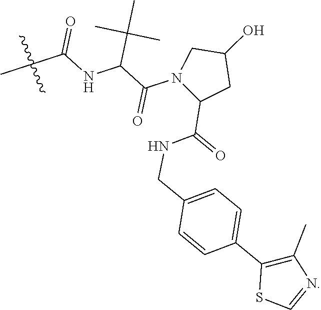 [SMILES: *C(=O)NC(C(=O)N1CC(O)CC1C(=O)NCc1ccc(-c2scnc2C)cc1)C(C)(C)C]

8. The compound of any one of claims 1-6, or a pharmaceutically acceptable salt thereof, wherein:
D is

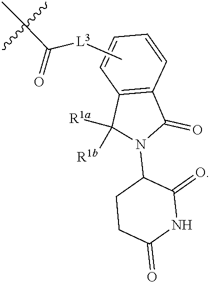

9. The compound of claim 8, or a pharmaceutically acceptable salt thereof, wherein:
R^{1a} and R^{1b} are each H.

10. The compound of claim 8, or a pharmaceutically acceptable salt thereof, wherein:
R^{1a} and R^{1b} are taken together to form an oxo.

11. The compound of any one of claims 8-10, or a pharmaceutically acceptable salt thereof, wherein:
L³ is —CH₂O—.

12. The compound of any one of claims 8-10, or a pharmaceutically acceptable salt thereof, wherein:
L³ is a bond.

13. The compound of any one of claims 8-12, or a pharmaceutically acceptable salt thereof, wherein: 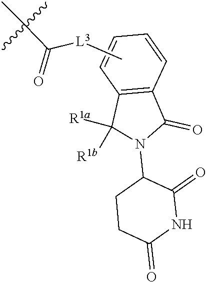
is

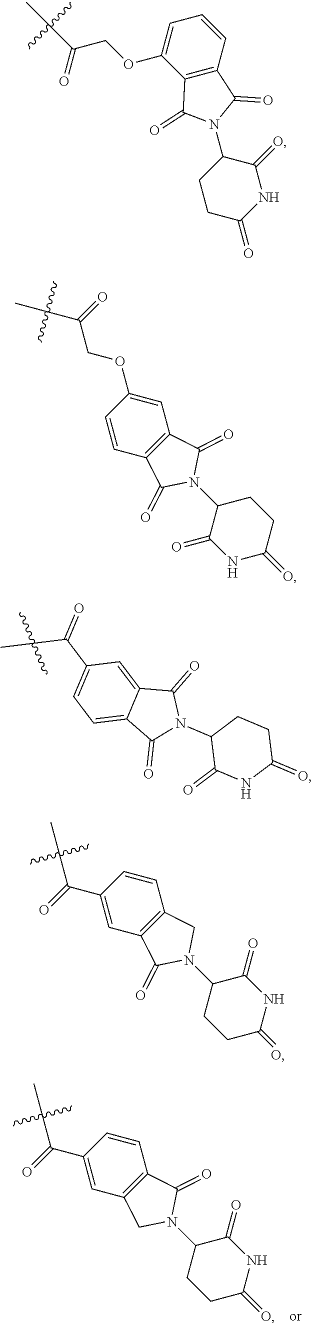 [SMILES: *C(=O)COc1cccc2c1C(=O)N(C1CCC(=O)NC1=O)C2=O ; *C(=O)COc1ccc2c(c1)C(=O)N(C1CCC(=O)NC1=O)C2=O ; *C(=O)c1ccc2c(c1)C(=O)N(C1CCC(=O)NC1=O)C2=O ; *C(=O)c1ccc2c(c1)C(=O)N(C1CCC(=O)NC1=O)C2 ; *C(=O)c1ccc2c(c1)CN(C1CCC(=O)NC1=O)C2=O]

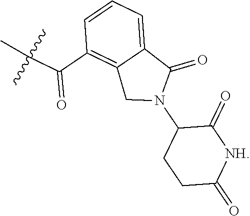 [SMILES: *C(=O)c1cccc2c1CN(C1CCC(=O)NC1=O)C2=O]

14. The compound of any one of claims 1-6, or a pharmaceutically acceptable salt thereof, wherein:
D is

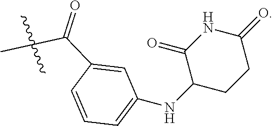 [SMILES: *C(=O)c1cccc(NC2CCC(=O)NC2=O)c1]

15. The compound of any one of claims 1-14, or a pharmaceutically acceptable salt thereof, wherein the compound is of Formula (II), (III), or (VII): 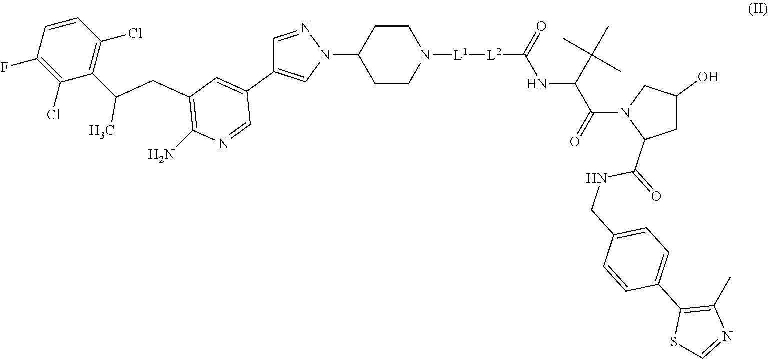
wherein:
L¹ is —(CH₂CH₂O)ₙ— or a bond;
n is 1-10; and
L² is C₁-C₆ alkylene; 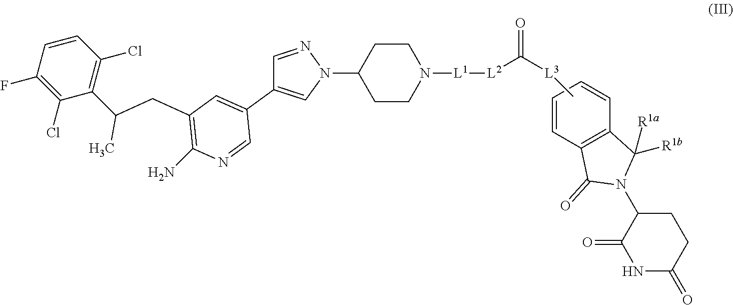
wherein:
L¹ —(CH₂CH₂O)ₙ— or a bond;
n is 1-10; and
L² is —(C₁-C₆ alkylene)-N(H)—, 6- to 12-membered spiro heterocyclylene, or a bond, wherein the heterocyclylene contains 1-3 heteroatoms selected from N and O; 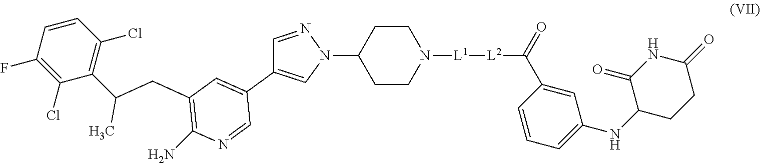
wherein:
L¹ is a bond; and
L² is 6- to 12-membered spiro heterocyclylene containing 1-3 heteroatoms selected from N and O.

16. A compound selected from the compounds of Table 1 and pharmaceutically acceptable salts thereof.

17. A pharmaceutical composition comprising the compound of any one of claims 1-16, or a pharmaceutically acceptable salt thereof, and a pharmaceutically acceptable excipient.

18. A method of modulating Interleukin-1 Receptor-Associated Kinase 3 (IRAK3) comprising contacting IRAK3 with an effective amount of the compound of any one of claims 1-16, or a pharmaceutically acceptable salt thereof, or the pharmaceutical composition of claim 17.

19. A method of treating cancer in a subject in need thereof, comprising administering to the subject an effective amount of the compound of any one of claims 1-16, or a pharmaceutically acceptable salt thereof, or the pharmaceutical composition of claim 17, optionally wherein the cancer is selected from bladder cancer, breast cancer, esophageal cancer, colon cancer, head and neck cancer, kidney cancer, lung cancer, pancreatic cancer, prostate cancer, melanoma, and gastric cancer.

20. A method of enhancing immunity in a subject receiving a vaccine, comprising administering to the subject an effective amount of the compound of any one of claims 1-16, or a pharmaceutically acceptable salt thereof, or the pharmaceutical composition of claim 17.
